(19) United States
(12) Patent Application Publication
(10) Pub. No.: US 2025/0019765 A1
(43) Pub. Date: Jan. 16, 2025

(54) RADIATION BIODOSIMETRY SYSTEMS

(71) Applicant: ARIZONA BOARD OF REGENTS ON BEHALF OF ARIZONA STATE UNIVERSITY, Scottsdale, AZ (US)

(72) Inventors: Joshua LaBaer, Chandler, AZ (US); Kristin Gillis, Mesa, AZ (US); Garrick Wallstrom, Mesa, AZ (US); Jin Park, Phoenix, AZ (US); Vel Murugan, Chandler, AZ (US); Mitch Magee, Chandler, AZ (US)

(21) Appl. No.: 18/896,122

(22) Filed: Sep. 25, 2024

Related U.S. Application Data
(63) Continuation of application No. 16/929,512, filed on Jul. 15, 2020.
Application No. 16/929,512 is a division of application No. 16/532,138, filed on Aug. 5, 2019, now Pat. No. 10,787,710.
Application No. 16/532,138 is a division of application No. 14/823,433, filed on Aug. 11, 2015, now Pat. No. 10,435,747.
(60) Provisional application No. 62/038,969, filed on Aug. 19, 2014.

(51) Int. Cl. C12Q 1/6883; C12Q 1/6827

(52) U.S. Cl. CPC C12Q 1/6883; C12Q 1/6827; C12Q 2600/156

(57) ABSTRACT

Disclosed herein are compositions and methods for accurately estimating the absorbed dose of radiation indicated by a subject based on the expression pattern of a panel of radiation-modulated (RM) genes at various time points following exposure of the subject to ionizing radiation.

### CROSS-REFERENCE TO RELATED APPLICATIONS

This application is a continuation of U.S. patent application Ser. No. 16/929,512, filed Jul. 15, 2020, which application is a divisional of U.S. patent application Ser. No. 16/532,138, filed Aug. 5, 2019 and now issued as U.S. Pat. No. 10,787,710, issued Sep. 29, 2020, which is a divisional of U.S. patent application Ser. No. 14/823,433, filed on Aug. 11, 2015 and now issued as U.S. Pat. No. 10,435,747, issued Oct. 8, 2019, and claims the benefit of U.S. Provisional Application No. 62/038,969, filed Aug. 19, 2014, the disclosures of which are hereby incorporated by reference in their entirety for all purposes.

### STATEMENT REGARDING FEDERALLY SPONSORED RESEARCH OR DEVELOPMENT

This invention was made with government support under HHSO100201000008C awarded by Biomedical Advanced Research and Development Authority. The government has certain rights in the invention.

### REFERENCE TO A SEQUENCE LISTING SUBMITTED VIA EFS-WEB

The contents of the electronic sequence listing (11262401477.xml; Size: 109,685 bytes; and Date of Creation: Sep. 24, 2024) is herein incorporated by reference in its entirety.

### BACKGROUND

Radiation exposure is one of the most serious hazards of the modern era. The health consequences to individuals and populations exposed to radiological incidents, accidental or otherwise, can range from negligible to fatal depending on the amount of radiation that is absorbed by an individual. Yet, it is often difficult or impossible to quickly determine the absorbed dose of radiation for an individual or population after a radiological event and thereby determine an appropriate course of treatment. This is particularly critical when large numbers of individuals are potentially affected by radiation exposure and must be quickly “triaged” to prioritize treatment strategies. Thus, there is a great need for systems that quickly estimate, post-hoc, the absorbed dose of radiation by an individual resulting from an ionizing radiation exposure incident.

### BRIEF SUMMARY OF THE INVENTION

Disclosed herein are compositions and methods for accurately estimating the absorbed dose of radiation suffered by a subject based on the expression pattern in RNA obtained from peripheral blood of a panel of radiation-modulated (RM) genes at various time points following exposure of the subject to ionizing radiation.

Accordingly in one aspect provided herein is a radiation biodosimetry assay system, comprising a plurality of nucleic acid amplification reactions comprising (i) mRNA or cDNA from a human subject suspected of (but not necessarily) suffering from radiation exposure; (ii) primer pairs capable of hybridizing under stringent conditions to mRNAs or cDNAs comprising the nucleotide sequences referred to in Table 4 or the complementary sequences thereof, wherein each primer pair hybridizes to a different one of the mRNAs or cDNAs; and (iii) a thermostable enzyme suitable for amplification of target amplicon sequences from the mRNAs or cDNAs. A mathematical algorithm that converts gene expression results to estimated absorbed dose of radiation.

In some embodiments the one or more nucleic acid amplification reactions further comprise detectably labeled TAQMAN® probes capable of hybridizing under stringent conditions to the mRNAs or cDNAs. In some embodiments the thermostable enzyme is a thermostable polymerase.

In some embodiments the mRNA is from a subject that was exposed to radiation about 4-hours to about seven days prior to the time at which a biological sample comprising the mRNA was obtained.

In another aspect provided herein is a radiation biomarker assay kit, comprising a nucleic acid probe set consisting essentially of nucleic acid probes that hybridize specifically with nucleic acid targets comprising at least one of the nucleotide sequences referred to in SEQ ID NOs: 1-29 or the complementary sequences thereof. In some embodiments the probe set comprises no more than 100 probes. In some embodiments the probe set consists of the nucleic acid probes that hybridize specifically with the nucleic acid targets.

In some embodiments the nucleic acid probe set comprises primer pairs and TAQMAN® probes suitable for qPCR analysis of mRNAs or cDNAs comprising at least one of the nucleotide sequences referred to in SEQ ID NOS: 1-29 or the complementary sequences thereof. In some embodiments the nucleic acid probes are provided in a multi-well plate. In some embodiments, where the nucleic acid probes are provided in a multi-well plate, at least two nucleic acid probes that hybridize to at least two different nucleic acid targets are in the same wells of the multi-well plate.

In some embodiments the kit also includes radiation exposure positive and negative control mRNA samples or cDNAs thereof. In another aspect provided herein is a method for assessing a dose of ionizing radiation absorbed by a subject, comprising (i) determining the mRNA expression levels of mRNAs comprising at least one of the nucleotide sequences referred to in SEQ ID NOs: 1-29 in a biological sample, comprising mRNA from the subject, to obtain an expression profile; and (ii) transforming the gene expression profile and when available, the duration of time from exposure to sample collection, into a measure of absorbed dose of radiation for the subject based on a mathematical algorithm. In one embodiment, the algorithm utilizes multiple random forest regression trees to estimate absorbed dose and confidence limits and then a top-level logic layer to combine outputs into a single estimated absorbed dose with confidence limits.

In some embodiments the method further includes treating the subject based on the estimated absorbed dose of radiation determined in step (ii).

In some embodiments the absorbed dose of ionizing radiation is determined within about seven days of subject exposure to ionizing radiation.

In some embodiments the method also includes a step of obtaining the biological sample from the subject prior to step (i).

In a further aspect provided herein is a method for radiation treatment triage of a subject in need thereof comprising (i) determining the mRNA expression levels of mRNAs comprising the nucleotide sequences referred to in at least one of SEQ ID NOs: 1-29 (or any other sequence identifier included herein, in any combination) in a biological sample comprising leukocyte mRNA from the subject to obtain a gene expression profile; and (ii) providing a suitable treatment for radiation exposure to the subject based on the expression levels of the genes.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### BRIEF DESCRIPTION OF THE DRAWINGS

The present invention will be better understood and features, aspects and advantages other than those set forth above will become apparent when consideration is given to the following detailed description thereof. Such detailed description makes reference to the following drawings, wherein:

FIG. 1A show tables listing a set of 28 radiation modulated (RM) genes and their expression pattern at various time points following absorption of ionizing radiation.

FIG. 1B show tables listing a set of 28 radiation modulated (RM) genes and their expression pattern at various time points following absorption of ionizing radiation.

FIG. 2 shows a time series (days 1-7) of plots of actual delivered ionizing radiation dosage values (0 Gy to 10 Gy) versus estimated absorbed dose of radiation based on changes in RM gene expression in peripheral blood

FIG. 3 shows a table providing the percentage accuracy (within 1 Gy) of the biodosimetry algorithm's absorbed radiation dose estimate based on expression of 29 RM genes in peripheral blood collected from rhesus macaque non-human primate (NHPs) at various time points (1-7 days) following exposure to irradiation doses ranging from 0 Gy to 10 Gy.

FIG. 4 shows a table providing the percentage accuracy (within 0.5 Gy) of the biodosimetry algorithm's absorbed radiation dose estimate based on expression of 29 RM genes in peripheral blood from NHPs at various time points (1-7 days) following exposure to irradiation doses ranging from 0 Gy to 10 Gy.

FIG. 5 shows scatter plots of changes in RM gene expression in male vs. female NHPs following radiation exposure of various doses and at different time points following radiation exposure. As shown, male and female RM gene expression responses were very closely correlated for the 29 RM genes.

FIG. 6 shows a table describing the sensitivity and specificity of the biodosimetry algorithm in various NHP and human irradiation models.

FIG. 7 shows an overview of an exemplary, non-limiting, embodiment of biodosimetry workflow illustrating the steps of: blood sample collection, RNA isolation, reverse transcription to obtain cDNA, pre-amplification of the cDNA, and qPCR assay of a RM biomarker and reference gene panel.

FIG. 8 shows a single regression tree in a random forest example. This tree generates an estimated absorbed dose using the relative expression levels of gene biomarkers, HBA2 and IL27RA, and the absolute expression level of gene biomarker, COCH.

FIG. 9 shows a schematic overview on human and NHP data sets, cross-species conversion approaches to utilize the NHP single-dose (SD) biodosimetry algorithm to predict absorbed dose in human in case of acute radiation exposure. Cross-species conversion algorithms were developed with human and NHP fractionated-dose (FD) models that showed a good cross-model compatibility with NHP SD models.

FIG. 10A shows a schematic overview on the approach to convert NHP fractionated dose (FD) data to the corresponding values in NHP single-dose (SD) data by 3-dimensional linear scaling of day, dose, and expression values.

FIG. 10B shows the optimal range (in blue) of dose/day scaling factors for 12 Gy/day-6 data points of individual biomarkers (top panels) and a unified scaling factor (bottom panel) for 29 tested biomarkers.

FIG. 11A shows dose prediction performances of a random forests model based on 7 correlated biomarker genes on NHP SD data (for day 3, as an example).

FIG. 11B shows dose prediction performances of a random forests model based on 7 correlated biomarker genes on converted NHP FD values across all days by matching day/cumulative dose, 3D scaling, and 3D scaling followed by multi-gene regression. Prediction accuracies within 1.0 Gy are shown.

FIG. 12A shows correlation of individual biomarker expression values between NHP fractionated dose (FD) and human TBI, magnitude of expressional changes across dose, and mean absolute difference (MAD) between NHP FD and human TBI data points.

FIG. 12B shows dose response curves of the top 4 inter-species correlated genes in NHP FD and human TBI data.

FIG. 13A shows dose prediction performances of a random forests model based on 10 inter-species biomarker genes on NHP FD data.

FIG. 13B shows dose prediction performances of a random forests model based on 10 inter-species biomarker genes on unconverted and converted human TBI values by value shift, and value shift followed by multi-gene regression. Prediction accuracies within 1.0 Gy are shown.

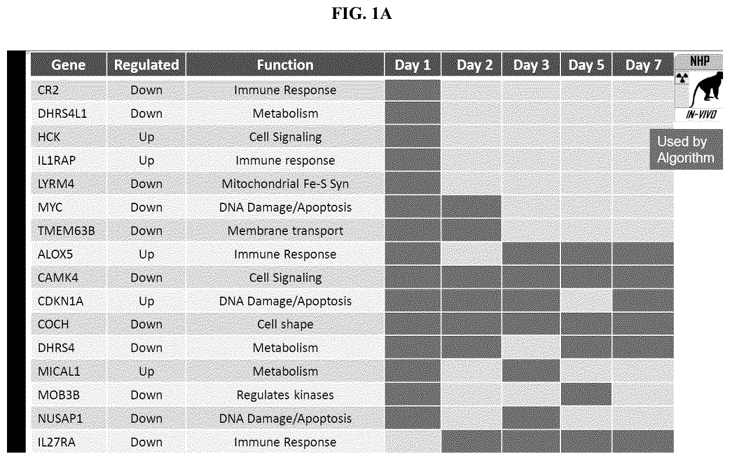

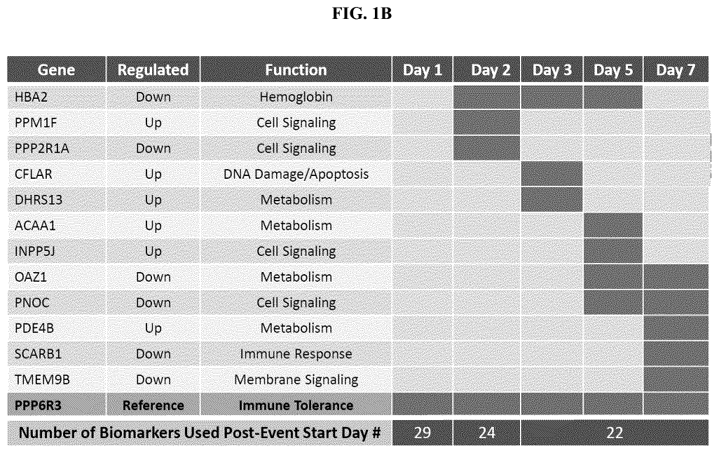

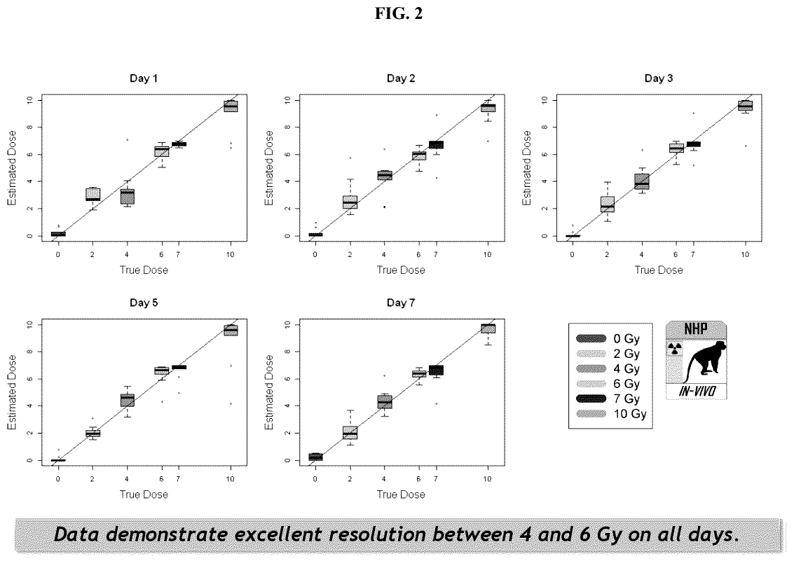

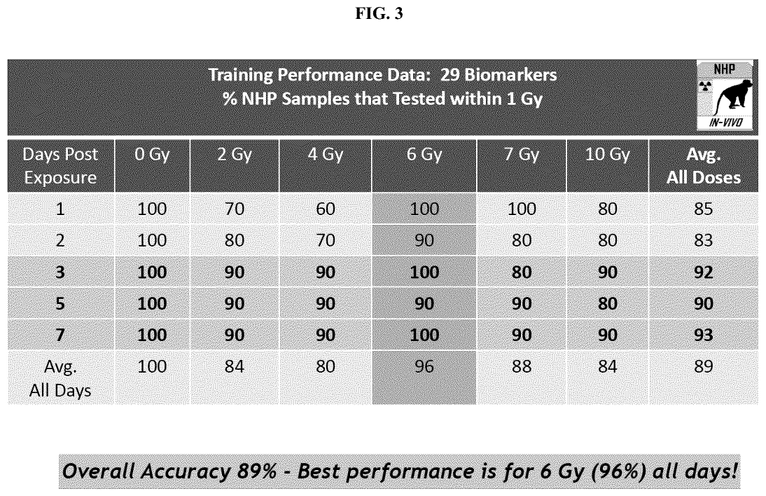

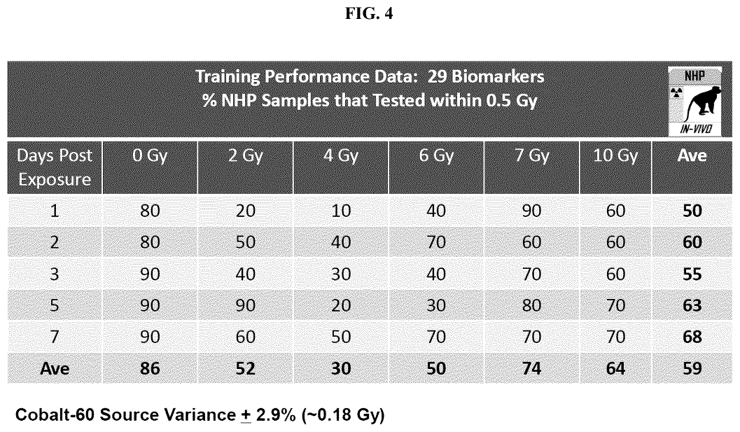

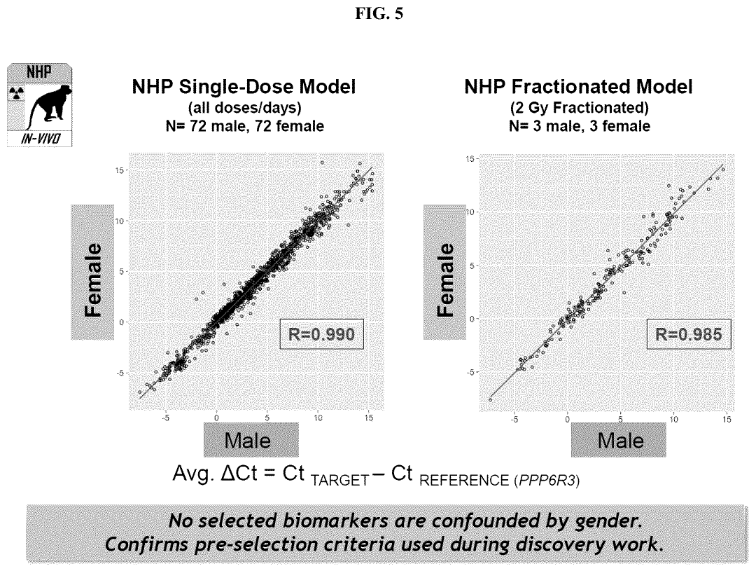

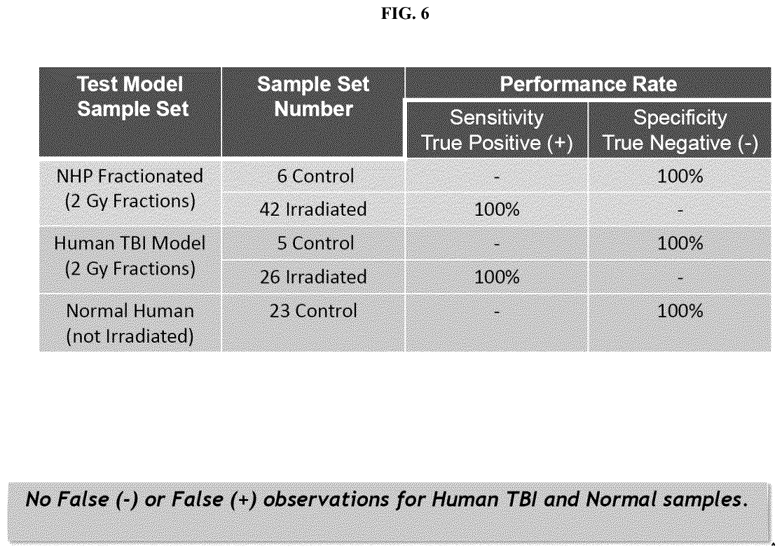

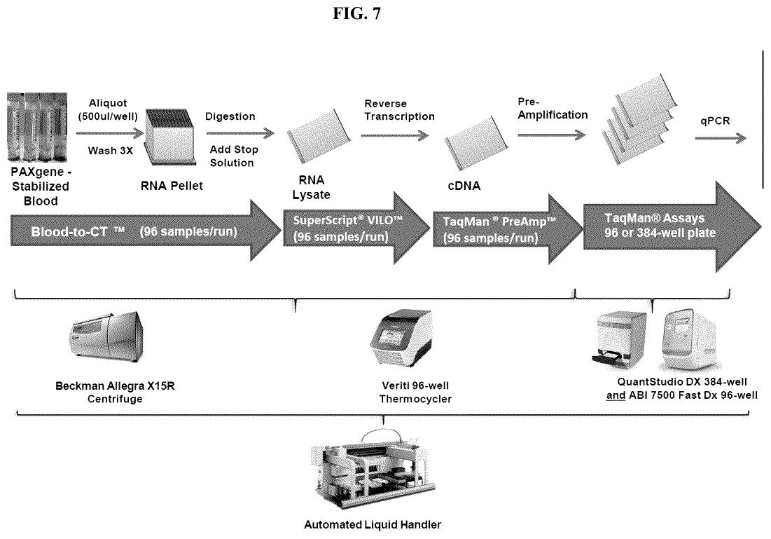

### DETAILED DESCRIPTION

In General. Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The Invention. The present invention provides methods for estimating absorbed dose of ionizing radiation by a subject, which includes the steps of: (i) determining the mRNA expression levels of mRNAs comprising the nucleotide sequences referred to in SEQ ID NOs: 1-29 in a biological sample comprising peripheral blood mRNA collected from the subject to obtain an expression profile; and (ii) transforming the gene expression profile and when available, the duration of time from exposure to sample collection into an estimated absorbed dose of ionizing radiation and confidence limits for a subject based on a mathematical algorithm. For each of several durations for which training data were available, one primary random forest was developed to estimate absorbed dose of radiation. Additional secondary random forests were developed to provide more accurate dosimetry in narrow dosage intervals. The top-level logic layer uses the primary random forest to generate an initial estimate of absorbed dose of radiation, and based on that value, may select additional random forests to construct more refined estimates of absorbed dose, with confidence limits.

In some embodiments the method also includes treating the subject based on the absorbed dose of ionizing radiation determined in step (ii) above. In some embodiments the absorbed dose of ionizing radiation is determined within about seven days of exposure to the ionizing radiation, e.g., within about 30 minutes, 1 hour, 3 hours, 6 hours, 8 hours, 24 hours, 1 day, 2 days, 3 days, 4 days, 5 days, 6 days, or another time period within seven days following ionizing irradiation. In some embodiments, the absorbed dose of ionizing radiation is within the range of about 0.5 Grays (Gy) to about 10 Gy, e.g., about 1 Gy, 2 Gy, 3 Gy, 4 Gy, 6 Gy, 7 Gy, 8 Gy, 9 Gy, or another absorbed dose of ionizing radiation from about 0.5 Gy to about 10 Gy.

In some embodiments, the method also includes obtaining the biological sample from the irradiated subject prior to step (i) above.

In one embodiment, a whole blood sample, or other blood fraction containing lymphocytes, (including a finger stick or POC device) is collected from a subject known to be or suspected of being irradiated into a PAXGENE™ Blood RNA tube. The PAXGENE™ Blood RNA contains an additive that stabilizes in vitro gene expression and RNA degradation. Subsequently, RNA is extracted from the stabilized blood sample by using a Stabilized BLOOD-TO-CT™ Nucleic Acid Preparation Kit for qPCR (Life Technologies, Inc.). The RNA sample is then subjected to reverse transcription, e.g., using the INVITROGEN™ SUPERSCRIPT® VILO™ (Variable Input, Linear Output) cDNA synthesis kit (Life Technologies, Inc.) or equivalent kit. Afterwards, the resulting cDNA is pre-amplified using a TAQMAN® probe PreAmp Master Mix Kit (Life Technologies, Inc.) and the pre-amplified cDNA is then assayed by TAQMAN® probe-based qPCR in a 96-well or 384-well format using QUANTSTUDIO™ Dx or ABI7500 Fast Dx quantitative Real-Time PCR Instruments (Life Technologies, Inc.). Typically, expression levels of RM mRNAs will be expressed as a difference in Cr between a test gene and a reference (“housekeeping”) gene C_{T}.

In some embodiments the panel of RM mRNAs to be assayed include at least some combination of mRNAs for one or all of the following (human) genes: CR2 (SEQ ID NO: 1), DHRS4L1 (SEQ ID NO: 2), HCK (SEQ ID NO: 3), IL2RAP (SEQ ID NO: 4), LYRM4 (SEQ ID NO: 5), MYC (SEQ ID NO: 6), TMEM63B (SEQ ID NO: 7), ALOX5 (SEQ ID NO: 8), CAMK4 (SEQ ID NO: 9), CDKN1A (SEQ ID NO: 10), COCH (SEQ ID NO: 11), DHRS4 (SEQ ID NO: 12), MICAL2 (SEQ ID NO: 13), MOB3B (SEQ ID NO: 14), NUSAP1 (SEQ ID NO: 15), IL27RA (SEQ ID NO: 16), HBA2 (SEQ ID NO: 17), PPM1F (SEQ ID NO: 18), PPP2R1A (SEQ ID NO: 19), CFLAR (SEQ ID NO: 20), DHRS13 (SEQ ID NO: 21), ACAA1 (SEQ ID NO: 22), INPP5J (SEQ ID NO: 23), OAZ1 (SEQ ID NO: 24), PNOC (SEQ ID NO: 25), PDE4B (SEQ ID NO: 26), SCARB1 (SEQ ID NO: 27), TMEM9B (SEQ ID NO: 28), PPP6R3 (SEQ ID NO: 29), CD97, TEX10, SPECC1, ALAS2, ALPK1, ESD, GPR183, ESD, PPM1K, and SLC6A6.

In other embodiments RM mRNAs to be assayed can include at least some combination of one or all of the following genes: ADAM17, AKT1, ANK1, ANXA3, ARHGAP26, ARID4A, ATG2A, ATIC, BCL11A, BCL6, BID, CFLAR, CIT, CPVL, CYTH4, DDB2, DDX58, DTL, EHBPL1, FCGR2A, FGR, HPRT1, HSP90AB1, HTRA2, IDOL, IRF1, JMJD1C, KIAA0101, LARP4B, LRRC6, LYN, MAP3K11, MAPK3, MDM1, MKNK1, MXD1, NAIP, NFE2L2, NRG1, NUSAP, PCNA, PGK1, PMP22, RARA, RNASE6, RPL13A, RPL6, RPS14, SP110, SPOCK2, TAPBP, TBP, TCF3, TNFRSF1A, TNFRSF1B, TNFSF14, USP38, WDR48, XAF1, ZAK, NPM1, CPSF1, COASY, DNAJC10, DYNLRB1, ELK4, GPRIN, NDE1, PGS1, PPM1K, and PTAFR. In some embodiments, the reference gene to be assayed is PPP6R3. In other embodiments the reference gene to be assayed may be USP38, WDR48 or LARP4B or some combination thereof.

In some embodiments, qPCR reactions are multiplexed such that multiple mRNAs (including a reference mRNA) are assayed in a single qPCR reaction.

Also disclosed herein is a method for radiation treatment triage of a subject in need thereof, which includes the steps of: (i) determining the mRNA expression levels of mRNAs comprising the nucleotide sequences referred to in any of SEQ ID NOS: 1-29 (or any combination of any other SEQ ID NO provided herein) in a biological sample comprising mRNA from the subject to obtain an expression profile; and (ii) providing a suitable treatment for radiation exposure to the subject based on the expression levels of the genes. Exemplary treatments for radiation exposure based on radiation dosage are shown in Table 1 below:

TABLE 1
Exemplary treatments for radiation exposure based on radiation dosage.

| | | | | | Very | |
|---|---|---|---|---|---|---|
| Symptoms and | | Mild | Moderate | Severe | severe | Lethal (a) |
| treatment strategy | | (1-2 Gy) | (2-4 Gy) | (4-6 Gy) | (6-8 Gy) | (>8 Gy) |
| Vomiting | Onset | After 2 hr. | After 1-2 | Within | Within 30 | Within 10 min. |
| | Incidence | 10-50% | hrs. | 1 hr. | min. | 100% |
| | | | 70-90% | 100% | 100% | |
| Diarrhea | Onset | None | None | Mild | Heavy | Heavy |
| | Incidence | | | 3-8 hrs. | 1-3 hrs. | Within min. −1 hr. |
| | | | | <10% | >10% | almost 100% |
| Headache | Onset | Slight | Mild | Moderate | Severe | Severe |
| | Incidence | | | 4-24 hrs. | 3-4 hrs. | 1-2 hrs. |
| | | | | 50% | 80% | 80-90% |
| Conscious- | Onset | Alert | Alert | Alert | Possibility | Unconsciousness |
| ness | Incidence | | | | of | by order of seconds |
| | | | | | impairment | or minutes |
| | | | | | | Seconds-minutes |
| | | | | | | 100% (>50 Gy) |
| Body | Onset | Normal | Increased | Fever | High fever | High fever |
| Temperature | Incidence | | 1-3 hrs. | 1-2 hrs. | <1 hrs. | <1 hrs. |
| | | | 10-80% | 80-100% | 100% | 100% |
| Treatment | | Outpatient | Observation | Treatment | Treatment | Palliative treatment |
| Strategy | | observation | at general | at | at | (a) (advanced |
| | | | hospital, | specialized | specialized | medical care |
| | | | treatment at | Hospital | hospital | including stem cell |
| | | | specialized | | | transplantation) |
| | | | hospital if | | | |
| | | | required | | | |

Also described herein is a radiation biodosimetry assay system that includes multiple nucleic acid amplification reactions containing the following: (i) mRNA or cDNA from a human subject suspected of suffering from radiation exposure; (ii) primer pairs capable of hybridizing under stringent conditions to mRNAs or cDNAs comprising the nucleotide sequences referred to in SEQ ID NOS: 1-29 (or any other SEQ ID NO provided herein), or the complementary sequences thereof, wherein each primer pair hybridizes to a different one of the mRNAs or cDNAs; and (iii) A mathematical algorithm the converts gene expression results to estimated absorbed dose of radiation.

In one embodiment, the mathematical algorithm of the present invention The Radiation Biodosimetry Absorbed Dose Estimation algorithm described herein takes as input sample qPCR data, sample barcode, and available information about the date and time of the exposure event and sample collection. The primary output of the algorithm is an absorbed dose report that contains an estimated absorbed dose and a dose interval that provides a range of dose values for the patient based on prediction intervals. The algorithm contains 6 basic steps, which are summarized in Table 2.

In Step 1, patient qPCR data are combined with the available information about the date and time of the event and sample collection using the patient barcode.

In Step 2, several quality control metrics are calculated for the qPCR data. Depending on the values of these metrics, the algorithm may determine that a sample requires re-testing. If the sample does not require re-testing, the quality control metrics will be utilized in the estimation of absorbed radiation dose, and in particular may affect the estimation interval.

In step 3, the qPCR data are checked against expected ranges for each biomarker.

In step 4, quality control metrics and the results of the biomarker range checks are used to determine whether specific biomarker values are invalid and whether sufficient biomarker values are valid for dose estimation.

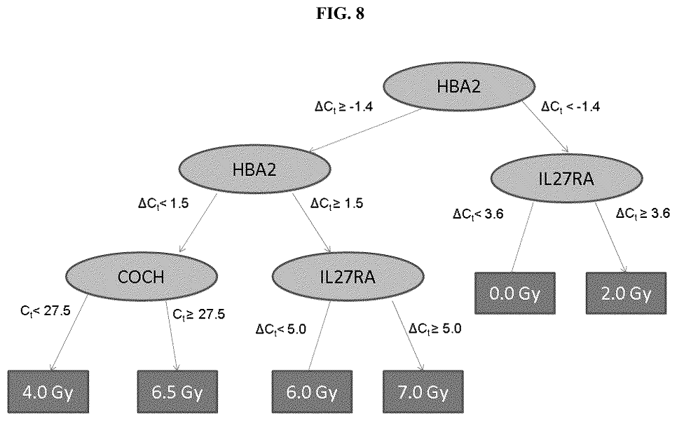

In step 5, the qPCR data and the available temporal information for the event and sample collection are processed through a random forest-based mathematical algorithm that yields an estimated dose and a dose interval. The mathematical algorithm in Step 5 utilizes Random forests™ method, introduced by Breiman (2001). Random forests is a popular machine-learning tool for prediction that combines large numbers of classification or regression trees to yield accurate and robust predictions. A random forest is a collection of classification or regression trees that we denote by R={T₁, . . . , Tₙ}. The input to the forest is a vector X of relative and/or absolute expression levels of a set of genes. In a regression random forest, each tree Tᵢ takes X as input and outputs an estimate of absorbed dose, Tᵢ(X). For example, FIG. 8 depicts a single tree in a random forest that utilizes the relative expression levels for two gene biomarkers (HBA2 and IL27RA) and the absolute expression level for one gene biomarker (COCH). The estimate of absorbed dose from a regression random forest R is then the average of estimated absorbed doses from the trees within the forest. We write this estimate as:

$$A D = R ( X ) = \frac{1}{n} \times {\sum }_{i = 1}^{n} {T}_{i} ( X )$$

In a classification random forest, samples are partitioned into several non-intersecting groups. For example, samples may be partitioned based upon dose and each group then represents an interval for the absorbed dose. Each tree Tᵢ takes as input X and outputs the identity of a single group, Tᵢ(X). The output from the classification random forest is a probability distribution on the set of groups, where the probability assigned to each group is the proportion of trees that yield the group.

The inputs to the mathematical algorithm in Step 5 are a set of absolute and relative expression levels, X for a set of genes, and a probability distribution π that reflects the uncertainty in D, the duration of time from irradiation to sample collection. If the duration of time is known precisely, π will be a point mass distribution on that known duration of time. If the duration is only known to fall within an interval, then π may be any probability distribution on that interval such as a uniform distribution or a symmetric triangular distribution. If the duration is entirely unknown or not provided, then π may be calculated using a classification random forest that takes as input X and yields as output a probability distribution over a fixed set of duration values.

The outputs are an estimated absorbed dose, AD and a 95% prediction interval for the absorbed dose, (AD_{low}, AD_{high}). For NHP samples that were irradiated with a single acute dose (NHP SD), these outputs are computed in two steps. First, we compute an initial estimate of absorbed dose, AD^{I}. Secondly, we correct for bias in the estimate to yield the final estimate of absorbed dose, AD and generate the 95% prediction interval.

A novel aspect of our algorithm is the use of multiple random forests for each of several fixed durations, D₁, . . . , Dₖ. For duration Dᵢ, we use nᵢ random forests, denoted by RF_{i,1}, . . . , RF_{i,n}_{ᵢ}, to construct initial estimates of absorbed dose. A decision tree Tᵢ combines the outputs from RF_{i,1}, . . . , RF_{i,n}_{ᵢ} into a single initial estimate of absorbed dose. One additional random forest, denoted by RFᵢ^{E}, is a quantile regression random forest for error that uses the expression values X′ and the output from Tᵢ for bias correction and construction of prediction intervals. These nᵢ+1 random forests utilize different, but possibly overlapping sets of genes, may be trained on different sets of samples and may include both regression forests and classification forests. Hence, if the duration is known to equal Dᵢ, the initial estimate of absorbed dose, denoted by AD^{I}(Dᵢ), is computed as:

$$A {D}^{I} ( {D}_{i} ) = {T}_{i} ( R {F}_{i , 1} ( {X}^{′} ) , … , {RF}_{i , {n}_{i}} ( {X}^{′} ) )$$

This estimate and the transformed expression levels X′ are then passed to the random forest RFᵢ^{E}. The output from RFᵢ^{E} is the conditional probability distribution for the error in the estimate AD^{I}(Dᵢ). We denote the cumulative distribution function for this conditional distribution by F(·|D=Dᵢ). The bias corrected estimate of absorbed dose is then AD(Dᵢ)=AD^{I}(Dᵢ)−F⁻¹(0.5). If the duration is known to equal D* where Dᵢ<D*<Dᵢ₊₁, the estimated absorbed dose is computed as:

$$A D ( {D}^{*} ) = \frac{{D}_{i + 1} - {D}^{*}}{{D}_{i + 1} - {D}_{i}} \times A D ( {D}_{i} ) + \frac{{D}^{*} - {D}_{i}}{{D}_{i + 1} - {D}_{i}} \times A D ( {D}_{i + 1} )$$

The final estimate of absorbed dose is computed by averaging over the probability distribution π, that is, AD=∫AD(D)×π(D)dD.

Similarly, we define F(·|D=D*) by:

$$F ( \cdot \vert D = {D}^{*} ) = \frac{{D}_{i + 1} - {D}^{*}}{{D}_{i + 1} - {D}_{i}} \times F ( \cdot \vert D = {D}_{i} ) + \frac{{D}^{*} - {D}_{i}}{{D}_{i + 1} - {D}_{i}} \times F ( \cdot \vert D = {D}_{i + 1} )$$

The 95% prediction interval for the absorbed dose is then found by solving the equations:

$$\int F ( {e}_{1} \vert D ) \times π ( D ) d D = 0.025 , and$$

$$\int F ( {e}_{2} \vert D ) \times π ( D ) d D = 0.975$$

for e₁ and e₂, respectively, and setting AD_{low}=AD−e₂ and AD_{high}=AD−e₁.

In step 6, an absorbed dose estimation report is constructed utilizing the estimated dose and dose interval.

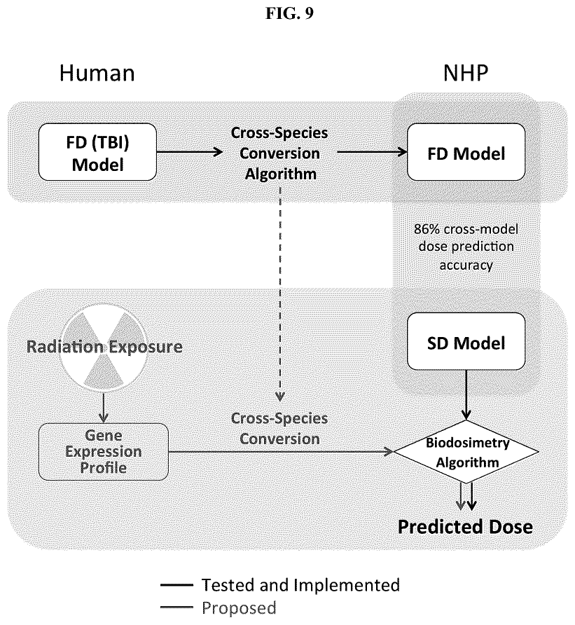

In actual events of acute radiation exposure, to predict absorbed dose of human samples with the NHP-based biodosimetry algorithm, conceptually, gene expression measurements of each biomarker in a human sample need to be transformed by cross-species (i.e. human to NHP) algorithms. Ideal cross-conversion models could be built on two directly comparable single-dose (SD) data sets in human and NHP. However, due to practical difficulties in obtaining human blood samples with single acute irradiation, as an alternative, we obtain samples from human subjects who undergo total body irradiation (TBI). Unlike the acute single-dose (SD) irradiation that we used for development of a biodosimetry algorithm, these subjects under a fractionated dose (FD) schedule were irradiated three times (1.2 Gy each) a day for 6 days. Therefore, we obtained gene expression data from NHP-equivalents of human TBI subjects that underwent the identical fractionated irradiation, and developed novel gene-specific cross-species conversion algorithms. These algorithms will be used to transform human values prior to dose prediction (FIG. 9).

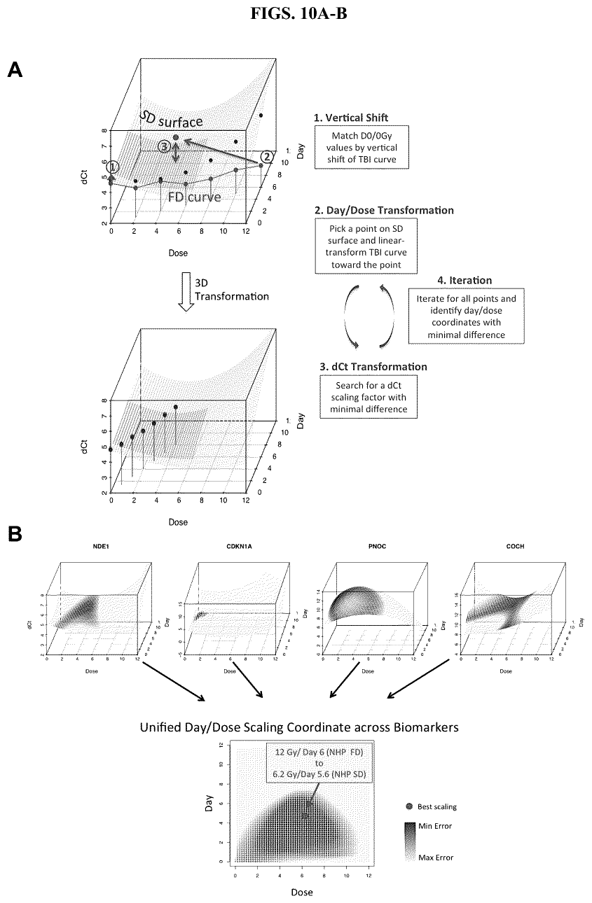

Unlike SD models that measure gene expression levels over the time after a single acute irradiation, data from a FD model has a linear relationship between cumulative dose and day. Therefore, prior to development of cross-species conversion algorithms based on human and NHP FD data sets, we first examined whether expression profiles of biomarker genes in NHP FD model were comparable to those in NHP SD model and thus could predict absorbed dose via the NHP SD model-based biodosimetry algorithm. For meta analyses of FD and SD data sets, we developed a three-dimensional (3D) curve fitting strategy to match the FD data to the SD data. Specifically, for each biomarker b we generate an FD curve of the mean expression level of NHP FD samples (2 Gy per day for 6 days) as a function of cumulative dose and day, FD_{b}(dose, day), and a SD response surface of the mean expression level of NHP SD samples (0 to 6 days, 0 to 7 Gy) as a function of dose and day, SD_{b}(dose, day) (FIG. 10A). First, the entire FD curve for each biomarker is shifted to match the mean basal level (i.e. 0 Gy/Day 0) of SD values, which produces a scaling factor α_{b} for expression values for each biomarker. The shifted FD curve is denoted FD_{b}′(dose, day)=FD_{b}(dose, day)+α_{b}, where α_{b}=SD_{b}(0, 0)−FD_{b}(0, 0). Second, for each biomarker, optimal biomarker-specific dose and day scaling factors, β_{b,dose} and β_{b,day}, are found that minimize the sum of absolute differences between the SD and scaled FD data.

Specifically, β_{b,dose} and β_{b,day} minimize the following expression,

$${\sum }_{d 1 , d 2} \vert \frac{F {D}_{b}^{′} ( d1 , d 2 )}{F {D}_{b}^{′} ( 12 , 6 )} \times S {D}_{b} ( {β}_{b , d o s e} \times d 1 , {β}_{b , d a y} \times d 2 ) - S {D}_{b} ( {β}_{b , d o s e} \times d 1 , {β}_{b , d a y} \times d 2 ) \vert$$

By repeating this for all biomarkers and searching for common dose and day scaling factors that minimize the sum of absolute differences across biomarkers, doses and days, unified FD to SD scaling factors of β_{dose}=0.517 for dose (i.e. 12 Gy to 6.2 Gy) and β_{day}=0.933 for day (i.e. day 6 to day 5.6) (FIG. 10B) are obtained.

Mathematically, β_{dose} and β_{day} are defined to minimize

$${\sum }_{b} {\sum }_{d 1 , d 2} \vert \frac{F {D}_{b}^{′} ( d 1 , d 2 )}{F {D}_{b}^{′} ( 12 , 6 )} \times S {D}_{b} ( {β}_{d o s e} \times d 1 , {β}_{d a y} \times d 2 ) - {SD}_{b} ( {β}_{d o s e} \times d 1 , {β}_{d a y} \times d 2 ) \vert .$$

Third, linear transformation of FD curves by the scaling factors yields the converted expression values,

$$F {D}_{b}^{″} ( d 1 , d 2 ) = \frac{F {D}_{b}^{′} ( d 1 , d 2 )}{F {D}_{b}^{′} ( 12 , 6 )} \times S {D}_{b} ( {β}_{d o s e} \times d 1 , {β}_{d a y} \times d 2 )$$

### (FIG. 10A).

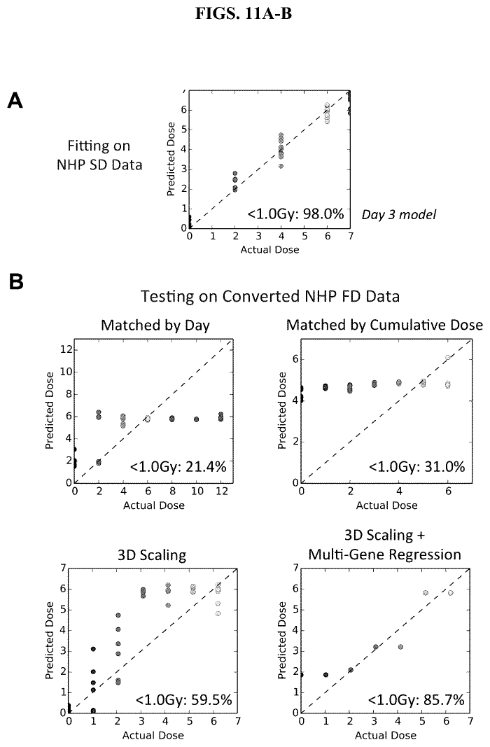

To test the conversion strategy from NHP FD to NHP SD data, based on seven biomarkers (COCH, DHRS4L1, IL27RA, INPP5J, PNOC, SCARB1, and TEX10 in this example) with correlated dose responses between the data sets, random forests dose prediction models were generated on NHP SD data, which showed 84% to 98% dose prediction accuracy across days for the model fitting on NHP SD data (FIG. 11A). When expression values of NHP FD data were converted by matching days or doses and then applied to the NHP SD random forests model, dose prediction accuracies within 1.0 Gy were only 21% and 31%, respectively. After the FD values were transformed by the 3D scaling factors (i.e. for expression value, dose, and day), the accuracy was increased to 60% (FIG. 11B).

To increase performance of conversion algorithm, we explored a multi-gene regression approach that utilizes linear combinations of gene expression values rather than the expression values of individual biomarkers. This concept has been applied to predict missing values in large gene expression data sets. Since the biomarkers are functionally related within the key biological pathways related to radiation response, we hypothesized that expression profiles of other biomarkers could be informative in predicting expression values of a given gene. We employed Ridge regressions that provide robustness by constraining the size of coefficients by minimizing the summed squares of residuals and coefficients. By using converted NHP FD values by 3D scaling, a multi-gene regression model for each biomarker was generated with all seven genes that were used to build NHP SD biodosimetry algorithm. When the predicted values by the multi-gene regression models were applied to NHP SD algorithm, dose prediction accuracy was increased substantially to 86% (FIG. 11B).

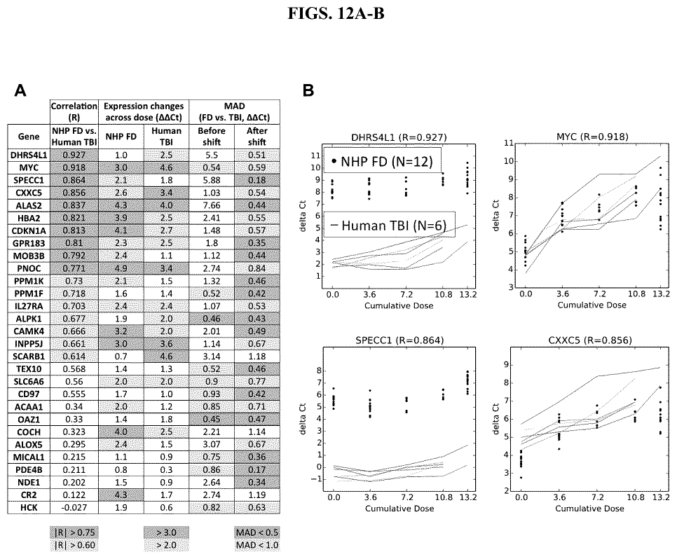

Absolute gene expression values of biomarker genes are highly variable between NHP and human, and, thus, using an NHP biodosimetry algorithm to predict absorbed dose in human requires another step of cross-species expression value transformation from human to NHP. To explore the conversion strategies, we compared two data sets, for human and NHP, that were obtained from subjects treated with an identical irradiation schedule (3 times of irradiation at 1.2 Gy per day for 4 days), which is being used for total body irradiation (TBI) in clinical therapeutic setting. Among 29 biomarkers tested, although 17 genes had inter-species correlation coefficient above 0.6 between these two data sets (FIG. 12A), many genes showed substantial differences in absolute expression levels across doses (FIG. 12B). Therefore, we calculated the mean difference of expression for each biomarker and then applied the value to shift the entire expression values the gene across doses. This process decreased the mean absolute differences to less than 1.0 ΔCt for the majority of 29 biomarkers (FIG. 12A, last two columns).

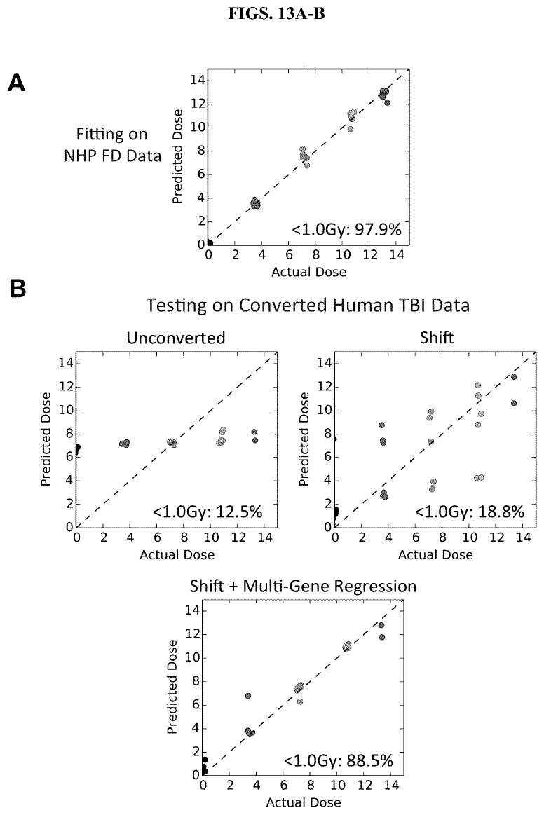

To test the conversion strategy from human TBI to NHP FD data, based on 10 biomarkers (DHRS4L1, MYC, SPECC1, CXXC5, ALAS2, HBA2, CDKN1A, GPR183, MOB3B, and PNOC in this example) with inter-species correlation above 0.75 (FIG. 12A), random forests dose prediction models were generated on NHP FD data, which showed a 98% dose prediction accuracy during the model fitting on NHP FD data (FIG. 13A). Appling the converted human TBI values to NHP FD random forests model, prediction accuracy within 1.0 Gy was 13%. When expression values of human TBI data were vertically shifted by the predetermined shift factors and then applied to the NHP FD random forests model, dose prediction accuracies within 1.0 Gy was increase marginally to 19%.

We then tested whether the multi-gene regression approaches could improve the cross-species conversion process. As previously done for NHP FD to NHP SD conversion, we also employed Ridge regressions. By using converted human TBI values by vertical shifting factors, a multi-gene regression model for each biomarker was generated with all 10 genes that were used to build the NHP FD biodosimetry algorithm in this example. When the predicted values by the multi-gene regression models were applied to NHP FD algorithm, dose prediction accuracy was increased substantially to 89%.

TABLE 2
Algorithm Function.

| | | | | | | |
|---|---|---|---|---|---|---|
| Step 1 | Combine | Traceability | Barcode | Unique barcode | Adds all needed | Flags data for operator |
| | Patient | | | | information to | intervention |
| | Barcode with | | | | qPCR data | |
| | Date/Time | | | | | |
| | Event | | | | | |
| Step 2 | Quality Control | Negative | 96-Well Prep Plate No | Ct (Ref)(2) > 37 | Cross- | Retest Samples |
| | Check | Control | Template Control | | Contamination in | |
| | | | (NTC) or Reagent | | Sample Prep | |
| | | | Blank | | | |
| | | Positive | qPCR Standard | 1. Ct (Ref) ± 1 | 1. LLOD Verified | Flags data - may affect |
| | | Controls | Curve: 10, 0.1, 0.01, | each conc. | 2. Reproducibility | Dose Estimate and |
| | | | 0.001 ng/μL | 2. Amplification | Verified | Dose interval |
| | | | (Based on standard | Efficiency | 3. Amplification | Or |
| | | | pooled RNA). | | verifies | Require sample retest |
| | | | | | expected qPCR | and |
| | | | | | function across | Alerts Operator (No |
| | | | | | linear range. | operator override) |
| | | | Evogenous Control: | Ct (Xeno ™) ± 1 | Inhibitors | |
| | | | Sample RNA Spike-in | | Sample Integrity | |
| | | | Endogenous Control | Ct (Ref) ± 1 | 1. Adequate | |
| | | | Reference Gene(1) | | Sample RNA | |
| | | | | | Input. | |
| | | | | | 2. Control for | |
| | | | | | variable RNA | |
| | | | | | Input. | |
| Step 3 | Biomarker | Process QC | Biomarker integrity | QC flag | Usability of each | |
| | Range Check | | | | Biomarker Value | |
| Step 4 | Apply QC and | Process QC | Analytical integrity | QC flag | Usability of each | |
| | Biomarker | | | | Biomarker Value | |
| | Range Flags | | | | | |
| Step 5 | Calculate | Quantitative | Endogenous Control | Absorbed dose | Estimated | Checks for intended |
| | Estimated | | | (Gy) | absorbed Dose | use dose range |
| | Dose | | | | | |
| | Calculate Dose | Measurement | Confidence Interval | Dose Range | Dose Interval | Checks for acceptable |
| | Interval | Confidence | | | | confidence interval |
| Step 6 | Combine | Report | Report | Gy | Dose Estimation | Clinic |
| | Estimated | | | | Report | Review/Approval |
| | Dose and | | | | | |
| | Dose Interval | | | | | |

In one embodiment, the target mRNAs or cDNAs to which the primers hybridize are those from the following (human) RM genes: CR2, DHRS4L1, HCK, IL1RAP, LYRM4, MYC, TMEM63B, ALOX5, CAMK4, CDKN1A, COCH, DHRS4, MICAL1, MOB3B, NUSAP1, IL27RA, HBA2, PPM1F, PPP2R1A, CFLAR, DHRS13, ACAA1, INPP5J, OAZ1, PNOC, PDE4B, SCARB1, and TMEM9B.

In other embodiments, mRNAs or cDNAs to which primers hybridize may include the following genes: ADAM17, AKT1, ANK1, ANXA3, ARHGAP26, ARID4A, ATG2A, ATIC, BCL11A, BCL6, BID, CFLAR, CIT, CPVL, CYTH4, DDB2, DDX58, DTL, EHBPL1, FCGR2A, FGR, HPRT1, HSP90AB1, HTRA2, IDOL, IL27RA, IRF1, JMJD1C, KIAA0101, LARP4B, LRRC6, LYN, MAP3K11, MAPK3, MDM1, MKNK1, MXD1, NAIP, NFE2L2, NRG1, NUSAP, PCNA, PGK1, PMP22, PPP2RA1, RARA, RNASE6, RPL13A, RPL6, RPS14, SCARB1, SP110, SPOCK2, TAPBP, TBP, TCF3, TNFRSF1A, TNFRSF1B, TNFSF14, USP38, WDR48, XAF1, ZAK, NPM1, ALAS2, ALPK1, CD97, CPSF1, COASY, CXXC5, DNAJC10, DYNLRB1, ELK4, ESD, GPR183, GPRIN, NDE1, PGS1, PPM1K, PTAFR, SLC6A6, SPECC1, and TEX10.

In some embodiments, primers are also included that hybridize to PPP6R3 mRNA or cDNA, where PPP6R3 and its mRNA levels serve as a reference gene for relative quantification of RM gene expression levels in an amplification reaction. In other embodiments primers may be included that hybridize to USP38, WDR48 or LARP4B mRNA or cDNA to serve as the reference gene or some combination thereof.

In some embodiments, the nucleic acid amplification reactions are qPCR reactions. In some embodiments the qPCR reactions are TAQMAN® probe qPCR reactions that include, in addition to the target primer pairs, TAQMAN® probes that hybridize under stringent conditions to the RM gene or reference gene mRNAs or cDNAs. TAQMAN® probe-based qPCR assays are well known in the art as described in, e.g., U.S. Pat. Nos. 5,677,152, 5,773,258 and 5,804,375.

Exemplary RM and reference gene primer and TAQMAN® probe sequences are listed below in Table 3.

TABLE 3
RM and reference gene primer
and TAQMAN ® probe sequences.

| | | Amplicon | Length | |
|---|---|---|---|---|
| Gene | Assay ID | Sequence | (bp) | Tm |
| PPP6R3 | Hs00217759_ml | TGAGGGAGGAAG | 75 | 55-65° C. |
| | | ACGGCATGGTTA | | |
| | | CATGGGACACCT | | |
| | | AACGAGGATAGC | | |
| | | TAACTGTATCGT | | |
| | | GCACAGCACTGA | | |
| | | CAAG | | |
| | | (SEQ ID | | |
| | | NO: 30) | | |
| CDKN1A | Hs00217759_ml | GACAGATTTCTA | 63 | 55-65° C. |
| | | CCACTCCAAACG | | |
| | | CCGGCTGATCTT | | |
| | | CTCCAAGAGGAA | | |
| | | GCCCTAATCCGC | | |
| | | CCACAG | | |
| | | (SEQ ID | | |
| | | NO: 31) | | |

Typically, stringent hybridization reaction conditions are defined by use of TAQPATH™ qPCR Mastermix chemistry and cycling conditions listed below in Table 4.

TABLE 4
Thermal Cycling Conditions for Target/
Primer/Probe Hybridization.

| | | | | | PCR | | | |
|---|---|---|---|---|---|---|---|---|
| | | | | | Cycle (40 cycles) | | | |
| | Incubation | | Activation | | | | Anneal/ | |
| Step | Hold | | Hold | | Denature | | Extend | |
| Temperature | 50° | C. | 95° | C. | 95° | C. | 60° | C. |
| Time | 2 | min. | 20 | sec. | 1 | sec. | 20 | sec. |

| | |
|---|---|
| Volume | 10 μL |

In some embodiments the plurality of nucleic acid amplification reactions are multiplexed such that multiple mRNAs (including a reference mRNA) are assayed in a single qPCR reaction, i.e., nine qPCR reactions would be needed to assay the entire panel of RM gene mRNAs from one sample, where each of the reactions are “tetraplexed,” 14 reactions would be needed per sample where each reaction is “triplexed”, and 28 reactions would be needed per sample when each qPCR reactions includes primers to a single RM gene mRNA and a reference gene mRNA. In some embodiments, the plurality of qPCR reactions can include different multiplexing, i.e., some reactions may contain primer pairs directed to three RM gene mRNAs and others a primer pair to only two or a single RM gene mRNA. The plurality of reactions can be provided in a number of formats, e.g., 96-, 384-, or even 1536-well formats.

In various embodiments, the mRNA or cDNA in the biodosimetry assay system is from a biological sample from a subject subjected to radiation exposure from about 30 minutes to about seven days prior to the time point at which the biological sample was obtained from the subject, e.g., one hour, three hours, 4 hours, six hours, twelve hours, 1 day, 2 days, 3 days, 4 days, 5 days, 7 days or another time period before biological sample collection from the subject ranging from about 30 minutes to about seven days.

Also contemplated herein is a radiation biomarker assay kit that includes a nucleic acid probe set consisting essentially of nucleic acid probes that hybridize specifically with nucleic acid targets comprising at least one of SEQ ID NOS: 1-29 or the complementary sequences thereof.

In some embodiments the probe set includes no more than about 200 probes, e.g., PCR primers. In other embodiments the probe set includes no more than about 100 probes.

In some embodiments the nucleic acid probe set includes primer pairs and TAQMAN® probes suitable for qPCR analysis of mRNAs or cDNAs comprising at least one of SEQ ID NOS: 1-29.

In some embodiments the kit also includes a thermostable polymerase suitable for qPCR, e.g., Taq polymerase and variants thereof known in the art.

In some embodiments a qPCR probe set in the kit is provided in a multi-well plate format. In some a multi-well plate is provided in which at least two nucleic acid probes that hybridize to at least two different nucleic acid targets are in the same wells, i.e., the probes can be multiplexed, as described above such that up to four different targets can be assayed by qPCR in the same reaction.

In some embodiments the kit also includes radiation exposure positive and negative control mRNA samples, which ensure that a qPCR biodosimetry assay is working properly, i.e., modulation of RM gene expression is detected in the positive control sample and no modulation of RM gene expression is detected in the negative control sample.

### EXAMPLES

The invention will be more fully understood upon consideration of the following non-limiting Examples. The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

Example 1: Description of Test Experiments used to Develop Biomarkers.

Rhesus macaque non-human primate (NHP) in vivo testing was conducted to produce single-dose biodosimetry samples and age/gender confounded samples to calibrate the biodosimeter.

NHP in vivo dose response to radiation: The animal test laboratory completed NHP Cobalt-60 irradiations at 0, 2, 4, and 6 (LD30/60), 7 Gy (LD70/60), and 10 Gy with cohorts of 16 (8 male and 8 female), at dose rate of approximately 0.6 Gy/min. Samples of 2.5 ml peripheral blood (PAXGENE™M blood RNA tube) were obtained from each rhesus macaque −2 week and −24 hr. prior to irradiation and 4 hr., 24 hr., 36 hr. post radiation, and on days 2, 3, 5, and 7 for a total of 9 blood draws per animal. Samples (0.5 ml) were also obtained in EDTA tubes to determine WBC differentials. Tests were staged to provide 4 NHP at each condition to determine target genes using discovery techniques (Phase 1), 10 NHP at each condition to determine biomarkers and 2 NHP at each condition to test the biodosimeter (algorithm) accuracy (Phase 2).

NHP confounder analysis; old age and juvenile. The animal test laboratory completed testing of 4 rhesus macaques (2 male and 2 female) exposed to 6 Gy (LD30/60) at a dose rate of approximately 0.6 Gy/min for both geriatric (>15 years) and juvenile (10-14 months) cohorts. Samples of 2.5 ml peripheral blood (PAXGENE™ blood RNA tube) were collected from each NHP −2 week and −24 hr. prior to irradiation and 4 hr., 24 hr., 36 hr. post radiation, and on days 2, 3, 5, and 7 for a total of 9 blood draws per animal. Samples (0.5 ml) were also obtained in EDTA tubes to determine WBC differentials.

NHP Fractionated Dose Testing: Two NHP models were developed to compare NHP gene response to human gene response for fractionated dose radiotherapy models.

NHP Fractionated Dose Models: Blood (2.5 ml) was collected from 6 female and 6 male rhesus NHP into PAXGENE™ blood RNA tubes. The NHP were irradiated in vivo to parallel the 4 human in vivo test protocols as described below. For Study 1: Twelve (12) NHPs were exposed to 1.5 Gy twice per day (dose rate 0.6-0.8 Gy/min.) for 4 days at the same time each day. The blood samples were collected within 24 hr. prior to irradiation and 24 hrs. after each daily exposure (6 draws). For Study 2: Twelve (12) NHPs were exposed to 1.2 Gy (dose rate 0.6-0.8 Gy/min.) 3 times per day at the same time each day for 4-days. Blood samples were collected prior to irradiation and 24 hr. following each exposure (prior to the next exposure) for a total of 6 draws. The NHPs were irradiated by LINAC. At the time of sample collection, a complete differential white cell count was conducted.

Human Fractionated Dose Models: Four human in vivo models were co-developed with Mayo Clinic, City of Hope and Stanford to provide blood samples from humans undergoing whole body and fractional radiation.

Model 1—Bone Marrow Transplant Patients (BMT): Radiation dose is 1.65 to 2 Gy twice daily for 3 to 4 days. Samples are taken prior to and 24 hr. after daily irradiations. The last draw is on Day-7; 7 days after the first dose. (4-6 samples/Series).

Model 2—Bone Marrow Transplant Patients (BMT): Radiation dose is 1.2 Gy three times daily for 4 days. Samples are taken prior to and 24 hr. after irradiation daily irradiations. The last draw is on Day-3 or 4; 3 or 4 days after the first dose. (5-7 samples/Series).

Model 3—Bone Marrow Transplant Patients (BMT): Radiation dose is a single fraction related to models 1 and 2. Samples are taken prior to and every 24 hr. after irradiation. The last draw is on Day-6; 6 days after the first dose. (6-7 samples/Series).

Model 4—X-Ray Therapy (XRT) Patients (>7% bone marrow exposure): Radiation dose is 2-8 Gy each day for multiple days. Samples are taken prior to and 24 hr. after irradiation. The last draw is taken 7 days after the last exposure.

TABLE 5
Nucleotide Sequences of Biodosimetry Biomarker Genes

CR2 (SEQ ID NO: 1; GenBank NM_001006658.2).
ATTTAAGGGCCCGCCTCTCCTGGCTCACAGCTGCTTGCTGCTCCAGCCTTGCCCTCCC
AGAGCTGCCGGACGCTCGCGGGTCTCGGAACGCATCCCGCCGCGGGGGCTTCGGCC
GTGGCATGGGCGCCGCGGGCCTGCTCGGGGTTTTCTTGGCTCTCGTCGCACCGGGGG
TCCTCGGGATTTCTTGTGGCTCTCCTCCGCCTATCCTAAATGGCCGGATTAGTTATTA
TTCTACCCCCATTGCTGTTGGTACCGTGATAAGGTACAGTTGTTCAGGTACCTTCCGC
CTCATTGGAGAAAAAAGTCTATTATGCATAACTAAAGACAAAGTGGATGGAACCTG
GGATAAACCTGCTCCTAAATGTGAATATTTCAATAAATATTCTTCTTGCCCTGAGCCC
ATAGTACCAGGAGGATACAAAATTAGAGGCTCTACACCCTACAGACATGGTGATTC
TGTGACATTTGCCTGTAAAACCAACTTCTCCATGAACGGAAACAAGTCTGTTTGGTG
TCAAGCAAATAATATGTGGGGGCCGACACGACTACCAACCTGTGTAAGTGTTTTCCC
TCTCGAGTGTCCAGCACTTCCTATGATCCACAATGGACATCACACAAGTGAGAATGT
TGGCTCCATTGCTCCAGGATTGTCTGTGACTTACAGCTGTGAATCTGGTTACTTGCTT
GTTGGAGAAAAGATCATTAACTGTTTGTCTTCGGGAAAATGGAGTGCTGTCCCCCCC
ACATGTGAAGAGGCACGCTGTAAATCTCTAGGACGATTTCCCAATGGGAAGGTAAA
GGAGCCTCCAATTCTCCGGGTTGGTGTAACTGCAAACTTTTTCTGTGATGAAGGGTA
TCGACTGCAAGGCCCACCTTCTAGTCGGTGTGTAATTGCTGGACAGGGAGTTGCTTG
GACCAAAATGCCAGTATGTGAAGAAATTTTTTGCCCATCACCTCCCCCTATTCTCAA
TGGAAGACATATAGGCAACTCACTAGCAAATGTCTCATATGGAAGCATAGTCACTTA
CACTTGTGACCCGGACCCAGAGGAAGGAGTGAACTTCATCCTTATTGGAGAGAGCA
CTCTCCGTTGTACAGTTGATAGTCAGAAGACTGGGACCTGGAGTGGCCCTGCCCCAC
GCTGTGAACTTTCTACTTCTGCGGTTCAGTGTCCACATCCCCAGATCCTAAGAGGCC
GAATGGTATCTGGGCAGAAAGATCGATATACCTATAACGACACTGTGATATTTGCTT
GCATGTTTGGCTTCACCTTGAAGGGCAGCAAGCAAATCCGATGCAATGCCCAAGGC
ACATGGGAGCCATCTGCACCAGTCTGTGAAAAGGAATGCCAGGCCCCTCCTAACAT
CCTCAATGGGCAAAAGGAAGATAGACACATGGTCCGCTTTGACCCTGGAACATCTA
TAAAATATAGCTGTAACCCTGGCTATGTGCTGGTGGGAGAAGAATCCATACAGTGTA
CCTCTGAGGGGGTGTGGACACCCCCTGTACCCCAATGCAAAGTGGCAGCGTGTGAA
GCTACAGGAAGGCAACTCTTGACAAAACCCCAGCACCAATTTGTTAGACCAGATGT
CAACTCTTCTTGTGGTGAAGGGTACAAGTTAAGTGGGAGTGTTTATCAGGAGTGTCA
AGGCACAATTCCTTGGTTTATGGAGATTCGTCTTTGTAAAGAAATCACCTGCCCACC
ACCCCCTGTTATCTACAATGGGGCACACACCGGGAGTTCCTTAGAAGATTTTCCATA
TGGAACCACGGTCACTTACACATGTAACCCTGGGCCAGAAAGAGGAGTGGAATTCA
GCCTCATTGGAGAGAGCACCATCCGTTGTACAAGCAATGATCAAGAAAGAGGCACC
TGGAGTGGCCCTGCTCCCCTGTGTAAACTTTCCCTCCTTGCTGTCCAGTGCTCACATG
TCCATATTGCAAATGGATACAAGATATCTGGCAAGGAAGCCCCATATTTCTACAATG
ACACTGTGACATTCAAGTGTTATAGTGGATTTACTTTGAAGGGCAGTAGTCAGATTC
GTTGCAAAGCTGATAACACCTGGGATCCTGAAATACCAGTTTGTGAAAAAGGCTGC
CAGTCACCTCCTGGGCTCCACCATGGTCGTCATACAGGTGGAAATACGGTCTTCTTT
GTCTCTGGGATGACTGTAGACTACACTTGTGACCCTGGCTATTTGCTTGTGGGAAAC
AAATCCATTCACTGTATGCCTTCAGGAAATTGGAGTCCTTCTGCCCCACGGTGTGAA
GAAACATGCCAGCATGTGAGACAGAGTCTTCAAGAACTTCCAGCTGGTTCACGTGTG
GAGCTAGTTAATACGTCCTGCCAAGATGGGTACCAGTTGACTGGACATGCTTATCAG
ATGTGTCAAGATGCTGAAAATGGAATTTGGTTCAAAAAGATTCCACTTTGTAAAGTT
ATTCACTGTCACCCTCCACCAGTGATTGTCAATGGGAAGCACACAGGCATGATGGCA
GAAAACTTTCTATATGGAAATGAAGTCTCTTATGAATGTGACCAAGGATTCTATCTC
CTGGGAGAGAAAAAATTGCAGTGCAGAAGTGATTCTAAAGGACATGGATCTTGGAG
CGGGCCTTCCCCACAGTGCTTACGATCTCCTCCTGTGACTCGCTGCCCTAATCCAGA
AGTCAAACATGGGTACAAGCTCAATAAAACACATTCTGCATATTCCCACAATGACAT
AGTGTATGTTGACTGCAATCCTGGCTTCATCATGAATGGTAGTCGCGTGATTAGGTG
TCATACTGATAACACATGGGTGCCAGGTGTGCCAACTTGTATCAAAAAAGCCTTCAT
AGGGTGTCCACCTCCGCCTAAGACCCCTAACGGGAACCATACTGGTGGAAACATAG
CTCGATTTTCTCCTGGAATGTCAATCCTGTACAGCTGTGACCAAGGCTACCTGCTGGT
GGGAGAGGCACTCCTTCTTTGCACACATGAGGGAACCTGGAGCCAACCTGCCCCTC
ATTGTAAAGAGGTAAACTGTAGCTCACCAGCAGATATGGATGGAATCCAGAAAGGG
CTGGAACCAAGGAAAATGTATCAGTATGGAGCTGTTGTAACTCTGGAGTGTGAAGA
TGGGTATATGCTGGAAGGCAGTCCCCAGAGCCAGTGCCAATCGGATCACCAATGGA
ACCCTCCCCTGGCGGTTTGCAGATCCCGTTCACTTGCTCCTGTCCTTTGTGGTATTGC
TGCAGGTTTGATACTTCTTACCTTCTTGATTGTCATTACCTTATACGTGATATCAAAA
CACAGAGCACGCAATTATTATACAGATACAAGCCAGAAAGAAGCTTTTCATTTAGA
AGCACGAGAAGTATATTCTGTTGATCCATACAACCCAGCCAGCTGATCAGAAGACA
AACTGGTGTGTGCCTCATTGCTTGGAATTCAGCGGAATATTGATTAGAAAGAAACTG
CTCTAATATCAGCAAGTCTCTTTATATGGCCTCAAGATCAATGAAATGATGTCATAA
GCGATCACTTCCTATATGCACTTATTCTCAAGAAGAACATCTTTATGGTAAAGATGG
GAGCCCAGTTTCACTGCCATATACTCTTCAAGGACTTTCTGAAGCCTCACTTATGAG
ATGCCTGAAGCCAGGCCATGGCTATAAACAATTACATGGCTCTAAAAAGTTTTGCCC
TTTTTAAGGAAGGCACTAAAAAGAGCTGTCCTGGTATCTAGACCCATCTTCTTTTTG
AAATCAGCATACTCAATGTTACTATCTGCTTTTGGTTATAATGTGTTTTTAATTATCT
AAAGTATGAAGCATTTTCTGGGGTTATGATGGCTTTACCTTTATTAGGAAGTATGGT
TTTATTTTGATAGTAGCTTCCTCCTCTGGTGGTGTTAATCATTTCATTTTTACCCTTAC
TTGGTTTGAGTTTCTCTCACATTACTGTATATACTTTGCCTTTCCATAATCACTCAGTG
ATTGCAATTTGCACAAGTTTTTTTAAATTATGGGAATCAAGATTTAATCCTAGAGATT
TGGTGTACAATTCAGGCTTTGGATGTTTCTTTAGCAGTTTTGTGATAAGTTCTAGTTG
CTTGTAAAATTTCACTTAATAATGTGTACATTAGTCATTCAATAAATTGTAATTGTAA
AGAAAACATACAAAAAAAAAAAAAAAA
DHRS4L1 (SEQ ID NO: 2; GenBank NM_001277864.1).
AGTCGGGCAGCTCTCCGGGCCGGCGTGGGAGCCCGCGCTCCAAAGCCCGGTGGGGG
GAGGGGCGCTCACGCAACCGCCACTGTCTGGAGCGGGCTCGCCTCTGCGGCGGCAC
TCACCGCCCGGGCTTTACTGAAGCGGAGTCTAGCATGTGCGGCTGCTCCACAGCGGT
GTGGGTGGCGGCGGCTCCTCTGCAGCAGCCTCGGCAGTAGGGGTCACGGTGGCCAA
GCCCACCGTGGAGCTCATCTGAGAGTTGTAAGGTACGGGACTGCCTCGGTCTTTGGG
ACGCCCCGTCTGGTAGCATCCCAGATCCAGCACGTTCCTTCCGGCCCTGCACCCCGG
CCCGGTGCCTCACACCCCGCTACCCCATGCATCCAGACTCTAAGGCAGCCCCTGCAT
CTCAGTCCTGACATCGCTGTCCCTGGAGCATCCTCCGCTGGAGCTGGAGCTTGACAG
GATCGGCTTCGCCGTCGCCCAGCGTCTGGCCCAAGACGGGGCCCACGTGGTAGTCA
GCCGCCGGAAGCAGCAGAATGTGGACCAGGCAGTGGCCACGCTGCAGGGGGAGGG
GCTGAGCATGACGGGCACTGTGTGCCATGTGGGGAAGATGAAGGACTGGGAGCGGC
TGGTGGCCACAGTGAGCTGCAGGGAAATGGGCACAGAGCCAGGAGGTGGAAAAGG
GAGCCAGCCTGAGCCTCCTTCCCTGCTTTCCTGGACAGCATTGGGCTTCAGTCCTTAC
AATGTCAGTAAAACAGCCTTGCTGGGCCTCAACAAGACCTTGGCCATAGAGCTGGC
CCCAAGGAACATTAGGGTGAACTGCCTAGCACCTGGACTTATCAAGACTAGCTTCAG
CAGGATGCTCTGGATGGACAAGGAAAAAGAGGAAAGCATGAAAGAAACCCTGCGG
ATAAGAAGGTTAGGCGAGCCAGAGGATTCTCTTGGCATCGTGTCTTTCCTGTGCTCT
GAAGATGCCAGCTACCTCACTGGGGAAACAGTGATGGTGGGTGGAGGAACCCCGTC
CCGCCTCTGAGGACCCGGAGACAGCCCACAGGCCAGAGTTGGGCTCTAGCTCCTGG
TGCTGTTCCTGCATTCACCCACTGGCCTTTCCCACCTCTGCTCACCTTACTGTTCACC
TCATCAAATCAGTTCTGCCCTGTGAAAAGATCCAGCCTTCCCTGCCGTCAAGGTGGT
GTCTTACTCGGGATTCCTGCTGTTGTTGTGGCCTTGGGTAAAGGCCTCCCCTGAGAA
CACAGGACAGGCCTGCTGACAAGGCTGAGTCTACCTTGGCAAAGACCAAGATATTT
TTTGCCCAGGCCACTGGGGAATTTGAGGGGAGATGAGAGAGAAGGAAGCTGGAGTG
GAAGGAGCAGAGTTGCAAATTAACAACTTGCAAATGAGGTGCAAATAAAATGCAGA
TGATTGCGCGGCTTTGAATCGAAAAAAAAAAA
HCK (SEQ ID NO: 3; GenBank NM_001172129.1).
GGAGTTAGCCTCGCTCAGGGCGCGGCTAAGGCGCCCAGATGGCCTGCGGGCGCCAC
CACGTCCCTGGTCCCAGCTCGGGAGCACATCAGAGGCTTAGAGGCGAGTGGGAAGG
GACTCAGACAGTGCAGGACGAGAAACGCCCGCGGCACCAAAGCCCCTCAGAGCGTC
GCCCCCGCCTCTAGTTCTAGAAAGTCAGTTTCCCGGCACTGGCACCCCGGAACCTCA
GGGGCTGCCGAGCTGGGGGGGCGCTCAAGCTGCGAGGATCCGGGCTGCCCGCGAGA
CGAGGAGCGGGCGCCCAGGATGGGGTGCATGAAGTCCAAGTTCCTCCAGGTCGGAG
GCAATACATTCTCAAAAACTGAAACCAGCGCCAGCCCACACTGTCCTGTGTACGTGC
CGGATCCCACATCCACCATCAAGCCGGGGCCTAATAGCCACAACAGCAACACACCA
GGAATCAGGGAGGCAGGCTCTGAGGACATCATCGTGGTTGCCCTGTATGATTACGA
GGCCATTCACCACGAAGACCTCAGCTTCCAGAAGGGGGACCAGATGGTGGTCCTAG
AGGAATCCGGGGAGTGGTGGAAGGCTCGATCCCTGGCCACCCGGAAGGAGGGCTAC
ATCCCAAGCAACTATGTCGCCCGCGTTGACTCTCTGGAGACAGAGGAGTGGTTTTTC
AAGGGCATCAGCCGGAAGGACGCAGAGCGCCAACTGCTGGCTCCCGGCAACATGCT
GGGCTCCTTCATGATCCGGGATAGCGAGACCACTAAAGGAAGCTACTCTTTGTCCGT
GCGAGACTACGACCCTCGGCAGGGAGATACCGTGAAACATTACAAGATCCGGACCC
TGGACAACGGGGGCTTCTACATATCCCCCCGAAGCACCTTCAGCACTCTGCAGGAGC
TGGTGGACCACTACAAGAAGGGGAACGACGGGCTCTGCCAGAAACTGTCGGTGCCC
TGCATGTCTTCCAAGCCCCAGAAGCCTTGGGAGAAAGATGCCTGGGAGATCCCTCG
GGAATCCCTCAAGCTGGAGAAGAAACTTGGAGCTGGGCAGTTTGGGGAAGTCTGGA
TGGCCACCTACAACAAGCACACCAAGGTGGCAGTGAAGACGATGAAGCCAGGGAG
CATGTCGGTGGAGGCCTTCCTGGCAGAGGCCAACGTGATGAAAACTCTGCAGCATG
ACAAGCTGGTCAAACTTCATGCGGTGGTCACCAAGGAGCCCATCTACATCATCACG
GAGTTCATGGCCAAAGGAAGCTTGCTGGACTTTCTGAAAAGTGATGAGGGCAGCAA
GCAGCCATTGCCAAAACTCATTGACTTCTCAGCCCAGATTGCAGAAGGCATGGCCTT
CATCGAGCAGAGGAACTACATCCACCGAGACCTCCGAGCTGCCAACATCTTGGTCTC
TGCATCCCTGGTGTGTAAGATTGCTGACTTTGGCCTGGCCCGGGTCATTGAGGACAA
CGAGTACACGGCTCGGGAAGGGGCCAAGTTCCCCATCAAGTGGACAGCTCCTGAAG
CCATCAACTTTGGCTCCTTCACCATCAAGTCAGACGTCTGGTCCTTTGGTATCCTGCT
GATGGAGATCGTCACCTACGGCCGGATCCCTTACCCAGGGATGTCAAACCCTGAAG
TGATCCGAGCTCTGGAGCGTGGATACCGGATGCCTCGCCCAGAGAACTGCCCAGAG
GAGCTCTACAACATCATGATGCGCTGCTGGAAAAACCGTCCGGAGGAGCGGCCGAC
CTTCGAATACATCCAGAGTGTGCTGGATGACTTCTACACGGCCACAGAGAGCCAGTA
CCAACAGCAGCCATGATAGGGAGGACCAGGGCAGGGCCAGGGGGTGCCCAGGTGG
TGGCTGCAAGGTGGCTCCAGCACCATCCGCCAGGGCCCACACCCCCTTCCTACTCCC
AGACACCCACCCTCGCTTCAGCCACAGTTTCCTCATCTGTCCAGTGGGTAGGTTGGA
CTGGAAAATCTCTTTTTGACTCTTGCAATCCACAATCTGACATTCTCAGGAAGCCCCC
AAGTTGATATTTCTATTTCCTGGAATGGTTGGATTTTAGTTACAGCTGTGATTTGGAA
GGGAAACTTTCAAAATAGTGAAATGAATATTTAAATAAAAGATATAAATGCCAAAG
TCTTTACCAAAAAAAAAAAAAAAAA
IL1RAP (SEQ ID NO: 4; GenBank NM_002182.3).
AAAGGGGGAAAAGAAAGTGCGGCGGAAAGTAAGAGGCTCACTGGGGAAGACTGCC
GGGATCCAGGTCTCCGGGGTCCGCTTTGGCCAGAGGCGCGGAAGGAAGCAGTGCCC
GGCGACACTGCACCCATCCCGGCTGCTTTTGCTGCGCCCTCTCAGCTTCCCAAGAAA
GGCATCGTCATGTGATCATCACCTAAGAACTAGAACATCAGCAGGCCCTAGAAGCC
TCACTCTTGCCCCTCCCTTTAATATCTCAAAGGATGACACTTCTGTGGTGTGTAGTGA
GTCTCTACTTTTATGGAATCCTGCAAAGTGATGCCTCAGAACGCTGCGATGACTGGG
GACTAGACACCATGAGGCAAATCCAAGTGTTTGAAGATGAGCCAGCTCGCATCAAG
TGCCCACTCTTTGAACACTTCTTGAAATTCAACTACAGCACAGCCCATTCAGCTGGC
CTTACTCTGATCTGGTATTGGACTAGGCAGGACCGGGACCTTGAGGAGCCAATTAAC
TTCCGCCTCCCCGAGAACCGCATTAGTAAGGAGAAAGATGTGCTGTGGTTCCGGCCC
ACTCTCCTCAATGACACTGGCAACTATACCTGCATGTTAAGGAACACTACATATTGC
AGCAAAGTTGCATTTCCCTTGGAAGTTGTTCAAAAAGACAGCTGTTTCAATTCCCCC
ATGAAACTCCCAGTGCATAAACTGTATATAGAATATGGCATTCAGAGGATCACTTGT
CCAAATGTAGATGGATATTTTCCTTCCAGTGTCAAACCGACTATCACTTGGTATATG
GGCTGTTATAAAATACAGAATTTTAATAATGTAATACCCGAAGGTATGAACTTGAGT
TTCCTCATTGCCTTAATTTCAAATAATGGAAATTACACATGTGTTGTTACATATCCAG
AAAATGGACGTACGTTTCATCTCACCAGGACTCTGACTGTAAAGGTAGTAGGCTCTC
CAAAAAATGCAGTGCCCCCTGTGATCCATTCACCTAATGATCATGTGGTCTATGAGA
AAGAACCAGGAGAGGAGCTACTCATTCCCTGTACGGTCTATTTTAGTTTTCTGATGG
ATTCTCGCAATGAGGTTTGGTGGACCATTGATGGAAAAAAACCTGATGACATCACTA
TTGATGTCACCATTAACGAAAGTATAAGTCATAGTAGAACAGAAGATGAAACAAGA
ACTCAGATTTTGAGCATCAAGAAAGTTACCTCTGAGGATCTCAAGCGCAGCTATGTC
TGTCATGCTAGAAGTGCCAAAGGCGAAGTTGCCAAAGCAGCCAAGGTGAAGCAGAA
AGTGCCAGCTCCAAGATACACAGTGGAACTGGCTTGTGGTTTTGGAGCCACAGTCCT
GCTAGTGGTGATTCTCATTGTTGTTTACCATGTTTACTGGCTAGAGATGGTCCTATTT
TACCGGGCTCATTTTGGAACAGATGAAACCATTTTAGATGGAAAAGAGTATGATATT
TATGTATCCTATGCAAGGAATGCGGAAGAAGAAGAATTTGTATTACTGACCCTCCGT
GGAGTTTTGGAGAATGAATTTGGATACAAGCTGTGCATCTTTGACCGAGACAGTCTG
CCTGGGGGAATTGTCACAGATGAGACTTTGAGCTTCATTCAGAAAAGCAGACGCCTC
CTGGTTGTTCTAAGCCCCAACTACGTGCTCCAGGGAACCCAAGCCCTCCTGGAGCTC
AAGGCTGGCCTAGAAAATATGGCCTCTCGGGGCAACATCAACGTCATTTTAGTACAG
TACAAAGCTGTGAAGGAAACGAAGGTGAAAGAGCTGAAGAGGGCTAAGACGGTGC
TCACGGTCATTAAATGGAAAGGGGAAAAATCCAAGTATCCACAGGGCAGGTTCTGG
AAGCAGCTGCAGGTGGCCATGCCAGTGAAGAAAAGTCCCAGGCGGTCTAGCAGTGA
TGAGCAGGGCCTCTCGTATTCATCTTTGAAAAATGTATGAAAGGAATAATGAAAAG
GGTAAAAAGAACAAGGGGTGCTCCAGGAAGAAAGAGTCCCCCCAGTCTTCATTCGC
AGTTTATGGTTTCATAGGCAAAAATAATGGTCTAAGCCTCCCAATAGGGATAAATTT
AGGGTGACTGTGTGGCTGACTATTCTGCTTCCTCAGGCAACACTAAAGTTTAGAAAG
ATATCATCAACGTTCTGTCACCAGTCTCTGATGCCACTATGTTCTTTGCAGGCAAAG
ACTTGTTCAATGCGAATTTCCCCTTCTACATTGTCTATCCCTGTTTTTATATGTCTCCA
TTCTTTTTAAAATCTTAACATATGGAGCAGCCTTTCCTATGAATTTAAATATGCCTTT
AAAATAAGTCACTGTTGACAGGGTCATGAGTTTCCGAGTATAGTTTTCTTTTTATCTT
ATTTTTACTCGTCCGTTGAAAAGATAATCAAGGCCTACATTTTAGCTGAGGATAATG
AACTTTTTTCCTCATTCGGCTGTATAATACATAACCACAGCAAGACTGACATCCACTT
AGGATGATACAAAGCAGTGTAACTGAAAATGTTTCTTTTAATTGATTTAAAGGACTT
GTCTTCTATACCACCCTTGTCCTCATCTCAGGTAATTTATGAAATCTATGTAAACTTG
AAAAATATTTCTTAATTTTTGTTTTTGCTCCAGTCAATTCCTGATTATCCACAGGTCA
ACCCACATTTTTTCATTCCTTCTCCCTATCTGCTTATATCGCATTGCTCATTTAGAGTT
TGCAGGAGGCTCCATACTAGGTTCAGTCTGAAAGAAATCTCCTAATGGTGCTATAGA
GAGGGAGGTAACAGAAAGACTCTTTTAGGGCATTTTTCTGACTCATGAAAAGAGCA
CAGAAAAGGATGTTTGGCAATTTGTCTTTTAAGTCTTAACCTTGCTAATGTGAATACT
GGGAAAGTGATTTTTTCTCACTCGTTTTTGTTGCTCCATTGTAAAGGGCGGAGGTCA
GTCTTAGTGGCCTTGAGAGTTGCTTTTGGCATTAATATTCTAAGAGAATTAACTGTAT
TTCCTGTCACCTATTCACTAGTGCAGGAAATATACTTGCTCCAAATAAGTCAGTATG
AGAAGTCACTGTCAATGAAAGTTGTTTTGTTTGTTTTCAGTAATATTTTGCTGTTTTT
AAGACTTGGAAAACTAAGTGCAGAGTTTACAGAGTGGTAAATATCTATGTTACATGT
AGATTATACATATATATACACACGTGTATATGAGATATATATCTTATATCTCCACAA
ACACAAATTATATATATACATATCCACACACATACATTACATATATCTGTGTATATA
AATCCACATGCACATGAAATATATATATATATATAATTTGTGTGTGTGTATGTGTAT
GTATATGACTTTAAATAGCTATGGGTACAATATTAAAAACCACTGGAACTCTTGTCC
AGTTTTTAAATTATGTTTTTACTGGAATGTTTTTGTGTCAGTGTTTTCTGTACATATTA
TTTGTTAATTCACAGCTCACAGAGTGATAGTTGTCATAGTTCTTGCCTTCCCTAAGTT
TATATAAATAACTTAAGTATTGCTACAGTTTATCTAGGTTGCAGTGGCATCTGCTGTG
CACAGAGCTTCCATGGTCACTGCTAAGCAGTAGCCAGCCATCGGGCATTAATTGATT
TCCTACTATATTCCCAGCAGACACATTTAGAAACTAAGCTATGTTAACCTCAGTGCT
CAACTATTTGAACTGTTGAGTGATAAAGGAAACAAATATAACTGTAAATGAATCTTG
GTATCCTGTGAAACAGAATAATTCGTAATTTAAGAAAGCCCTTATCCCGGTAACATG
AATGTTGATGAACAAATGTAAAATTATATCCTATATTTAAGTACCCATAATAAATCA
TTTCCCTCTATAAGTGTTATTGATTATTTTAAATTGAAAAAAGTTTCACTTGGATGAA
AAAAGTAGAAAAGTAGGTCATTCTTGGATCTACTTTTTTTTAGCCTTATTAATATTTT
TCCCTATTAGAAACCACAATTACTCCCTCTATTAACCCTTCACTTACTAGACCAGAA
AAGAACTTATTCCAGATAAGCTTTGAATATCAATTCTTACATAAACTTTAGGCAAAC
AGGGAATAGTCTAGTCACCAAAGGACCATTCTCTTGCCAATGCTGCATTCCTTTTGC
ACTTTTGGATTCCATATTTATCCCAAATGCTGTTGGGCACCCCTAGAAATACCTTGAT
GTTTTTTCTATTTATATGCCTGCCTTTGGTACTTAATTTTACAAATGCTGTAATATAA
AGCATATCAAGTTTATGTGATACGTATCATTGCAAGAGAATTTGTTTCAAGATTTTTT
TTTAATGTTCCAGAAGATGGCCAATAGAGAACATTCAAGGGAAATGGGGAAACATA
ATTTAGAGAACAAGAACAAACCATGTCTCAAATTTTTTTAAAAAAAATTAATGGTTT
TAAATATATGCTATAGGGACGTTCCATGCCCAGGTTAACAAAGAACTGTGATATATA
GAGTGTCTAATTACAAAATCATATACGATTTATTTAATTCTCTTCTGTATTGTAACTT
AGATGATTCCCAAGGACTCTAATAAAAAATCACTTCATTGTATTTGGAAACAAAAAC
ATCATTCATTAATTACTTATTTTCTTTCCATAGGTTTTAATATTTTGAGAGTGTCTTTT
TTATTTCATTCATGAACTTTTGTATTTTTCATTTTTCATTTGATTTGTAAATTTACTTAT
GTTAAAAATAAACCATTTATTTTCAGCTTTGAATTTTAAAAAAAAAAAAAAAAAA
LYRM4 (SEQ ID NO: 5; GenBank NM_020408.5).
GAGCCCTGCCTGCGCCCGCCCCCGAAGCGGCGCGGGACGCCTGGCGCCGTCCGCGA
TCCGCAGGGCTGCCCGCTTAGGCTTAGGCCCGGCCCGCTGGCAAAGCCGAGCCGCA
GCATTTTATTTCGTTCGTGGTTTCCGCACAGGCTGGAGTTTCGTGGGTTGGGTCGTAC
TTGGGACCTCGGCGAAGAGGACCCGTTTATTTTTTTTTCTTTCCAAAATGGCAGCCTC
CAGTCGCGCACAAGTGTTATCTCTGTACCGGGCGATGCTGAGAGAGAGCAAGCGTTT
CAGCGCCTACAATTACAGAACATATGCTGTCAGGAGGATAAGAGATGCCTTCAGAG
AAAATAAAAATGTAAAGGATCCTGTAGAAATTCAAACCCTAGTGAATAAAGCCAAG
AGAGACCTTGGAGTAATTCGTCGACAGGTCCACATTGGCCAACTGTATTCAACTGAC
AAGCTGATCATTGAGAATCGAGACATGCCCAGGACCTAGCAAGCCGGGGACCAGCC
ACCAGTGGCGGCCAGGGACCACCTTCAGCATCCACTCTCTGTTTGAGATGGGGGCTC
CCAAAACCAGCTTACAATAGCCTTTTGCGCTGCCTGTCCTGTGGGAGCTGATAAACC
AAGTCACATTTGCATTCTGTTGCAGGCTTAGTGAAAAAGGACTGCTGTCTTTCCTTG
GTTCAAGTGTTAGAATGGAGAGCTGGAGTTCGTTCAGAATAGTGCTGTGTGTTACCA
CGTCTCCCCTGCACCCCATTCCTACCTTGTAGCTCATGACCATTGTGTATAGCATTTC
TACACTTTGTTTCTTGGTCCTTGGCAATAAAAAGAATGATCTCCCTGAGCCTTTGACC
CCAGATAAACCCCTCCCAATTAATGCATTTTCATTTCCTACTGATACAAGGCCTGGA
GAGGGCTGTTGGGGGCCCTCAGGGAGGGTTCAACTCTGAGACGAGAACTGCCTTGG
TGAAGGCAAGTTCAAGCACCACTTGAGACTGGGGGCAGCATGGAGTAGGGCAGGGC
TACGGGGATACACGGTGCACCCTGCAACTTATACCTGAGCCCAGTACAACAAAGGT
GACGGGTGTGTAGGTACACACCCAGAGATGGAGCACTGCAGATCAGCAACCTCAGC
CCCACCTGGGAATTTGCTGGAAATGCAGGCTCAAGCCCCTCCCCACACCTGGTGAAT
GAGAGAGCCCCAGCCTGACCCAAGCCCAGGGCGACTCCCATACCCTGAAGCCTGGG
GCATGCTGGGCAGCACCGGTGCCCAAATCTGGCTGGTGGACAGAAGCACCTGGAGA
GTTGGAGAGCTTTTTAAAAAGACATCTCTCAGCACTTCCCTCTCTGCAGATTCTGACT
CAGTAAGTGAGGGGTGAGGCACAGTCATTTTTCTCTATTCTGAAGCTCTCCCACTGT
TTTCAATGTTTAACCAACTGGGGACCCCTGCTCTTTAAGTATATTACAGGTAATAAA
GATATTGTTTGTATGCTTTTAAAAAAAAAAAAAAAAAA.
MYC (SEQ ID NO: 6; GenBank NM_002467).
GACCCCCGAGCTGTGCTGCTCGCGGCCGCCACCGCCGGGCCCCGGCCGTCCCTGGCT
CCCCTCCTGCCTCGAGAAGGGCAGGGCTTCTCAGAGGCTTGGCGGGAAAAAGAACG
GAGGGAGGGATCGCGCTGAGTATAAAAGCCGGTTTTCGGGGCTTTATCTAACTCGCT
GTAGTAATTCCAGCGAGAGGCAGAGGGAGCGAGCGGGCGGCCGGCTAGGGTGGAA
GAGCCGGGCGAGCAGAGCTGCGCTGCGGGCGTCCTGGGAAGGGAGATCCGGAGCG
AATAGGGGGCTTCGCCTCTGGCCCAGCCCTCCCGCTGATCCCCCAGCCAGCGGTCCG
CAACCCTTGCCGCATCCACGAAACTTTGCCCATAGCAGCGGGCGGGCACTTTGCACT
GGAACTTACAACACCCGAGCAAGGACGCGACTCTCCCGACGCGGGGAGGCTATTCT
GCCCATTTGGGGACACTTCCCCGCCGCTGCCAGGACCCGCTTCTCTGAAAGGCTCTC
CTTGCAGCTGCTTAGACGCTGGATTTTTTTCGGGTAGTGGAAAACCAGCAGCCTCCC
GCGACGATGCCCCTCAACGTTAGCTTCACCAACAGGAACTATGACCTCGACTACGAC
TCGGTGCAGCCGTATTTCTACTGCGACGAGGAGGAGAACTTCTACCAGCAGCAGCA
GCAGAGCGAGCTGCAGCCCCCGGCGCCCAGCGAGGATATCTGGAAGAAATTCGAGC
TGCTGCCCACCCCGCCCCTGTCCCCTAGCCGCCGCTCCGGGCTCTGCTCGCCCTCCTA
CGTTGCGGTCACACCCTTCTCCCTTCGGGGAGACAACGACGGCGGTGGCGGGAGCTT
CTCCACGGCCGACCAGCTGGAGATGGTGACCGAGCTGCTGGGAGGAGACATGGTGA
ACCAGAGTTTCATCTGCGACCCGGACGACGAGACCTTCATCAAAAACATCATCATCC
AGGACTGTATGTGGAGCGGCTTCTCGGCCGCCGCCAAGCTCGTCTCAGAGAAGCTG
GCCTCCTACCAGGCTGCGCGCAAAGACAGCGGCAGCCCGAACCCCGCCCGCGGCCA
CAGCGTCTGCTCCACCTCCAGCTTGTACCTGCAGGATCTGAGCGCCGCCGCCTCAGA
GTGCATCGACCCCTCGGTGGTCTTCCCCTACCCTCTCAACGACAGCAGCTCGCCCAA
GTCCTGCGCCTCGCAAGACTCCAGCGCCTTCTCTCCGTCCTCGGATTCTCTGCTCTCC
TCGACGGAGTCCTCCCCGCAGGGCAGCCCCGAGCCCCTGGTGCTCCATGAGGAGAC
ACCGCCCACCACCAGCAGCGACTCTGAGGAGGAACAAGAAGATGAGGAAGAAATC
GATGTTGTTTCTGTGGAAAAGAGGCAGGCTCCTGGCAAAAGGTCAGAGTCTGGATC
ACCTTCTGCTGGAGGCCACAGCAAACCTCCTCACAGCCCACTGGTCCTCAAGAGGTG
CCACGTCTCCACACATCAGCACAACTACGCAGCGCCTCCCTCCACTCGGAAGGACTA
TCCTGCTGCCAAGAGGGTCAAGTTGGACAGTGTCAGAGTCCTGAGACAGATCAGCA
ACAACCGAAAATGCACCAGCCCCAGGTCCTCGGACACCGAGGAGAATGTCAAGAGG
CGAACACACAACGTCTTGGAGCGCCAGAGGAGGAACGAGCTAAAACGGAGCTTTTT
TGCCCTGCGTGACCAGATCCCGGAGTTGGAAAACAATGAAAAGGCCCCCAAGGTAG
TTATCCTTAAAAAAGCCACAGCATACATCCTGTCCGTCCAAGCAGAGGAGCAAAAG
CTCATTTCTGAAGAGGACTTGTTGCGGAAACGACGAGAACAGTTGAAACACAAACT
TGAACAGCTACGGAACTCTTGTGCGTAAGGAAAAGTAAGGAAAACGATTCCTTCTA
ACAGAAATGTCCTGAGCAATCACCTATGAACTTGTTTCAAATGCATGATCAAATGCA
ACCTCACAACCTTGGCTGAGTCTTGAGACTGAAAGATTTAGCCATAATGTAAACTGC
CTCAAATTGGACTTTGGGCATAAAAGAACTTTTTTATGCTTACCATCTTTTTTTTTTCT
TTAACAGATTTGTATTTAAGAATTGTTTTTAAAAAATTTTAAGATTTACACAATGTTT
CTCTGTAAATATTGCCATTAAATGTAAATAACTTTAATAAAACGTTTATAGCAGTTA
CACAGAATTTCAATCCTAGTATATAGTACCTAGTATTATAGGTACTATAAACCCTAA
TTTTTTTTATTTAAGTACATTTTGCTTTTTAAAGTTGATTTTTTTCTATTGTTTTTAGAA
AAAATAAAATAACTGGCAAATATATCATTGAGCCAAATCTTAAAAAAAAAAAAAAA
TMEM63B (SEQ ID NO: 7; GenBank NM_018426.1).
AACCCGGGGCTCCGAGCCGGAGCCGAGTCTGCGCCTGGGGGAGGACCATGCGGCAG
TAGCAGCCATGCTGCCCTTTCTGCTGGCCACACTGGGCACCACAGCCCTCAACAACA
GCAACCCCAAGGACTACTGCTACAGCGCCCGCATCCGCAGCACTGTCCTGCAGGGC
CTGCCCTTTGGGGGCGTCCCCACCGTGCTGGCTCTCGACTTCATGTGCTTCCTTGCAC
TGCTGTTCTTATTCTCTATCCTCCGGAAGGTGGCCTGGGACTATGGGCGGCTGGCCTT
GGTGACAGATGCAGACAGGCTTCGGCGGCAGGAGAGGGACCGAGTGGAACAGGAA
TATGTGGCTTCAGCTATGCACGGGGACAGCCATGACCGGTATGAGCGTCTCACCTCT
GTCTCCAGCTCCGTTGACTTTGACCAAAGGGACAATGGTTTCTGTTCCTGGCTGACA
GCCATCTTCAGGATAAAGGATGATGAGATCCGGGACAAATGTGGGGGCGATGCCGT
GCACTACCTGTCCTTTCAGCGGCACATCATCGGGCTGCTGGTGGTTGTGGGCGTCCT
CTCCGTAGGCATCGTGCTGCCTGTCAACTTCTCAGGGGACCTGCTGGAGAACAATGC
CTACAGCTTTGGGAGAACCACCATTGCCAACTTGAAATCAGGGAACAACCTGCTATG
GCTGCACACCTCCTTCGCCTTCCTGTATCTGCTGCTCACCGTCTACAGCATGCGTAGA
CACACCTCCAAGATGCGCTACAAGGAGGATGATCTGGTGAAGCGGACCCTCTTCAT
CAATGGAATCTCCAAATATGCAGAGTCAGAAAAGATCAAGAAGCATTTTGAGGAAG
CCTACCCCAACTGCACAGTTCTCGAAGCCCGCCCGTGTTACAACGTGGCTCGCCTAA
TGTTCCTCGATGCAGAGAGGAAGAAGGCCGAGCGGGGAAAGCTGTACTTCACAAAC
CTCCAGAGCAAGGAGAACGTGCCTACCATGATCAACCCCAAGCCCTGTGGCCACCT
CTGCTGCTGTGTGGTGCGAGGCTGTGAGCAGGTGGAGGCCATTGAGTACTACACAA
AGCTGGAGCAGAAGCTGAAGGAAGACTACAAGCGGGAGAAGGAGAAGGTGAATGA
GAAGCCTCTTGGCATGGCCTTTGTCACCTTCCACAATGAGACTATCACCGCCATCAT
CCTGAAGGACTTCAACGTGTGTAAATGCCAGGGCTGCACCTGCCGTGGGGAGCCAC
GCCCCTCATCCTGCAGCGAGTCCCTGCACATCTCCAACTGGACCGTGTCCTATGCCC
CTGACCCTCAGAACATCTACTGGGAGCACCTCTCCATCCGAGGCTTCATCTGGTGGC
TGCGCTGCCTGGTCATCAATGTCGTCCTCTTCATCCTCCTCTTCTTCCTCACCACTCC
AGCCATCATCATCACCACCATGGACAAGTTCAACGTCACCAAGCCTGTGGAGTACCT
CAACAACCCCATCATCACCCAGTTCTTCCCCACCCTGCTGCTGTGGTGCTTCTCGGCC
CTCCTTCCCACCATCGTCTACTACTCAGCCTTCTTTGAAGCCCACTGGACACGCTCTG
GGGAGAACAGGACAACCATGCACAAGTGCTACACTTTCCTCATCTTCATGGTGCTGC
TCCTACCCTCGCTGGGACTGAGCAGCCTGGACCTCTTCTTCCGCTGGCTCTTTGATAA
GAAATTCTTGGCTGAGGCAGCTATTCGGTTTGAGTGTGTGTTCCTGCCCGACAACGG
CGCCTTCTTCGTGAACTACGTCATTGCCTCAGCCTTTATCGGCAACGCCATGGACCT
GCTGCGCATCCCAGGCCTGCTCATGTACATGATCCGGCTCTGCCTGGCGCGCTCGGC
CGCCGAGAGGCGCAACGTGAAGCGGCATCAGGCCTACGAGTTCCAGTTTGGCGCAG
CCTACGCCTGGATGATGTGCGTCTTCACGGTGGTCATGACCTACAGTATCACCTGCC
CCATCATCGTGCCCTTCGGGCTCATGTACATGCTGCTGAAGCACCTGGTAGACAGGT
ACAATCTCTACTACGCCTACCTGCCGGCCAAGCTGGACAAGAAGATCCACTCGGGG
GCTGTGAACCAGGTGGTGGCCGCGCCCATCCTCTGCCTCTTCTGGCTGCTCTTCTTTT
CCACCATGCGCACGGGGTTCCTAGCTCCCACGTCTATGTTCACATTTGTGGTCCTGGT
CATCACCATCGTCATCTGTCTCTGCCACGTCTGCTTTGGACACTTCAAATACCTCAGT
GCCCACAACTACAAGATTGAGCACACGGAGACAGATACTGTGGACCCCAGAAGCAA
TGGACGGCCCCCCACTGCTGCTGCTGTCCCCAAATCTGCGAAATACATCGCTCAGGT
GCTGCAGGACTCAGAGGTGGACGGGGATGGGGATGGGGCTCCTGGGAGCTCAGGGG
ATGAGCCCCCATCATCCTCATCCCAAGATGAGGAGTTGCTGATGCCACCCGACGCCC
TCACGGACACAGACTTCCAGTCTTGCGAGGACAGCCTCATAGAGAATGAGATTCAC
CAGTAAGGGGAGGGAGGGGCCCTGGAGGCCACATCCTGCCCCACCCCACCCCCACT
CCCACGGACACTAAAACGCTAATAATTTATTAGATCTAAAGCCCCTTCCTCCCCAGC
CCCTGCTTTCATTAAGGTATTTAAACTTGGGGGTTTCACTGCTCTCCCCCATGATGGA
GGGAGGGAGCCCCCCAACCTCAGTGAGGAGAGCCCCGAGCCGGCCCCGGGGCAAA
GAGGGGTGCAGAGGGAGTTCCCCCAGATCAGTACCCCCCACCCCTCCCCAGCTAGT
AGCATGACCAGGAGAGGGTTAATGAGAGCCAAGAGGAGTACCTGGTGCACCTGGTG
CCGGTGGCTGGAGACCTGGGGGGCAGGTGGATCTGGGGCTGTTCCCCCCCCTCCGTT
TTTTCCACCCCACAGTTCCTCCTGGGATCTGGCCCTCCAGGGAAGTGGAGCCTCCAG
CCCCTAGGGGATGCATGAGGGGGGAGGGGGTGCTGAGTGGGAGGAAGAGTCAGGC
TCACAGCTGGGGTGGCCTGGGGGTGGGGGTGGGCAAGGCTGACACTGGAAAATGGG
TTTTTGCACTGTTTTTTTTTTGGTTTTTTTGTTCTTTTTTGTTTTTTTCCTTTAAAATAA
AAACAAAGAAAAGCTCTGAAAAAAAAAAAAAAAAA
ALOX5 (SEQ ID NO: 8; GenBank NM_000698.3).
CCGGGGCCAGGGACCAGTGGTGGGAGGAGGCTGCGGCGCTAGATGCGGACACCTG
GACCGCCGCGCCGAGGCTCCCGGCGCTCGCTGCTCCCGCGGCCCGCGCCATGCCCTC
CTACACGGTCACCGTGGCCACTGGCAGCCAGTGGTTCGCCGGCACTGACGACTACAT
CTACCTCAGCCTCGTGGGCTCGGCGGGCTGCAGCGAGAAGCACCTGCTGGACAAGC
CCTTCTACAACGACTTCGAGCGTGGCGCGGTGGATTCATACGACGTGACTGTGGACG
AGGAACTGGGCGAGATCCAGCTGGTCAGAATCGAGAAGCGCAAGTACTGGCTGAAT
GACGACTGGTACCTGAAGTACATCACGCTGAAGACGCCCCACGGGGACTACATCGA
GTTCCCCTGCTACCGCTGGATCACCGGCGATGTCGAGGTTGTCCTGAGGGATGGACG
CGCAAAGTTGGCCCGAGATGACCAAATTCACATTCTCAAGCAACACCGACGTAAAG
AACTGGAAACACGGCAAAAACAATATCGATGGATGGAGTGGAACCCTGGCTTCCCC
TTGAGCATCGATGCCAAATGCCACAAGGATTTACCCCGTGATATCCAGTTTGATAGT
GAAAAAGGAGTGGACTTTGTTCTGAATTACTCCAAAGCGATGGAGAACCTGTTCATC
AACCGCTTCATGCACATGTTCCAGTCTTCTTGGAATGACTTCGCCGACTTTGAGAAA
ATCTTTGTCAAGATCAGCAACACTATTTCTGAGCGGGTCATGAATCACTGGCAGGAA
GACCTGATGTTTGGCTACCAGTTCCTGAATGGCTGCAACCCTGTGTTGATCCGGCGC
TGCACAGAGCTGCCCGAGAAGCTCCCGGTGACCACGGAGATGGTAGAGTGCAGCCT
GGAGCGGCAGCTCAGCTTGGAGCAGGAGGTCCAGCAAGGGAACATTTTCATCGTGG
ACTTTGAGCTGCTGGATGGCATCGATGCCAACAAAACAGACCCCTGCACACTCCAGT
TCCTGGCCGCTCCCATCTGCTTGCTGTATAAGAACCTGGCCAACAAGATTGTCCCCA
TTGCCATCCAGCTCAACCAAATCCCGGGAGATGAGAACCCTATTTTCCTCCCTTCGG
ATGCAAAATACGACTGGCTTTTGGCCAAAATCTGGGTGCGTTCCAGTGACTTCCACG
TCCACCAGACCATCACCCACCTTCTGCGAACACATCTGGTGTCTGAGGTTTTTGGCA
TTGCAATGTACCGCCAGCTGCCTGCTGTGCACCCCATTTTCAAGCTGCTGGTGGCAC
ACGTGAGATTCACCATTGCAATCAACACCAAGGCCCGTGAGCAGCTCATCTGCGAG
TGTGGCCTCTTTGACAAGGCCAACGCCACAGGGGGCGGTGGGCACGTGCAGATGGT
GCAGAGGGCCATGAAGGACCTGACCTATGCCTCCCTGTGCTTTCCCGAGGCCATCAA
GGCCCGGGGCATGGAGAGCAAAGAAGACATCCCCTACTACTTCTACCGGGACGACG
GGCTCCTGGTGTGGGAAGCCATCAGGACGTTCACGGCCGAGGTGGTAGACATCTAC
TACGAGGGCGACCAGGTGGTGGAGGAGGACCCGGAGCTGCAGGACTTCGTGAACGA
TGTCTACGTGTACGGCATGCGGGGCCGCAAGTCCTCAGGCTTCCCCAAGTCGGTCAA
GAGCCGGGAGCAGCTGTCGGAGTACCTGACCGTGGTGATCTTCACCGCCTCCGCCCA
GCACGCCGCGGTCAACTTCGGCCAGTACGACTGGTGCTCCTGGATCCCCAATGCGCC
CCCAACCATGCGAGCCCCGCCACCGACTGCCAAGGGCGTGGTGACCATTGAGCAGA
TCGTGGACACGCTGCCCGACCGCGGCCGCTCCTGCTGGCATCTGGGTGCAGTGTGGG
CGCTGAGCCAGTTCCAGGAAAACGAGCTGTTCCTGGGCATGTACCCAGAAGAGCAT
TTTATCGAGAAGCCTGTGAAGGAAGCCATGGCCCGATTCCGCAAGAACCTCGAGGC
CATTGTCAGCGTGATTGCTGAGCGCAACAAGAAGAAGCAGCTGCCATATTACTACTT
GTCCCCAGACCGGATTCCGAACAGTGTGGCCATCTGAGCACACTGCCAGTCTCACTG
TGGGAAGGCCAGCTGCCCCAGCCAGATGGACTCCAGCCTGCCTGGCAGGCTGTCTG
GCCAGGCCTCTTGGCAGTCACATCTCTTCCTCCGAGGCCAGTACCTTTCCATTTATTC
TTTGATCTTCAGGGAACTGCATAGATTGATCAAAGTGTAAACACCATAGGGACCCAT
TCTACACAGAGCAGGACTGCACAGCGTCCTGTCCACACCCAGCTCAGCATTTCCACA
CCAAGCAGCAACAGCAAATCACGACCACTGATAGATGTCTATTCTTGTTGGAGACAT
GGGATGATTATTTTCTGTTCTATTTGTGCTTAGTCCAATTCCTTGCACATAGTAGGTA
CCCAATTCAATTACTATTGAATGAATTAAGAATTGGTTGCCATAAAAATAAATCAGT
TCATTTAAAATGAAAAAAAAAAAAAAAAAAAA
CAMK4 (SEQ ID NO: 9; GenBank NM_001744.4).
AGTCTCCCTCCAGCGGGCGGCGACTCCGGGTTCCCCCTCGCGCCCTCTCGCAGAGGC
TCGCCCCCTTCCCCGCCCACCGTCCCTGCGAGCGCGGGCGGCGGCGGTGGGCGTGTG
CGCGCGTGAAGGACGCCGCCTCTCTCTCGCTCCTGCGTTCGCAGGCGGCGGCTGGCG
GCCGGCTTCTCGCTCGGGCAGCGGCGGCGGCGGCGGCGGCGGCTTCCGGAGTCCCG
CTGCGAAGATGCTCAAAGTCACGGTGCCCTCCTGCTCCGCCTCGTCCTGCTCTTCGG
TCACCGCCAGTGCGGCCCCGGGGACCGCGAGCCTCGTCCCGGATTACTGGATCGAC
GGCTCCAACAGGGATGCGCTGAGCGATTTCTTCGAGGTGGAGTCGGAGCTGGGACG
GGGTGCTACATCCATTGTGTACAGATGCAAACAGAAGGGGACCCAGAAGCCTTATG
CTCTCAAAGTGTTAAAGAAAACAGTGGACAAAAAAATCGTAAGAACTGAGATAGGA
GTTCTTCTTCGCCTCTCACATCCAAACATTATAAAACTTAAAGAGATATTTGAAACC
CCTACAGAAATCAGTCTGGTCCTAGAACTCGTCACAGGAGGAGAACTGTTTGATAG
GATTGTGGAAAAGGGATATTACAGTGAGCGAGATGCTGCAGATGCCGTTAAACAAA
TCCTGGAGGCAGTTGCTTATCTACATGAAAATGGGATTGTCCATCGTGATCTCAAAC
CAGAGAATCTTCTTTATGCAACTCCAGCCCCAGATGCACCACTCAAAATCGCTGATT
TTGGACTCTCTAAAATTGTGGAACATCAAGTGCTCATGAAGACAGTATGTGGAACCC
CAGGGTACTGCGCACCTGAAATTCTTAGAGGTTGTGCCTATGGACCTGAGGTGGACA
TGTGGTCTGTAGGAATAATCACCTACATCTTACTTTGTGGATTTGAACCATTCTATGA
TGAAAGAGGCGATCAGTTCATGTTCAGGAGAATTCTGAATTGTGAATATTACTTTAT
CTCCCCCTGGTGGGATGAAGTATCTCTAAATGCCAAGGACTTGGTCAGAAAATTAAT
TGTTTTGGATCCAAAGAAACGGCTGACTACATTTCAAGCTCTCCAGCATCCGTGGGT
CACAGGTAAAGCAGCCAATTTTGTACACATGGATACCGCTCAAAAGAAGCTCCAAG
AATTCAATGCCCGGCGTAAGCTTAAGGCAGCGGTGAAGGCTGTGGTGGCCTCTTCCC
GCCTGGGAAGTGCCAGCAGCAGCCATGGCAGCATCCAGGAGAGCCACAAGGCTAGC
CGAGACCCTTCTCCAATCCAAGATGGCAACGAGGACATGAAAGCTATTCCAGAAGG
AGAGAAAATTCAAGGCGATGGGGCCCAAGCCGCAGTTAAGGGGGCACAGGCTGAG
CTGATGAAGGTGCAAGCCTTAGAGAAAGTTAAAGGTGCAGATATAAATGCTGAAGA
GGCCCCCAAAATGGTGCCCAAGGCAGTGGAGGATGGGATAAAGGTGGCTGACCTGG
AACTAGAGGAGGGCCTAGCAGAGGAGAAGCTGAAGACTGTGGAGGAGGCAGCAGC
TCCCAGAGAAGGGCAAGGAAGCTCTGCTGTGGGTTTTGAAGTTCCACAGCAAGATG
TGATCCTGCCAGAGTACTAAACAGCTTCCTTCAGATCTGGAAGCCAAACACCGGCAT
TTTATGTACTTTGTCCTTCAGCAAGAAAGGTGTGGAAGCATGATATGTACTATAGTG
ATTCTGTTTTTGAGGTGCAAAAAACATACATATATACCAGTTGGTAATTCTAACTTC
AATGCATGTGACTGCTTTATGAAAATAATAGTGTCTTCTATGGCATGTAATGGATAC
CTAATACCGATGAGTTAAATCTTGCAAGTTAACACAACGTAACACTTAAAAGCATAC
ATTTTCAGCAACCAGTGGCACATATTTGAAGTGAATAGTAGCAAATTGTTTTTGCTTT
GAAAATCTAGCCATCCTACATCCTTTGGATTTCTTCACAAGGCAGTAATTCCTTTGAA
CTACTGCTTAGCTAATACTAGGTAGTGCTAAAAGACATGTTCCCATAACTTTTACAA
CATTTTACTTTTTATCATTGATGTGTTCAAACTGTTTACAAGGAGATGCTTATAGATG
ATAGTTGTACATATGTGCAAAAAAAAATCCACTTGCAATGGTAAGAAATTGAAGTA
TCCTTAAAGGCCATGAAGCCATATGTCCCTAAAAAAAAAAAAAAAAAAAAAAAAA
AAAAAAAA
CDKN1A (SEQ ID NO: 10; NM_000389.4).
GTTGTATATCAGGGCCGCGCTGAGCTGCGCCAGCTGAGGTGTGAGCAGCTGCCGAA
GTCAGTTCCTTGTGGAGCCGGAGCTGGGCGCGGATTCGCCGAGGCACCGAGGCACT
CAGAGGAGGCGCCATGTCAGAACCGGCTGGGGATGTCCGTCAGAACCCATGCGGCA
GCAAGGCCTGCCGCCGCCTCTTCGGCCCAGTGGACAGCGAGCAGCTGAGCCGCGAC
TGTGATGCGCTAATGGCGGGCTGCATCCAGGAGGCCCGTGAGCGATGGAACTTCGA
CTTTGTCACCGAGACACCACTGGAGGGTGACTTCGCCTGGGAGCGTGTGCGGGGCCT
TGGCCTGCCCAAGCTCTACCTTCCCACGGGGCCCCGGCGAGGCCGGGATGAGTTGG
GAGGAGGCAGGCGGCCTGGCACCTCACCTGCTCTGCTGCAGGGGACAGCAGAGGAA
GACCATGTGGACCTGTCACTGTCTTGTACCCTTGTGCCTCGCTCAGGGGAGCAGGCT
GAAGGGTCCCCAGGTGGACCTGGAGACTCTCAGGGTCGAAAACGGCGGCAGACCAG
CATGACAGATTTCTACCACTCCAAACGCCGGCTGATCTTCTCCAAGAGGAAGCCCTA
ATCCGCCCACAGGAAGCCTGCAGTCCTGGAAGCGCGAGGGCCTCAAAGGCCCGCTC
TACATCTTCTGCCTTAGTCTCAGTTTGTGTGTCTTAATTATTATTTGTGTTTTAATTTA
AACACCTCCTCATGTACATACCCTGGCCGCCCCCTGCCCCCCAGCCTCTGGCATTAG
AATTATTTAAACAAAAACTAGGCGGTTGAATGAGAGGTTCCTAAGAGTGCTGGGCA
TTTTTATTTTATGAAATACTATTTAAAGCCTCCTCATCCCGTGTTCTCCTTTTCCTCTC
TCCCGGAGGTTGGGTGGGCCGGCTTCATGCCAGCTACTTCCTCCTCCCCACTTGTCC
GCTGGGTGGTACCCTCTGGAGGGGTGTGGCTCCTTCCCATCGCTGTCACAGGCGGTT
ATGAAATTCACCCCCTTTCCTGGACACTCAGACCTGAATTCTTTTTCATTTGAGAAGT
AAACAGATGGCACTTTGAAGGGGCCTCACCGAGTGGGGGCATCATCAAAAACTTTG
GAGTCCCCTCACCTCCTCTAAGGTTGGGCAGGGTGACCCTGAAGTGAGCACAGCCTA
GGGCTGAGCTGGGGACCTGGTACCCTCCTGGCTCTTGATACCCCCCTCTGTCTTGTG
AAGGCAGGGGGAAGGTGGGGTCCTGGAGCAGACCACCCCGCCTGCCCTCATGGCCC
CTCTGACCTGCACTGGGGAGCCCGTCTCAGTGTTGAGCCTTTTCCCTCTTTGGCTCCC
CTGTACCTTTTGAGGAGCCCCAGCTACCCTTCTTCTCCAGCTGGGCTCTGCAATTCCC
CTCTGCTGCTGTCCCTCCCCCTTGTCCTTTCCCTTCAGTACCCTCTCAGCTCCAGGTG
GCTCTGAGGTGCCTGTCCCACCCCCACCCCCAGCTCAATGGACTGGAAGGGGAAGG
GACACACAAGAAGAAGGGCACCCTAGTTCTACCTCAGGCAGCTCAAGCAGCGACCG
CCCCCTCCTCTAGCTGTGGGGGTGAGGGTCCCATGTGGTGGCACAGGCCCCCTTGAG
TGGGGTTATCTCTGTGTTAGGGGTATATGATGGGGGAGTAGATCTTTCTAGGAGGGA
GACACTGGCCCCTCAAATCGTCCAGCGACCTTCCTCATCCACCCCATCCCTCCCCAG
TTCATTGCACTTTGATTAGCAGCGGAACAAGGAGTCAGACATTTTAAGATGGTGGCA
GTAGAGGCTATGGACAGGGCATGCCACGTGGGCTCATATGGGGCTGGGAGTAGTTG
TCTTTCCTGGCACTAACGTTGAGCCCCTGGAGGCACTGAAGTGCTTAGTGTACTTGG
AGTATTGGGGTCTGACCCCAAACACCTTCCAGCTCCTGTAACATACTGGCCTGGACT
GTTTTCTCTCGGCTCCCCATGTGTCCTGGTTCCCGTTTCTCCACCTAGACTGTAAACC
TCTCGAGGGCAGGGACCACACCCTGTACTGTTCTGTGTCTTTCACAGCTCCTCCCAC
AATGCTGAATATACAGCAGGTGCTCAATAAATGATTCTTAGTGACTTTACTTGTAAA
AAAAAAAAAAAAAAA
COCH (SEQ ID NO: 11; AY358900.1).
GGGGCCTTGCCTTCCGCACTCGGGCGCAGCCGGGTGGATCTCGAGCAGGTGCGGAG
CCCCGGGCGGCGGGCGCGGGTGCGAGGGATCCCTGACGCCTCTGTCCCTGTTTCTTT
GTCGCTCCCAGCCTGTCTGTCGTCGTTTTGGCGCCCCCGCCTCCCCGCGGTGCGGGG
TTGCACACCGATCCTGGGCTTCGCTCGATTTGCCGCCGAGGCGCCTCCCAGACCTAG
AGGGGCGCTGGCCTGGAGCAGCGGGTCGTCTGTGTCCTCTCTCCTCTGCGCCGCGCC
CGGGGATCCGAAGGGTGCGGGGCTCTGAGGAGGTGACGCGCGGGGCCTCCCGCACC
CTGGCCTTGCCCGCATTCTCCCTCTCTCCCAGGTGTGAGCAGCCTATCAGTCACCATG
TCCGCAGCCTGGATCCCGGCTCTCGGCCTCGGTGTGTGTCTGCTGCTGCTGCCGGGG
CCCGCGGGCAGCGAGGGAGCCGCTCCCATTGCTATCACATGTTTTACCAGAGGCTTG
GACATCAGGAAAGAGAAAGCAGATGTCCTCTGCCCAGGGGGCTGCCCTCTTGAGGA
ATTCTCTGTGTATGGGAACATAGTATATGCTTCTGTATCGAGCATATGTGGGGCTGC
TGTCCACAGGGGAGTAATCAGCAACTCAGGGGGACCTGTACGAGTCTATAGCCTAC
CTGGTCGAGAAAACTATTCCTCAGTAGATGCCAATGGCATCCAGTCTCAAATGCTTT
CTAGATGGTCTGCTTCTTTCACAGTAACTAAAGGCAAAAGTAGTACACAGGAGGCC
ACAGGACAAGCAGTGTCCACAGCACATCCACCAACAGGTAAACGACTAAAGAAAA
CACCCGAGAAGAAAACTGGCAATAAAGATTGTAAAGCAGACATTGCATTTCTGATT
GATGGAAGCTTTAATATTGGGCAGCGCCGATTTAATTTACAGAAGAATTTTGTTGGA
AAAGTGGCTCTAATGTTGGGAATTGGAACAGAAGGACCACATGTGGGCCTTGTTCA
AGCCAGTGAACATCCCAAAATAGAATTTTACTTGAAAAACTTTACATCAGCCAAAG
ATGTTTTGTTTGCCATAAAGGAAGTAGGTTTCAGAGGGGGTAATTCCAATACAGGAA
AAGCCTTGAAGCATACTGCTCAGAAATTCTTCACGGTAGATGCTGGAGTAAGAAAA
GGGATCCCCAAAGTGGTGGTGGTATTTATTGATGGTTGGCCTTCTGATGACATCGAG
GAAGCAGGCATTGTGGCCAGAGAGTTTGGTGTCAATGTATTTATAGTTTCTGTGGCC
AAGCCTATCCCTGAAGAACTGGGGATGGTTCAGGATGTCACATTTGTTGACAAGGCT
GTCTGTCGGAATAATGGCTTCTTCTCTTACCACATGCCCAACTGGTTTGGCACCACA
AAATACGTAAAGCCTCTGGTACAGAAGCTGTGCACTCATGAACAAATGATGTGCAG
CAAGACCTGTTATAACTCAGTGAACATTGCCTTTCTAATTGATGGCTCCAGCAGTGT
TGGAGATAGCAATTTCCGCCTCATGCTTGAATTTGTTTCCAACATAGCCAAGACTTTT
GAAATCTCGGACATTGGTGCCAAGATAGCTGCTGTACAGTTTACTTATGATCAGCGC
ACGGAGTTCAGTTTCACTGACTATAGCACCAAAGAGAATGTCCTAGCTGTCATCAGA
AACATCCGCTATATGAGTGGTGGAACAGCTACTGGTGATGCCATTTCCTTCACTGTT
AGAAATGTGTTTGGCCCTATAAGGGAGAGCCCCAACAAGAACTTCCTAGTAATTGTC
ACAGATGGGCAGTCCTATGATGATGTCCAAGGCCCTGCAGCTGCTGCACATGATGCA
GGAATCACTATCTTCTCTGTTGGTGTGGCTTGGGCACCTCTGGATGACCTGAAAGAT
ATGGCTTCTAAACCGAAGGAGTCTCACGCTTTCTTCACAAGAGAGTTCACAGGATTA
GAACCAATTGTTTCTGATGTCATCAGAGGCATTTGTAGAGATTTCTTAGAATCCCAG
CAATAATGGTAACATTTTGACAACTGAAAGAAAAAGTACAAGGGGATCCAGTGTGT
AAATTGTATTCTCATAATACTGAAATGCTTTAGCATACTAGAATCAGATACAAAACT
ATTAAGTATGTCAACAGCCATTTAGGCAAATAAGCACTCCTTTAAAGCCGCTGCCTT
CTGGTTACAATTTACAGTGTACTTTGTTAAAAACACTGCTGAGGCTTCATAATCATG
GCTCTTAGAAACTCAGGAAAGAGGAGATAATGTGGATTAAAACCTTAAGAGTTCTA
ACCATGCCTACTAAATGTACAGATATGCAAATTCCATAGCTCAATAAAAGAATCTGA
TACTTAGACCAAAAAAAAAAA
DHRS4 (SEQ ID NO: 12; NM_021004.3).
CTACTCTGTCACCGCCCCTGGGAAGAGTGGAACCCATACTTGCTGGTCTGATCCATG
CACAAGGCGGGGCTGCTAGGCCTCTGTGCCCGGGCTTGGAATTCGGTGCGGATGGC
CAGCTCCGGGATGACCCGCCGGGACCCGCTCGCAAATAAGGTGGCCCTGGTAACGG
CCTCCACCGACGGGATCGGCTTCGCCATCGCCCGGCGTTTGGCCCAGGACGGGGCCC
ATGTGGTCGTCAGCAGCCGGAAGCAGCAGAATGTGGACCAGGCGGTGGCCACGCTG
CAGGGGGAGGGGCTGAGCGTGACGGGCACCGTGTGCCATGTGGGGAAGGCGGAGG
ACCGGGAGCGGCTGGTGGCCACGGCTGTGAAGCTTCATGGAGGTATCGATATCCTA
GTCTCCAATGCTGCTGTCAACCCTTTCTTTGGAAGCATAATGGATGTCACTGAGGAG
GTGTGGGACAAGACTCTGGACATTAATGTGAAGGCCCCAGCCCTGATGACAAAGGC
AGTGGTGCCAGAAATGGAGAAACGAGGAGGCGGCTCAGTGGTGATCGTGTCTTCCA
TAGCAGCCTTCAGTCCATCTCCTGGCTTCAGTCCTTACAATGTCAGTAAAACAGCCTT
GCTGGGCCTGACCAAGACCCTGGCCATAGAGCTGGCCCCAAGGAACATTAGGGTGA
ACTGCCTAGCACCTGGACTTATCAAGACTAGCTTCAGCAGGATGCTCTGGATGGACA
AGGAAAAAGAGGAAAGCATGAAAGAAACCCTGCGGATAAGAAGGTTAGGCGAGCC
AGAGGATTGTGCTGGCATCGTGTCTTTCCTGTGCTCTGAAGATGCCAGCTACATCAC
TGGGGAAACAGTGGTGGTGGGTGGAGGAACCCCGTCCCGCCTCTGAGGACCGGGAG
ACAGCCCACAGGCCAGAGTTGGGCTCTAGCTCCTGGTGCTGTTCCCGCATTCACCCA
CTGGCCTTTCCCACCTCTGCTCACCTTACTGTTCACCTCATCAAATCAGTTCTGCCCT
GTGAAAAGATCCAGCCTTCCCTGCCGTCAAGGTGGCGTCTTACTCGGGATTTCTGCT
GTTGTTGTGGCCTTGGGTAAAGGCCTCCCCTGAGAACACAGGACAGGCCTGCTGACA
AGGCTGAGTCTACCTTGGCAAAGACCAAGATATTTTTTCCCGGGCCACTGGGGAATC
TGAGGGGTGATGGGAGAGAAGGAACCTGGAGTGGAAGGAGCAGAGTTGCAAATTA
ACAACTTGCAAATGAGGTGCAAATAAAATGCAGATGATTGCGCGGCTTTGAATCCA
AAAAAAAAAAAAAAAAA
MICAL1 (SEQ ID NO: 13; NM_022765.3).
CCCAAGACTGTCCCCGCTGGAGGCGGTAGAGGGATCCAGAAGTAATGAGATGCTAA
TGAGTCGCGAATAAAGCCCGGGCGGCGCCCCGCGCCCCTCGCGGAAGCCCACACTC
CGCGCGACTCCAGGCGCACGCCCCGGGCCGCCCCGCATCCCAGCATCCCCGCCCGA
TCTCGGCGTTTCCGCCCCCGCCCCCGCCCCCGCCCTCCCACCCGCTCAGACCTGGTTG
CCAGCCCAACAGGAAGCGGCCCCTCCCGGCTTCGGAGCCGCCGCCACTCATCTCTGC
CCAGCTGCTGCCCTCCCCAGGAGGCCTCCATGGCTTCACCTACCTCCACCAACCCAG
CGCATGCCCACTTTGAGAGCTTCCTGCAGGCCCAGCTGTGCCAGGACGTGCTGAGCA
GCTTCCAGGAGCTGTGTGGGGCCCTGGGGCTGGAACCCGGTGGGGGGCTGCCCCAG
TACCACAAGATCAAGGACCAGCTCAACTACTGGAGCGCCAAGTCACTGTGGACCAA
GCTGGACAAGCGAGCAGGCCAGCCTGTCTACCAGCAGGGCCGGGCCTGCACCAGCA
CCAAGTGCCTGGTGGTGGGTGCTGGACCTTGCGGGCTGCGGGTCGCTGTGGAGCTGG
CGCTGCTGGGGGCCCGAGTGGTGCTGGTGGAAAAGCGCACCAAGTTCTCTCGCCAC
AACGTGCTCCACCTCTGGCCCTTCACCATCCACGACCTGCGGGCACTCGGTGCTAAG
AAGTTCTACGGGCGCTTCTGCACCGGCACCCTGGACCACATCAGCATCAGGCAGCTC
CAGCTGCTTCTGCTGAAGGTAGCATTGCTGCTGGGGGTGGAAATTCACTGGGGTGTC
ACTTTCACTGGCCTCCAGCCCCCTCCTAGGAAGGGGAGTGGCTGGCGTGCCCAGCTC
CAACCCAACCCCCCTGCCCAGCTGGCCAACTATGAATTTGACGTCCTTATCTCGGCT
GCAGGAGGTAAATTCGTCCCTGAAGGCTTCAAAGTTCGAGAAATGCGAGGCAAACT
GGCCATTGGCATCACAGCCAACTTTGTGAATGGACGCACCGTGGAGGAGACACAGG
TGCCGGAGATCAGTGGTGTAGCCAGGATCTACAACCAGAGCTTCTTCCAGAGCCTTC
TCAAAGCCACAGGCATTGATCTGGAGAACATTGTGTACTACAAGGACGACACCCAC
TACTTTGTGATGACAGCCAAGAAGCAGTGCCTGCTGCGGCTGGGGGTGCTGCGCCA
GGACTGGCCAGACACCAATCGGCTGCTGGGCAGTGCCAATGTGGTGCCCGAGGCTC
TGCAGCGCTTTACCCGGGCAGCTGCTGACTTTGCCACCCATGGCAAGCTCGGGAAAC
TAGAGTTTGCCCAGGATGCCCATGGGCAGCCTGATGTCTCTGCCTTTGACTTCACGA
GCATGATGCGGGCAGAGAGTTCTGCTCGTGTGCAAGAGAAGCATGGCGCCCGCCTG
CTGCTGGGACTGGTGGGGGACTGCCTGGTGGAGCCCTTCTGGCCCCTGGGCACTGGA
GTGGCACGGGGCTTCCTGGCAGCCTTTGATGCAGCCTGGATGGTGAAGCGGTGGGC
AGAGGGCGCTGAGTCCCTAGAGGTGTTGGCTGAGCGTGAGAGCCTGTACCAGCTTCT
GTCACAGACATCCCCAGAAAACATGCATCGCAATGTGGCCCAGTATGGGCTGGACC
CAGCCACCCGCTACCCCAACCTGAACCTCCGGGCAGTGACCCCCAATCAGGTACGA
GACCTGTATGATGTGCTAGCCAAGGAGCCTGTGCAGAGGAACAACGACAAGACAGA
TACAGGGATGCCAGCCACCGGGTCGGCAGGCACCCAGGAGGAGCTGCTACGCTGGT
GCCAGGAGCAGACAGCTGGGTACCCGGGAGTCCACGTCTCCGATTTGTCTTCCTCCT
GGGCTGATGGGCTAGCTCTGTGTGCCCTGGTGTACCGGCTGCAGCCTGGCCTGCTGG
AACCCTCAGAGCTGCAGGGGCTGGGAGCTCTGGAAGCAACTGCTTGGGCACTAAAG
GTGGCAGAGAATGAGCTGGGCATCACACCGGTGGTGTCTGCACAGGCCGTGGTAGC
AGGGAGTGACCCACTGGGCCTCATTGCCTACCTCAGCCACTTCCACAGTGCCTTCAA
GAGCATGGCCCACAGCCCAGGCCCTGTCAGCCAGGCCTCCCCAGGGACCTCCAGTG
CTGTATTATTCCTTAGTAAACTTCAGAGGACCCTGCAGCGATCCCGGGCCAAGGAAA
ATGCAGAGGATGCTGGTGGCAAGAAGCTGCGCTTGGAGATGGAGGCCGAGACCCCA
AGTACTGAGGTGCCACCTGACCCAGAGCCTGGTGTACCCCTGACACCCCCATCCCAA
CACCAGGAGGCCGGTGCTGGGGACCTGTGTGCACTTTGTGGGGAACACCTCTATGTC
CTGGAACGCCTCTGTGTCAACGGCCATTTCTTCCACCGGAGCTGCTTCCGCTGCCAT
ACCTGTGAGGCCACACTGTGGCCAGGTGGCTACGAGCAGCACCCAGGAGATGGACA
TTTCTACTGCCTCCAGCACCTGCCCCAGACAGACCACAAAGCGGAAGGCAGCGATA
GAGGCCCTGAGAGTCCGGAGCTCCCCACACCAAGTGAGAATAGCATGCCACCAGGC
CTCTCAACTCCCACAGCCTCGCAGGAGGGGGCCGGTCCTGTTCCAGATCCCAGCCAG
CCCACCCGTCGGCAGATCCGCCTCTCCAGCCCGGAGCGCCAGCGGTTGTCCTCCCTT
AACCTTACCCCTGACCCGGAAATGGAGCCTCCACCCAAGCCTCCCCGCAGCTGCTCC
GCCTTGGCCCGCCACGCCCTGGAGAGCAGCTTTGTGGGCTGGGGCCTGCCAGTCCAG
AGCCCTCAAGCTCTTGTGGCCATGGAGAAGGAGGAAAAAGAGAGTCCCTTCTCCAG
TGAAGAGGAAGAAGAAGATGTGCCTTTGGACTCAGATGTGGAACAGGCCCTGCAGA
CCTTTGCCAAGACCTCAGGCACCATGAATAACTACCCAACATGGCGTCGGACTCTGC
TGCGCCGTGCGAAGGAGGAGGAGATGAAGAGGTTCTGCAAGGCCCAGACCATCCAA
CGGCGACTAAATGAGATTGAGGCTGCCTTGAGGGAGCTAGAGGCCGAGGGCGTGAA
GCTGGAGCTGGCCTTGAGGCGCCAGAGCAGTTCCCCAGAACAGCAAAAGAAACTAT
GGGTAGGACAGCTGCTACAGCTCGTTGACAAGAAAAACAGCCTGGTGGCTGAGGAG
GCCGAGCTCATGATCACGGTGCAGGAATTGAATCTGGAGGAGAAACAGTGGCAGCT
GGACCAGGAGCTACGAGGCTACATGAACCGGGAAGAAAACCTAAAGACAGCTGCT
GATCGGCAGGCTGAGGACCAGGTCCTGAGGAAGCTGGTGGATTTGGTCAACCAGAG
AGATGCCCTCATCCGCTTCCAGGAGGAGCGCAGGCTCAGCGAGCTGGCCTTGGGGA
CAGGGGCCCAGGGCTAGACGAGGGTGGGCCGTCTGCTTTCGTTCCCACAAAGAAAG
CACCTCACCCCAGCACAGTGCCACCCCTGTTCATCTGGGCTGCCTGGCAGAGAGCCT
TGCTGTTTACAATTAAAATGTTTCTGCCACAAAAAAAAAAAAAAAAAAA
MOB3B (SEQ ID NO: 14; AJ580636.1).
ATGTCCATAGCCCTGAAGCAGGTATTCAACAAGGACAAGACCTTCCGACCCAAGAG
GAAATTTGAACCTGGCACACAGAGGTTTGAGCTGCACAAACGGGCTCAGGCATCCC
TCAACTCGGGTGTGGACCTGAAGGCGGCTGTGCAGTTGCCCAGTGGGGAGGACCAG
AATGACTGGGTGGCAGTACATGTGGTGGACTTCTTCAATCGGATCAACCTCATCTAT
GGCACCATCTGTGAGTTCTGCACCGAGCGGACCTGTCCTGTGATGTCAGGGGGCCCC
AAATATGAGTATCGGTGGCAGGATGATCTCAAGTATAAGAAGCCAACAGCGCTGCC
AGCTCCCCAGTACATGAACCTTCTTATGGATTGGATTGAGGTTCAGATCAACAACGA
GGAAATATTTCCAACATGCGTGGGTGTTCCCTTCCCAAAGAACTTCCTTCAGATCTG
CAAGAAGATCCTGTGCCGCCTTTTCCGGGTCTTTGTCCACGTCTATATCCACCACTTC
GACCGGGTCATTGTGATGGGTGCAGAGGCCCATGTCAACACCTGCTACAAACACTTC
TATTACTTTGTCACAGAGATGAACCTCATAGACCGCAAGGAGCTAGAGCCTTTGAAA
GAAATGACGAGCAGGATGTGTCACTAA
NUSAP1 (SEQ ID NO: 15; NM_016359.4).
GCGTTACAGGCCCTTTGGCGCCTGCGTATTCGTGAAGTGTGAAAAAAGCGCGCCTCT
GTTGGGACGGGAAATCAGCCTTTCTATTGGTCAGGGTTAGAAACCCCGCCTTTGAGG
CATTTTCAACCAATGGAAGCGCGGCATTCTTCATTTAAACTGTCTATAAATTTCTGCC
TAGTCAAAGTTAAGAGTGGCGCCAGGGATTTGAACCGCGCTGACGAAGTTTGGTGA
TCCATCTTCCGAGTATCGCCGGGATTTCGAATCGCGATGATCATCCCCTCTCTAGAG
GAGCTGGACTCCCTCAAGTACAGTGACCTGCAGAACTTAGCCAAGAGTCTGGGTCTC
CGGGCCAACCTGAGGGCAACCAAGTTGTTAAAAGCCTTGAAAGGCTACATTAAACA
TGAGGCAAGAAAAGGAAATGAGAATCAGGATGAAAGTCAAACTTCTGCATCCTCTT
GTGATGAGACTGAGATACAGATCAGCAACCAGGAAGAAGCTGAGAGACAGCCACTT
GGCCATGTCACCAAAACAAGGAGAAGGTGCAAGACTGTCCGTGTGGACCCTGACTC
ACAGCAGAATCATTCAGAGATAAAAATAAGTAATCCCACTGAATTCCAGAATCATG
AAAAGCAGGAAAGCCAGGATCTCAGAGCTACTGCAAAAGTTCCTTCTCCACCAGAC
GAGCACCAAGAAGCTGAGAATGCTGTTTCCTCAGGTAACAGAGATTCAAAGGTACC
TTCAGAAGGAAAGAAATCTCTCTACACAGATGAGTCATCCAAACCTGGAAAAAATA
AAAGAACTGCAATCACTACTCCAAACTTTAAGAAGCTTCATGAAGCTCATTTTAAGG
AAATGGAGTCCATTGATCAATATATTGAGAGAAAAAAGAAACATTTTGAAGAACAC
AATTCCATGAATGAACTGAAGCAGCAGCCCATCAATAAGGGAGGGGTCAGGACTCC
AGTACCTCCAAGAGGAAGACTCTCTGTGGCTTCTACTCCCATCAGCCAACGACGCTC
GCAAGGCCGGTCTTGTGGCCCTGCAAGTCAGAGTACCTTGGGTCTGAAGGGGTCACT
CAAGCGCTCTGCTATCTCTGCAGCTAAAACGGGTGTCAGGTTTTCAGCTGCTACTAA
AGATAATGAGCATAAGCGTTCACTGACCAAGACTCCAGCCAGAAAGTCTGCACATG
TGACCGTGTCTGGGGGCACCCCAAAAGGCGAGGCTGTGCTTGGGACACACAAATTA
AAGACCATCACGGGGAATTCTGCTGCTGTTATTACCCCATTCAAGTTGACAACTGAG
GCAACGCAGACTCCAGTCTCCAATAAGAAACCAGTGTTTGATCTTAAAGCAAGTTTG
TCTCGTCCCCTCAACTATGAACCACACAAAGGAAAGCTAAAACCATGGGGGCAATC
TAAAGAAAATAATTATCTAAATCAACATGTCAACAGAATTAACTTCTACAAGAAAA
CTTACAAACAACCCCATCTCCAGACAAAGGAAGAGCAACGGAAGAAACGCGAGCA
AGAACGAAAGGAGAAGAAAGCAAAGGTTTTGGGAATGCGAAGGGGCCTCATTTTGG
CTGAAGATTAATAATTTTTTAACATCTTGTAAATATTCCTGTATTCTCAACTTTTTTCC
TTTTGTAAATTTTTTTTTTTTGCTGTCATCCCCACTTTAGTCACGAGATCTTTTTCTGC
TAACTGTTCATAGTCTGTGTAGTGTCCATGGGTTCTTCATGTGCTATGATCTCTGAAA
AGACGTTATCACCTTAAAGCTCAAATTCTTTGGGATGGTTTTTACTTAAGTCCATTAA
CAATTCAGGTTTCTAACGAGACCCATCCTAAAATTCTGTTTCTAGATTTTTAATGTCA
AGTTCCCAAGTTCCCCCTGCTGGTTCTAATATTAACAGAACTGCAGTCTTCTGCTAGC
CAATAGCATTTACCTGATGGCAGCTAGTTATGCAAGCTTCAGGAGAATTTGAACAAT
AACAAGAATAGGGTAAGCTGGGATAGAAAGGCCACCTCTTCACTCTCTATAGAATA
TAGTAACCTTTATGAAACGGGGCCATATAGTTTGGTTATGACATCAATATTTTACCT
AGGTGAAATTGTTTAGGCTTATGTACCTTCGTTCAAATATCCTCATGTAATTGCCATC
TGTCACTCACTATATTCACAAAAATAAAACTCTACAACTCATTCTAACATTGCTTACT
TAAAAGCTACATAGCCCTATCGAAATGCGAGGATTAATGCTTTAATGCTTTTAGAGA
CAGGGTCTCACTGTGTTGCCCAGGCTGGTCTCAAACTCCACCAAATGTACTTCTTATT
CATTTTATGGAAAAGACTAGGCTTTGCTTAGTATCATGTCCATGTTTCCTTCACCTCA
GTGGAGCTTCTGAGTTTTATACTGCTCAAGATCGTCATAAATAAAATTTTTTCTCATT
GTCATAGAAAAAAAAAAAAAAAAAA
IL27RA (SEQ ID NO: 16; NM_004843.3).
GCGGAGGCGGCCTGCCGGGGTGGTTCGGCTTCCCGTTGCCGCCTCGGGCGCTGTACC
CAGAGCTCGAAGAGGAGCAGCGCGGCCGCGCGGACCCGGCAAGGCTGGGCCGGAC
TCGGGGCTCCCGAGGGACGCCATGCGGGGAGGCAGGGGCGCCCCTTTCTGGCTGTG
GCCGCTGCCCAAGCTGGCGCTGCTGCCTCTGTTGTGGGTGCTTTTCCAGCGGACGCG
TCCCCAGGGCAGCGCCGGGCCACTGCAGTGCTACGGAGTTGGACCCTTGGGCGACT
TGAACTGCTCGTGGGAGCCTCTTGGGGACCTGGGAGCCCCCTCCGAGTTACACCTCC
AGAGCCAAAAGTACCGTTCCAACAAAACCCAGACTGTGGCAGTGGCAGCCGGACGG
AGCTGGGTGGCCATTCCTCGGGAACAGCTCACCATGTCTGACAAACTCCTTGTCTGG
GGCACTAAGGCAGGCCAGCCTCTCTGGCCCCCCGTCTTCGTGAACCTAGAAACCCAA
ATGAAGCCAAACGCCCCCCGGCTGGGCCCTGACGTGGACTTTTCCGAGGATGACCC
CCTGGAGGCCACTGTCCATTGGGCCCCACCTACATGGCCATCTCATAAAGTTCTGAT
CTGCCAGTTCCACTACCGAAGATGTCAGGAGGCGGCCTGGACCCTGCTGGAACCGG
AGCTGAAGACCATACCCCTGACCCCTGTTGAGATCCAAGATTTGGAGCTAGCCACTG
GCTACAAAGTGTATGGCCGCTGCCGGATGGAGAAAGAAGAGGATTTGTGGGGCGAG
TGGAGCCCCATTTTGTCCTTCCAGACACCGCCTTCTGCTCCAAAAGATGTGTGGGTA
TCAGGGAACCTCTGTGGGACGCCTGGAGGAGAGGAACCTTTGCTTCTATGGAAGGC
CCCAGGGCCCTGTGTGCAGGTGAGCTACAAAGTCTGGTTCTGGGTTGGAGGTCGTGA
GCTGAGTCCAGAAGGAATTACCTGCTGCTGCTCCCTAATTCCCAGTGGGGCGGAGTG
GGCCAGGGTGTCCGCTGTCAACGCCACAAGCTGGGAGCCTCTCACCAACCTCTCTTT
GGTCTGCTTGGATTCAGCCTCTGCCCCCCGTAGCGTGGCAGTCAGCAGCATCGCTGG
GAGCACGGAGCTACTGGTGACCTGGCAACCGGGGCCTGGGGAACCACTGGAGCATG
TAGTGGACTGGGCTCGAGATGGGGACCCCCTGGAGAAACTCAACTGGGTCCGGCTT
CCCCCTGGGAACCTCAGTGCTCTGTTACCAGGGAATTTCACTGTCGGGGTCCCCTAT
CGAATCACTGTGACCGCAGTCTCTGCTTCAGGCTTGGCCTCTGCATCCTCCGTCTGG
GGGTTCAGGGAGGAATTAGCACCCCTAGTGGGGCCAACGCTTTGGCGACTCCAAGA
TGCCCCTCCAGGGACCCCCGCCATAGCGTGGGGAGAGGTCCCAAGGCACCAGCTTC
GAGGCCACCTCACCCACTACACCTTGTGTGCACAGAGTGGAACCAGCCCCTCCGTCT
GCATGAATGTGAGTGGCAACACACAGAGTGTCACCCTGCCTGACCTTCCTTGGGGTC
CCTGTGAGCTGTGGGTGACAGCATCTACCATCGCTGGACAGGGCCCTCCTGGTCCCA
TCCTCCGGCTTCATCTACCAGATAACACCCTGAGGTGGAAAGTTCTGCCGGGCATCC
TATTCTTGTGGGGCTTGTTCCTGTTGGGGTGTGGCCTGAGCCTGGCCACCTCTGGAA
GGTGCTACCACCTAAGGCACAAAGTGCTGCCCCGCTGGGTCTGGGAGAAAGTTCCT
GATCCTGCCAACAGCAGTTCAGGCCAGCCCCACATGGAGCAAGTACCTGAGGCCCA
GCCCCTTGGGGACTTGCCCATCCTGGAAGTGGAGGAGATGGAGCCCCCGCCGGTTA
TGGAGTCCTCCCAGCCCGCCCAGGCCACCGCCCCGCTTGACTCTGGGTATGAGAAGC
ACTTCCTGCCCACACCTGAGGAGCTGGGCCTTCTGGGGCCCCCCAGGCCACAGGTTC
TGGCCTGAACCACACGTCTGGCTGGGGGCTGCCAGCCAGGCTAGAGGGATGCTCAT
GCAGGTTGCACCCCAGTCCTGGATTAGCCCTCTTGATGGATGAAGACACTGAGGACT
CAGAGAGGCTGAGTCACTTACCTGAGGACACCCAGCCAGGCAGAGCTGGGATTGAA
GGACCCCTATAGAGAAGGGCTTGGCCCCCATGGGGAAGACACGGATGGAAGGTGGA
GCAAAGGAAAATACATGAAATTGAGAGTGGCAGCTGCCTGCCAAAATCTGTTCCGC
TGTAACAGAACTGAATTTGGACCCCAGCACAGTGGCTCACGCCTGTAATCCCAGCAC
TTTGGCAGGCCAAGGTGGAAGGATCACTTAGAGCTAGGAGTTTGAGACCAGCCTGG
GCAATATAGCAAGACCCCTCACTACAAAAATAAAACATCAAAAACAAAAACAATTA
GCTGGGCATGATGGCACACACCTGTAGTCCGAGCCACTTGGGAGGCTGAGGTGGGA
GGATCGGTTGAGCCCAGGAGTTCGAAGCTGCAGGGACCTCTGATTGCACCACTGCA
CTCCAGGCTGGGTAACAGAATGAGACCTTATCTCAAAAATAAACAAACTAATAAAA
AGCAAAAAAAAAAAAAAAAAAAAAAAAAAAA
HBA2 (SEQ ID NO: 17; NM_000517.4).
CATAAACCCTGGCGCGCTCGCGGGCCGGCACTCTTCTGGTCCCCACAGACTCAGAGA
GAACCCACCATGGTGCTGTCTCCTGCCGACAAGACCAACGTCAAGGCCGCCTGGGG
TAAGGTCGGCGCGCACGCTGGCGAGTATGGTGCGGAGGCCCTGGAGAGGATGTTCC
TGTCCTTCCCCACCACCAAGACCTACTTCCCGCACTTCGACCTGAGCCACGGCTCTG
CCCAGGTTAAGGGCCACGGCAAGAAGGTGGCCGACGCGCTGACCAACGCCGTGGCG
CACGTGGACGACATGCCCAACGCGCTGTCCGCCCTGAGCGACCTGCACGCGCACAA
GCTTCGGGTGGACCCGGTCAACTTCAAGCTCCTAAGCCACTGCCTGCTGGTGACCCT
GGCCGCCCACCTCCCCGCCGAGTTCACCCCTGCGGTGCACGCCTCCCTGGACAAGTT
CCTGGCTTCTGTGAGCACCGTGCTGACCTCCAAATACCGTTAAGCTGGAGCCTCGGT
AGCCGTTCCTCCTGCCCGCTGGGCCTCCCAACGGGCCCTCCTCCCCTCCTTGCACCG
GCCCTTCCTGGTCTTTGAATAAAGTCTGAGTGGGCAGCAAAAAAAAAAAAAAAAAA
PPM1F (SEQ ID NO: 18; NM_014634).
AGGGACGGGAAGTGGGCGGGGCCGGCCGGCAGCAGCTTGCGGGACACGGAGCCGC
GAGGAGACAGCTGAGGCCCGCGGAGACCAGGGGGTGAAGCCTGGAGACCCTCTTGC
CCTGGCCTAGCTGCAGGCCCCCGGGATGCTTTGGGCATGTCCTCTGGAGCCCCACAG
AAGAGCAGCCCAATGGCCAGTGGAGCTGAGGAGACCCCAGGCTTCCTGGACACGCT
CCTGCAAGACTTCCCAGCCCTGCTGAACCCAGAGGACCCTCTGCCATGGAAGGCCCC
AGGGACGGTGCTCAGCCAGGAGGAGGTGGAGGGCGAGCTGGCTGAGCTGGCCATG
GGCTTTCTGGGCAGCAGGAAGGCCCCGCCACCACTTGCTGCTGCTCTGGCCCACGAA
GCAGTTTCACAGCTGCTACAGACAGACCTTTCCGAATTCAGGAAGTTGCCCAGGGAG
GAAGAAGAAGAGGAGGAGGACGATGACGAGGAGGAAAAGGCCCCTGTGACCTTGC
TGGATGCCCAAAGCCTGGCACAGAGTTTCTTTAACCGCCTTTGGGAAGTCGCCGGCC
AGTGGCAGAAGCAGGTGCCATTGGCTGCCCGGGCCTCACAGCGGCAGTGGCTGGTC
TCCATCCACGCCATCCGGAACACTCGCCGCAAGATGGAGGACCGGCACGTGTCCCT
CCCTTCCTTCAACCAGCTCTTCGGCTTGTCTGACCCTGTGAACCGCGCCTACTTTGCT
GTGTTTGATGGTCACGGAGGCGTGGATGCTGCGAGGTACGCCGCTGTCCACGTGCAC
ACCAACGCTGCCCGCCAGCCAGAGCTGCCCACAGACCCTGAGGGAGCCCTCAGAGA
AGCCTTCCGGCGCACCGACCAGATGTTTCTCAGGAAAGCCAAGCGAGAGCGGCTGC
AGAGCGGCACCACAGGTGTGTGTGCGCTCATTGCAGGAGCGACCCTGCACGTCGCC
TGGCTCGGGGATTCCCAGGTCATTTTGGTACAGCAGGGACAGGTGGTGAAGCTGAT
GGAGCCACACAGACCAGAACGGCAGGATGAGAAGGCGCGCATTGAAGCATTGGGT
GGCTTTGTGTCTCACATGGACTGCTGGAGAGTCAACGGGACCCTGGCCGTCTCCAGA
GCCATCGGGGATGTCTTCCAGAAGCCCTACGTGTCTGGGGAGGCCGATGCAGCTTCC
CGGGCGCTGACGGGCTCCGAGGACTACCTGCTGCTTGCCTGTGATGGCTTCTTTGAC
GTCGTACCCCACCAGGAAGTTGTTGGCCTGGTCCAGAGCCACCTGACCAGGCAGCA
GGGCAGCGGGCTCCGTGTCGCCGAGGAGCTGGTGGCTGCGGCCCGGGAGCGGGGCT
CCCACGACAACATCACGGTCATGGTGGTCTTCCTCAGGGACCCCCAAGAGCTGCTGG
AGGGCGGGAACCAGGGAGAAGGGGACCCCCAGGCAGAAGGGAGGAGGCAGGACTT
GCCCTCCAGCCTTCCAGAACCTGAGACCCAGGCTCCACCAAGAAGCTAGGTGGTTTC
CAGGCCCCTGCCCTCCCCTTCCTCCCATCCTTGTCCTTCTCTCCCTCAGAAGCCTCAG
GACCCAACAGGTGGCAGGCAGTGGACAGGGTGCCCGCCCCACAGTGCTTTCCCCAG
CACCCCAGAGCCAGTCGGGACACCCCCCGCAGCCCGTCCTGGTGGCTGTGGAACTG
CACTGGGTGGCGGGCAGATGGTGGAAGGCAGCTTAGGAGACCTCACCAAAGAGAA
GATGGACCGGCTCTTGCTCCCAGCTCCTATTAGGCCCGGGGTGGGACCAGAGGTCAT
AGGTGCCCAACGGCAGCCAAACCAAAGACACTGGTGTGCATGGGGCAGCATGGTTG
TGCACGTGGGACCCTGGGGCGGACCCAGGAGCCAAACTCTTGAAGCACCCCCTGGG
TCAGGCCCAGCAGCGGAGTGGCCAGCCCCAGTTTCCCATTGCTCCTCTCTGCGGCCA
GGGCCAGGTGGGTTCATATTTACAGATATGCCCAGCCAGTCCTGGTCGGCCACACCA
GTGTCCCAAAGAGGAGAGCGCAGCAGAGCCAGGGGTCTGTTCTGTAGCAGCCACCC
CCCTGCCCCCACTCCAGGGCAGCCATGATGTGCTTGGGCCCACCAGGGCCTTCCGGG
CTGCTCTCTTCCCTGAGCCCGGAACCGGCGACGCACATGTGTCTTTTGTTGGTGTGTT
TGTTTTTTTCCAGGGAGGTCTAATTCCGAAGCAGTATTCCAGGTTTTCTCTTTGTTTT
ATCAGTGCCAAGATGACCTGTTGTGTCATATAATTTAAGCAGAGCTTAGCATTTATT
TTATTCTTTAGAAAACTTAAGTATTTACTTTTTTAAAGCTATTTTTCAAGGAACCTTTT
TTTGCAGTATTATTGAATTTATTTTCTAAATCAGGATTGAAACAGGAACTTTTCCAGG
TGGTGTTAATAAGCCATTCAAGTGCCTTACACAGCTTTGAAGAAACTAGGACTGCAG
TGGGCTCGGATAGGCCCATTGAGGTTTTTAGAAAAGCAGGATTTGTTTTGTTAGGGA
GGCATGATTTTGGTGAGATCTTTCTGGAAGAGTTTTCCGCCTCTTTGTGATGCTGAAC
ACCCCCAAGGTTCTCCCCTCCCCCCGCTGCCCAGGTGACTGGCAGGAGCTGCGACTG
CCACGTAGTGGTGCCTGGGCCCGACAGCGGGGCTCTGGGCATCCCGGGTGACCTTG
GCCCATCTGCCTGCATTCCCACCCCCTTGGGCCTGGCTGGATCCCAGGCAGAGGGAC
CTTGCTGCTGTGTGATTGGAACATTCCCAAATATCTTGTGAATTTGTAATCAAATTGG
TCTCATTGGGAAAGACTCTTAATTAAGAGGCTCAGGCAAGCACAGAGGCAGCCCGT
GGGTCTCTGTCTCAGTCTGGAGGCAGCAGGGATGCTGCTGGGAGTCCATGGCACAG
GCCACAGCCCCTCACCTTGCCGCGGTGGCTGGCAGCACGCCTGCCTTGCTCTGCCCC
ATGCCCTGAACAGGCATGAGAGCTCCACGTCCCCTAGTGCACCCTGAGAGGGGGCT
CACAAGTGACCGATCCTGGGTGCCTCAGGGAGCTCACTGAGGGCGTGCAAAGTTGA
AAGTGGCAAGGCTGGGGGAGGGTGTCGGGTAGAGGGAAGAGGGCAGGGGGCTAGG
GGAGGACTCAGAGGCCATCTGCAGGGCCAAGCCACAGGAAGGGCTGAGCTGGAGG
TGGGCAGGGCTGCTCCAGGCAGGTCAGAGCAGTGCAGGGGGAGGAGAGGAGAAAG
GGAGGAAGCTGGGCTGTGTGGTCCCCATGAAGGCATTCAGAGTCCACCTGCAGACA
GCGAGAGCCCCAGGAAGGTTTGCACAGCTGTGCCCCAAGCACCTTGGCCTCCTCTCA
GCTCGCCGAGGAGGCACGCTAGAGCCGCCTTCCCGGTGGGAGCCCTCTGTCCCACA
GGGAGCGGGGAGCCAGCTTTGCTGGGGCCCTACCTGCATGCCCAGCCTTACCCCTCA
TTCTCACAGCACAGATGAGGTTGAGACCATGCAGTCAATGCATTGCTTAAGGTCTCT
TATTTACAAAAAAAAACCTTAAACATAGTCGCTGTCATTCAGACATTCAGAGAATGG
TTGGCCACAAACAATGACCAAGTATTGCTTGGCTTAACTTGAAGGCCTGCTGTCTCC
TTCTGGGGGTCAGGGACGCAGCTCCACCCTCACCACTAGCCCACCCTGCCCGTGGGC
ATAACCTTGACGAAGAGAGAGAATGATTGGCATCTGCTTTTCTCTTTTCTTTGCTAAT
AATTCTGTTCCTGGCTGCCGAGAGTGAAGTTTCACCATGTGGAGGTTTGGCTCCTAT
CACCTGGTGGTCTGATTCATACCCTAGCCTGAGGCTCCACTGGAAGATCTCGCAGCC
TCAGTGTATGGGAAACCCTTTCCCCAGGCTTGTCCCAGCACTGCCGCTCCCCACCCC
TGAGCCAGGACCCCAGAGGATGGCCATGCCCCGTGCCTGGCAGAGGTCTGGTGCCA
GCACTGGGAGCTGCTCCGCCCTTGCCTTGGGGCCGAGGGAGCCCTCGTCCACCCCTG
CACAGCAGCTGGGCACAGAGGAGCGCTCTTCCATCTTGACCAGGACTGCACCAAGA
AGCACCAGGTGTCTTCAGCCTCCAACCTCCGGGGCGACCTTCTCTTCCAGCCACAGT
CCCATGAGGGCCCCTAGCCAGGGACACTGGTCTGTAAATTGTAATCCTTTCTCCAGC
CCAGCTCTCCACTTGTTCCTTGTGTGAGCTGAGCAGGCAGTGCACCTCTGAGTGTCC
CTTTTGTAAGGCCCAGGGGTTGCACTGAGTCTGCAGAGGCCGCGACCTCCTAGAACG
CTGTGGGTGCAGGTGAGCCGGCGTGTCCTGGGGAGATGCTGCCAGCACACAGGGGC
CCTCCTGCTGCCAGCAGGTTGGGGTGGTTAAGTCTTATTAGTGTCTATTCTTAAAATT
AAGTGGGCTGGAGAAGAATGGAGCTCCACATGCCAGCACCGTATATGGAATACAAA
AGCTGGGGAAGCAGGGCCTGCCTTACAGGTGTGGCTGACTCTGAGCCCAGGCCTGC
AGGGGTGGAGGGCAGTCCCTCAGAATCCCAGAGGCAGTCCCAGCCTCAGAACCCAG
GATAGGAAATGGGTGTGTTTAGTGGGGAAAGGGACGGGGTGCAGACGGCAGGGCC
AGTATGGGGCCCCCTCCCTCTCCTCTCCTCTCCTATGGTGAGCCCAGCGTGGGCACC
GGGCCGTCTCAGCCGTGTTCCCAGGGCTGGGAGGACAGCTCTGGCCCTTCTTAGGCC
TAGCCTCGTCCCAAGCTAAATGTAAGCCAGTTGGGCTGTGTTAAAGGAAGCAGTGTT
TTTGGTTCGATTCTGCCTCTGTAGCTCAAGGGGGGCAGCCCCCAGAGTCCTGTGCAT
TCTGCCAAGGCTCCATAGCTTTGCCAAATGCACGGAGCTCTGCCATTCCGGTGCAGT
GCAGGCCTTGCGAAGGGTTTATCTGCGTTCGTCTCGGTGGGCTTCTCCTGCATGGGA
GTTGTGTTCCTGTGCAAGGGGGAGCTTTGCTCCAGGACAGGATGACTGTCTTCCCTA
TTCTTAGGGACAAGTCCCAAGATGCCAGAAAGGCAGTCTCCCAAGGACCCACCATG
CAGAAGTGTCAATAAACCACAAGTTCTGAACTCTGTAAAAAAAAAAAAAA
PPP2R1A (SEQ ID NO: 19; CR450340.1).
ATGGCGGCGGCCGACGGCGACGACTCGCTGTACCCCATCGCGGTGCTCATAGACGA
ACTCCGCAATGAGGACGTTCAGCTTCGCCTCAACAGCATCAAGAAGCTGTCCACCAT
CGCCTTGGCCCTTGGGGTTGAAAGGACCCGAAGTGAGCTTCTGCCTTTCCTTACAGA
TACCATCTATGATGAAGATGAGGTCCTCCTGGCCCTGGCAGAACAGCTGGGAACCTT
CACTACCCTGGTGGGAGGCCCAGAGTACGTGCACTGCCTGCTGCCACCGCTGGAGTC
GCTGGCCACAGTGGAGGAGACAGTGGTGCGGGACAAGGCAGTGGAGTCCTTACGGG
CCATCTCACACGAGCACTCGCCCTCTGACCTGGAGGCGCACTTTGTGCCGCTAGTGA
AGCGGCTGGCGGGCGGCGACTGGTTCACCTCCCGCACCTCGGCCTGCGGCCTCTTCT
CCGTCTGCTACCCCCGAGTGTCCAGTGCTGTGAAGGCGGAACTTCGACAGTACTTCC
GGAACCTGTGCTCAGATGACACCCCCATGGTGCGGCGGGCCGCAGCCTCCAAGCTG
GGGGAGTTTGCCAAGGTGCTGGAGCTGGACAACGTCAAGAGTGAGATCATCCCCAT
GTTCTCCAACCTGGCCTCTGACGAGCAGGACTCGGTGCGGCTGCTGGCGGTGGAGG
CGTGCGTGAACATCGCCCAGCTTCTGCCCCAGGAGGATCTGGAGGCCCTGGTGATGC
CCACTCTGCGCCAGGCCGCTGAAGACAAGTCCTGGCGCGTCCGCTACATGGTGGCTG
ACAAGTTCACAGAGCTCCAGAAAGCAGTGGGGCCTGAGATCACCAAGACAGACCTG
GTCCCTGCCTTCCAGAACCTGATGAAAGACTGTGAGGCCGAGGTGAGGGCCGCAGC
CTCCCACAAGGTCAAAGAGTTCTGTGAAAACCTCTCAGCTGACTGTCGGGAGAATGT
GATCATGTCCCAGATCTTGCCCTGCATCAAGGAGCTGGTGTCCGATGCCAACCAACA
TGTCAAGTCTGCCCTGGCCTCAGTCATCATGGGTCTCTCTCCCATCTTGGGCAAAGA
CAACACCATCGAGCACCTCTTGCCCCTCTTCCTGGCTCAGCTGAAGGATGAGTGCCC
TGAGGTACGGCTGAACATCATCTCTAACCTGGACTGTGTGAACGAGGTGATTGGCAT
CCGGCAGCTGTCCCAGTCCCTGCTCCCTGCCATTGTGGAGCTGGCTGAGGACGCCAA
GTGGCGGGTGCGGCTGGCCATCATTGAGTACATGCCCCTCCTGGCTGGACAGCTGGG
AGTGGAGTTCTTTGATGAGAAACTTAACTCCTTGTGCATGGCCTGGCTTGTGGATCA
TGTATATGCCATCCGCGAGGCAGCCACCAGCAACCTGAAGAAGCTAGTGGAAAAGT
TTGGGAAGGAGTGGGCCCATGCCACAATCATCCCCAAGGTCTTGGCCATGTCCGGA
GACCCCAACTACCTGCACCGCATGACTACGCTCTTCTGCATCAATGTGCTGTCTGAG
GTCTGTGGGCAGGACATCACCACCAAGCACATGCTACCCACGGTTCTGCGCATGGCT
GGGGACCCGGTTGCCAATGTCCGCTTCAATGTGGCCAAGTCTCTGCAGAAGATAGG
GCCCATCCCGGACAACAGCACCTTGCAGAGTGAAGTCAAGCCCATCCTAGAGAAGC
TGACCCAGGACCAGGATGTGGACGTCAAATACTTTGCCCAGGAGGCTCTGACTGTTC
TGTCTCTCGCC
CFLAR (SEQ ID NO: 20; NM_003879.5).
ATACTCAGTCACACAAGCCATAGCAGGAAACAGCGAGCTTGCAGCCTCACCGACGA
GTCTCAACTAAAAGGGACTCCCGGAGCTAGGGGTGGGGACTCGGCCTCACACAGTG
AGTGCCGGCTATTGGACTTTTGTCCAGTGACAGCTGAGACAACAAGGACCACGGGA
GGAGGTGTAGGAGAGAAGCGCCGCGAACAGCGATCGCCCAGCACCAAGTCCGCTTC
CAGGCTTTCGGTTTCTTTGCCTCCATCTTGGGTGCGCCTTCCCGGCGTCTAGGGGAGC
GAAGGCTGAGGTGGCAGCGGCAGGAGAGTCCGGCCGCGACAGGACGAACTCCCCC
ACTGGAAAGGATTCTGAAAGAAATGAAGTCAGCCCTCAGAAATGAAGTTGACTGCC
TGCTGGCTTTCTGTTGACTGGCCCGGAGCTGTACTGCAAGACCCTTGTGAGCTTCCCT
AGTCTAAGAGTAGGATGTCTGCTGAAGTCATCCATCAGGTTGAAGAAGCACTTGATA
CAGATGAGAAGGAGATGCTGCTCTTTTTGTGCCGGGATGTTGCTATAGATGTGGTTC
CACCTAATGTCAGGGACCTTCTGGATATTTTACGGGAAAGAGGTAAGCTGTCTGTCG
GGGACTTGGCTGAACTGCTCTACAGAGTGAGGCGATTTGACCTGCTCAAACGTATCT
TGAAGATGGACAGAAAAGCTGTGGAGACCCACCTGCTCAGGAACCCTCACCTTGTTT
CGGACTATAGAGTGCTGATGGCAGAGATTGGTGAGGATTTGGATAAATCTGATGTGT
CCTCATTAATTTTCCTCATGAAGGATTACATGGGCCGAGGCAAGATAAGCAAGGAG
AAGAGTTTCTTGGACCTTGTGGTTGAGTTGGAGAAACTAAATCTGGTTGCCCCAGAT
CAACTGGATTTATTAGAAAAATGCCTAAAGAACATCCACAGAATAGACCTGAAGAC
AAAAATCCAGAAGTACAAGCAGTCTGTTCAAGGAGCAGGGACAAGTTACAGGAATG
TTCTCCAAGCAGCAATCCAAAAGAGTCTCAAGGATCCTTCAAATAACTTCAGGCTCC
ATAATGGGAGAAGTAAAGAACAAAGACTTAAGGAACAGCTTGGCGCTCAACAAGA
ACCAGTGAAGAAATCCATTCAGGAATCAGAAGCTTTTTTGCCTCAGAGCATACCTGA
AGAGAGATACAAGATGAAGAGCAAGCCCCTAGGAATCTGCCTGATAATCGATTGCA
TTGGCAATGAGACAGAGCTTCTTCGAGACACCTTCACTTCCCTGGGCTATGAAGTCC
AGAAATTCTTGCATCTCAGTATGCATGGTATATCCCAGATTCTTGGCCAATTTGCCTG
TATGCCCGAGCACCGAGACTACGACAGCTTTGTGTGTGTCCTGGTGAGCCGAGGAG
GCTCCCAGAGTGTGTATGGTGTGGATCAGACTCACTCAGGGCTCCCCCTGCATCACA
TCAGGAGGATGTTCATGGGAGATTCATGCCCTTATCTAGCAGGGAAGCCAAAGATG
TTTTTTATTCAGAACTATGTGGTGTCAGAGGGCCAGCTGGAGGACAGCAGCCTCTTG
GAGGTGGATGGGCCAGCGATGAAGAATGTGGAATTCAAGGCTCAGAAGCGAGGGCT
GTGCACAGTTCACCGAGAAGCTGACTTCTTCTGGAGCCTGTGTACTGCGGACATGTC
CCTGCTGGAGCAGTCTCACAGCTCACCATCCCTGTACCTGCAGTGCCTCTCCCAGAA
ACTGAGACAAGAAAGAAAACGCCCACTCCTGGATCTTCACATTGAACTCAATGGCT
ACATGTATGATTGGAACAGCAGAGTTTCTGCCAAGGAGAAATATTATGTCTGGCTGC
AGCACACTCTGAGAAAGAAACTTATCCTCTCCTACACATAAGAAACCAAAAGGCTG
GGCGTAGTGGCTCACACCTGTAATCCCAGCACTTTGGGAGGCCAAGGAGGGCAGAT
CACTTCAGGTCAGGAGTTCGAGACCAGCCTGGCCAACATGGTAAACGCTGTCCCTA
GTAAAAATACAAAAATTAGCTGGGTGTGGGTGTGGGTACCTGTATTCCCAGTTACTT
GGGAGGCTGAGGTGGGAGGATCTTTTGAACCCAGGAGTTCAGGGTCATAGCATGCT
GTGATTGTGCCTACGAATAGCCACTGCATACCAACCTGGGCAATATAGCAAGATCCC
ATCTCTTTAAAAAAAAAAAAAAAGGACAGGAACTATCTTACTCAATGTATTAGTCAT
GTTTCTCTAGAGGGACAGAACTAATAGGATACATGTATATAAAAAGGGGAGTTTATT
AAGGAGTATTGACTCACATGATCACAGGGTTAGGTCCCACAATAGGTCATCTGCAA
GCAAGGAAGCCAATTCAAGTCCCAAAGCTGAAGAACTTGGAGTCCAATGTTTGAGG
GCAGGAAGCATTCAGCATGAGAGAAAGATGGAGGCCAGAAGACTACACCAGTCTA
GTCTTTCCATGTTTTGCCTGCTTTTATTCTGGCAGTGCTGGCAGCTGATTAGATGGTG
CCCACCCAGATTGAGGATGGTCTGCCTTTCCCAGTCCACTGACTCAAATGTTAAATC
TCCTTTGGCAGCACCCTCACAGATGTACCCGGGAACACTTTGCATCCTTCTATTCAAT
CAAGTTGATACTCAGTATTAACCATCACAGTCCATTTGGGCAACTATACCAAATTAC
CATAGACCAGGTGACTTAAACAGCAGTTATTTCTCACAGTTCCGGAGGCTGGGAAAT
CCAACATCTAAGTGGTAGCATATCTGGTGTCTGGTAAGGCATGCTTCCAGATCTTAC
CAGATGTCAGTCTTTTGATGTTCTCACATGGCAGAAAAAGAGGATGCAAACTCTCAA
GTATATCTTTAAGGGCACAAATTCCATTCATGAGGGCTCTACCCTCATCACCTAATT
ACCTCCCAAAGGCCCCACCTTCTGATACTGTCACTTTGGGGATACTGTCTCCCCTTTG
AATTCTGGGGGGAATACAAACATTCAGTTTGTAACAATAGCCTTATGATTTAGAGGT
TACTTGTTCATTCACCTAGACCTCAAATTGCATTTTACAGCTAGTCAAGTATATCTTT
CTCTGATTTGATAGTGTGACCTAAAAGGGGACCATTGTTTGAAATATCATTAGAGTT
GCTTATTATTATTATTATTATTATTATTATTATTATTATTATTATTATTGAGACAGAGT
TTCATTCTGCTGCCCAGGCTGGAGTGCAGTGGCATCATCTTGGCTCATTGCAACCTCT
GCCTTCTGGGTTCAAGCGATTCTCCTGCCTCAGCCTCCCGAGTAGCTGGGATTACAG
GCTCCTGCCACCACACCCGGCTAATTTTTGTATTTTTAGTGGAGACAGGGTTTCCACC
ATGTTGGCCAGCGTGGTCTTGAACTCCTGACCTCAGGTGATTCACCAGCCTCGGCCT
CCCAAAGTGCTGGGATTACAGGTGTGAGCCACTGCACCTGGCCTATTATTATTTTTA
AATTTTTTTTTTTTAATTGATCATTCTTGGGTGTTTCTCACAGAGGGTGATTTGGCAG
GGTCACAGGACAATAGTGGAGGGAAGGTCAGCAGATAAACAAGTGAACAAAGGTC
TCTGGTTTTCCTAGGCAGAGGACCCTGCGGCCTTCCGCAGTGTTTGTGTCCCTGGGTA
CTTGAGATTAGGGAGTGGTGATGACTCTTAAGGAGCATGCTGCCTTCAAGCATCTGT
TTAACAAAGCACATCTTGCACTGCCCTTAATCCATTTAACCCTGAGTGGACACAGCA
CATGTTTCAGAGAGCACAGGGTTGGGGGTAAGGTCATAGATCAACAGCATCCTAAG
GCAGAAGAATTTTTCTTAGTACAGAACAAAATGAAGTCTCCCATGTCTACTTCTTTCT
ACACAGACACAGCAACAATCTGATTTCTCTATCTTTTCCCCACCTTTCCCCCTTTTCT
ATTCCACAAAACCGCCATCGTCATCATGGCCTGTTCTCAATGAGCTGTTGGGTACAC
CTCCCAGACGGGGTGGCGGCTGGGCAGAGGGGCTCCTCACTTCCCAGATGGGGCGG
CCAGGCGGACGCGCCCCCCACCTCCCTCCCGGACGGGATAGCTGGCCGGGCGGGGG
CTGACCCCCCACCTCCCTCCCCGACGGGGCGGCTGGCCGGGCGGGGGCTGACCCCC
ACGCCTCCCTCCCGGACGGGGCGGCTGCCAGGCGGAGGGGCTCCTCACTTCTCAGA
CGGGGTGGCTGCTGGGCGGAGACGCTCCTCACTTCCCAGACAGGGTGGCTGTCGGG
CGGAGGGGCTCCTCACTTCTCAGACGGGGCAGCTGCGGGCGGAGGGGCTCCTCACT
TCTCAGACGGGGTGGCCGGGCAGAGAAGCTCCTCACATCCCAGACGGGGGGGCGGG
GCAGAGGCGCTCCCCACATCTCAGACGATGGGCGGCCGGGCAGAGACGCTCCTCAC
TTCATCCCAGACGGGGTGGCGGCCGGGCAGAAGCTGTAATCTCGGCACCCTGGGGG
GCCAAGGCAGGCGGCTGGGAGGCGGAGGCCGTAGCCAGCTGAGATCACACCACTGC
ACTCCAGCCTGGGCAACATTGAGCACTGAGTGGACGAGACTCTGCCCGCAATCCCG
GCACCTCGGGAGGCCGAGGCTGGCAGATCACTCGCAGTCAGGAGCTGGAGACCAGC
CCGGCCAACACAGTGAAACCCTGTCTCCACCAAAAAAATACGAAAACCAGTCAGGC
GTGGCGGCGCCCGCAATGGCAGGCACGCGGCAGGCCGAGGCGGGAGAATCAGGCA
GGGAGGCTGCAGTGAGCCGAGATGGCAGCAGTACAGTCCAGCTTCGGCTCGGCATC
AGAGGGAGACCGTGGGGAGAGGGAGAAGAGAGGGAGGGGGAGAGGGCTATTTTTA
AAATTTTTTAAAATTGCTGAACAGGGGTACCTCTGGGCAGTGTGTCAGAATACCACT
TTTTAAATATTTTATGATTTATTTATTTTTCTATTTCTTGAGGTTTTAACTGATGTGTA
TCTGTATGTCTATTTGTGTATATTTTGTCATGATCATGTAACAGAGTCTGAAAAGTGT
CGAAGAGACAGTTTTCAGGAACAACAAGCAATTATTCCTACTTTCCAAGTTATTTTG
ATGCCATGGTGGCTCATACCTATAATCTGAGTACTTTGGGAGGCTGAGGTGGACTGA
TCACTTGAGCCCAGGAGTTTGAGACCAGCCTGGGCAACATAGCAAGACTCCATCTCT
ACAAAAAAAGACAAAATTTAGCTGAGCGTGGTGGCGTGTTCCTGTAGTCCCAGCTA
CTTGGGAGGCTGAAGTGAGTGGATCCCCTGAGCCCAGAGAGGTCAAGGTTGTGATG
AGCTGTGATCACACCACTGCACTTCAGCATGGGAGACAGAGTGAGACCCTGTTTCAG
AAAAAATAAATAAATAAAACCACCAGCACCACAAACAACAACAAAAAGTTATTTTG
TACTTGTTTTGAGCACAGGACTCCTGAGGGTATCTTTGCATTTAATATTACATAGGG
GTGCCAGTGGGAAGTAATGTGTATGCTTGGCCTCATGAGCTAAAACCCTGTGTTAAT
TATGACAGAAGGAAAGTGTGTGAGAGAGATCTTAACTACCTAGCAGCTCTAGCTGC
CATCTTGAACCATGAAGATACGGGCCACACGTAGGGGTAGCTGGGTAGTGAGCAGC
AAGAAGCCTTGTTGGATGAGGGCACGAAGGAGCAGAATCACTGGAATCACTGTGTC
AGCCCTAATTACCTACCTCTGGACTTTTATGTGAGGGGAAAAAAAATTGACAGTTTA
TATTTATCTCAACCTAGTTAACCCAAGTGATGCATTGTTATGAGATTAAAATGTTTGG
AGGCCGGGTGCGGTGGCTCACGCCTATAATCCCAGCCCTTTGGGAGGCCAAGGCGG
GCGGATCACGAGGTCAGGAGATCAAGACCATCCTGGCTAACATGTAAAACCCCGTC
TCTACTAAAAATACAAAAAATTAGCCAGGCGTTGTGGCGGTCGCCTGTAGTCCCTGC
TATTTGGGAGGCCGAGGCAAGAGAACGGCATGAACCTGGGAGGTGGAGCTTGCAGC
GAGCTGAGATCTTGCCACTGCACTCCAGCCTGGGCGACAGTGCGAGACTCTGTCTCA
AAAATAAATAAATAAATAAATAATAAATAAAATGTTTGGAATGTTGGCTTCATCCCT
GGGATGCAAGGCTGGTTCAACATACGCAAATCAAGAAACATAATTCATCACATAAA
CAGAACTAAAGACAAAAACCACATGATTATCTCAATAGATACAGAAAAGGCCTTCA
ATAAAATTCAACGTTGCTTCATGTTAAAAACTCTCAATAAACTAGGTATTGATGGAA
AATATCTCAAAATAATAACCATTTATGACAAACCCACAGCCATTATCATACTGAATG
GGCAAAAGCTGGAAGCATTCCCCTTGAAAACTGGCACAAGACAGGGATGCCGTCTC
ACCACTCCTATTTAACATAGTATTGGAAGTTCTGGCCAAGAAAATCAGGCAAGAGA
AACAAATAAGGGGTATTCAAATAGGAAAAGAGGAAGTAAAACTGTGTTTGCAGATG
ACATGATACTATATCTAGAAAACCCCATTATCTCCACCCAAAAGTTCCTTAAGCTGA
TAAGCAACTTCAGCAAAGTCTCAGGATACAAAATCAATGTGCAGAAATCACAAGCA
TTCTATACACCAACAATACACAAGCAGAGAGCCAAATCATGAATGAACTCCCATTC
ACAGTTGCTAGAAAGAGAATAAAATACCTAGGAATACAGCTAATAAGATGTGAAGG
ATCTCTTCAAGGAGAACTACAAACCACTGCTCAAGGAAATAAGAGAGGACACAAAT
GAAAAAACATTCCATTCTCGTGGATAGGAAGAATCAATATCATGAAAATGGCCATA
CTACCCAAAGTAATTTATAGGTTCATTGCTATTCCCATTAAACTACTATTGACATTCT
TCACAGAATTAGAAAAAAACTACTTTAAAATTCAAATGGAACCAAAAAAGAGCCCG
TATAACCAAGACAACAATAAGCAAAAAGAACAAAGCTGGAAGCATCACACTACCC
AACTTCAAAGTATACTGCAAGGCTACAGTAGCCAAAATGGCATGGTACTGGTACAA
AAACAGACACATAGACCAATGGAACAGAATAGAGACCAGAGAAAGAAGACCACAC
ATCTACAGCCATCTGATCATCGACAAACCTGACAAAAACAAGCAATGGGGAAAAGA
TTCCCTATTTAATAAATGGTGCTGGGAAAACTGGCTAGCCATATGCAGAAAATTGAA
ACTGACCCCTTCCTTACACCTTATACAAAAATTAACTCAAGATTAAAGACTTAATGT
AAAACCTAAAACTATAAAAACCCTAGAAGAAAATCTATTTAATACCATTCAAGACA
TAGGCACAAGCAAAGGTTTCATGACAAAAACATCAAAAGCAATTGCAACAAAAGCA
AAAATTACAAATGGGATCTAATTAAACTAAAGAGCTCCTGCACAGCAAAAGAAACT
ATCATTAGAGTGAACAGGCAACCTACAGAATGGGAGAACATTTTTGCAATCTATCCA
TCTGACAAAGGTCTAATATCCAGAACCTACAAGGAACTTAAAACAAATTTACAAGG
AAAAAAACAACCCCATCAAAAAGTGGACAAAGGACATGAACAGACACTTCTCAAA
AGAAGACATTTATGTGGCCAACAAACATATAAAAAAAAGCTCAACCTTACTGATCA
TTAGAGAAATGCAAAGGAGAACCACAATGAGATACCATCTCATGCCGGTCAGAATG
GTGATTATTAAAAAGTCAAAAAACAACAGATGCTGGCGAGGCTGTGGAGAAGTAGG
AACACTTTTACATTGTTGGTGGGAATGTAAATTAGTTCAACCGTTGTGGAAGTGTGT
GTGGCTATTCCTCAAAGATCTAGAACTAGAAATACTATTTGTCCCAGCAATCCCATT
ACTGGGTATATACCCAAAGGAATATAAACCATTTTATTATAAAGATACATGCACATT
TTTGTTCATTGCAGCACTCTTCACAATAGCAAAGACACAATAGCAAATGCCCATCAA
AGATAGACTGGATAAAGAAAATGTGGTACATATACACCATGGAATACTGTGCAGTG
CAGCCATTACAGCTTTTGGTGATACAGTGAATCAGATTTTTCATTAATTCTTTTAATT
GGTTATTACTGAACGTGAAAAAGTAATGTTTGTATTGAAATCTTGAGTCTGGCCATG
TTTCTATTTTAAATTCATAAAGAATTCTAACAAGAGGAATTCCAAGAATGTCATAAA
TGGATGTTTCTCCATGGATGAAGGAACTGTTTTATTCACTTGCTGATAATTCAGCCTA
ATCCAGTTTGACATCATATAGATAAGTAGTTGAATTATGGATTTAAAATACATATCA
TTTTCTAACTCCAAAGGTAATACTTATTTAAATGGTTTTGAAAATATAGAAAGGCAC
AATTTCTTTTTAAATCTGTTATTCTCCACCACCACTCAATCTGTCTATCATCTATCTCT
CCATTCATTCTTCCATTTGTTTATATCTGTTAATCTTTGTATGTGTTCATGTATAGCTT
TTACATGATTGGAATCATAATGCATATTCCATTTTGAAGTCTGCTTTTTTTTACACAA
AAATATGTTGTGAATATTTTCCTATATTATGAAATATCATTAGCTGAGCTTTTAGAAT
TGACTGCATGTTTTGGTACCATTTAGATATAGTTTAAGATACTTAGAAGTTATGTGGC
TTTGCCACTATGGATGAATCTTATTTACTCAATATTAATTACTTACAAATAACCTCAC
CTAAACACTACTCAGCCATAAAAAGGAATGAATTAATGACATTCACAGCAACCTGG
AGACTATTACTCTAAAGGAAGTAACTGAGGAATGGAAAACCAAACATTGTATGTTC
TCACTCATAAGTGGGAGATAAGCTATGAGGATGCAAAGGCATAAGAAGGATACAAT
GGACTTTGGGGACTTAGGGGAAAGGGTGGGAGGGGGGTGAAGGATAAAAGAATAC
AAATTGGGTTCAGTGTATACTGCTCAGGTGATGGGTGCACCAGAATCTCACAAGTAA
CCACTTAATTACTTACGCATGTAACCAGATACCACCTGTTCCCCAAACACCTATGGA
AATAATTTTGTTTTTTTTTTTAAAAAAGGAATGAGATCATGTCCTTTGCAGGGACATG
GATGAAGCTGGAAGCCATTATCCTCAGCAAACTAACAGAGGAGCAGGAAACCAAAC
ACCACATGTTCTCACTTGTAAGCGGAAGCTGAACAATGAGAACACACGGACACAGG
GATGAGATCAACACACACTGGGGCCTGATGCAGGGGCCGTAGCGGGGAGAGCATCA
GGATAACTAGCTAATGCATGTGGGGCTTAATACCTAGGTGATAGGTTGATAGGTGCA
GCAAACCACCATGGGACACGTTTACCTATGTAACAAACCCGCACATCCTGCACTTGT
ATCCAGAACTTAAAATATTTTAAAAATCTTTAGAGAATACAAAAAAAAAAAAAAAG
ATTCTTCAATGCATACACAATAAAATTGCAGTTCAGTCAAACATTGGAAGTCTTTCT
CTGACTGTCTAGTTGGTATCTTCATTTTCAGCTTCTTCAAGATCCCACTCCAAACACT
GTTAGCTCAGCCAAATTGAACAGCTCATATCTCCTACCTCTGGATCTTTGGTTCTGGT
GATTGTATATTTCTGGACCATCTGGAACCCCAGCATATCACCCTACCCCACATCTCC
ACATCCCCAAAATATAACCATACTTCAAGGGCAGTTCAAATACCATCTCCTTCTATC
CTCCATGAAGTCAGTTATCTCTTCCATTGGAATTATCGCCCCCTCTCCTGAACAGTAC
TATTTCGTGTGAATCTCCTCCAAGCCTTCTTTTCATTTTATATCTCATGCTGTAATTCT
TGGAAAGTATGCTGTAGCTCAAGTGCAGAATTCTCATCAGTTTTATCTTTATATCTCT
CCTAAACACTTTACCTGATGAAGAGCCTGGCATACACATAAATATATATTGAATGAA
TCAGTGATGGATTGAAAAGAGAAATGATGGATCTCCTAAATTTTAACTTTTATAAAA
TATTTTGATACATTCATGACCTTACTTTAGCAAGCAATGAACGTGATGTAAACTATT
GTTGATATAGTTTTTATATTGGAAGTGTAAGTAGTTTGTGGCATGGGATTGTGACAT
ATCCTAGGTTTCCTCATCTTCTTTTTATTGAAATGTAATTCACAAGCCATAAAATTTG
CCCCTTTAAAGTAAATGATGCAGTGGATTTTAGTATATTTACAGAGTTGTGCAATCA
TCACCACTATCTAATTCCAGAACATTTCCATCTACCTAGAAACTCCATACCAGTGAG
CTGCCACTCTAATCCTCCTCTTCCCCCAGCCTCTAGAAACAATAATCCATTTTCTGTC
TCTATGATTTGCCTGTTCTAGATATTTTATAAAAATAAACATGTGGCCTTTCGTGTCT
GACTTCCTTCACTTAAAAAAAAAAAAAAAAAA
DHRS13 (SEQ ID NO: 21; NM_144683.3).
CGCCTCCGCCTTCGGAGGCTGACGCGCCCGGGCGCCGTTCCAGGCCTGTGCAGGGC
GGATCGGCAGCCGCCTGGCGGCGATCCAGGGCGGTGCGGGGCCTGGGCGGGAGCCG
GGAGGCGCGGCCGGCATGGAGGCGCTGCTGCTGGGCGCGGGGTTGCTGCTGGGCGC
TTACGTGCTTGTCTACTACAACCTGGTGAAGGCCCCGCCGTGCGGCGGCATGGGCAA
CCTGCGGGGCCGCACGGCCGTGGTCACGGGCGCCAACAGCGGCATCGGAAAGATGA
CGGCGCTGGAGCTGGCGCGCCGGGGAGCGCGCGTGGTGCTGGCCTGCCGCAGCCAG
GAGCGCGGGGAGGCGGCTGCCTTCGACCTCCGCCAGGAGAGTGGGAACAATGAGGT
CATCTTCATGGCCTTGGACTTGGCCAGTCTGGCCTCGGTGCGGGCCTTTGCCACTGC
CTTTCTGAGCTCTGAGCCACGGTTGGACATCCTCATCCACAATGCCGGTATCAGTTC
CTGTGGCCGGACCCGTGAGGCGTTTAACCTGCTGCTTCGGGTGAACCATATCGGTCC
CTTTCTGCTGACACATCTGCTGCTGCCTTGCCTGAAGGCATGTGCCCCTAGCCGCGT
GGTGGTGGTAGCCTCAGCTGCCCACTGTCGGGGACGTCTTGACTTCAAACGCCTGGA
CCGCCCAGTGGTGGGCTGGCGGCAGGAGCTGCGGGCATATGCTGACACTAAGCTGG
CTAATGTACTGTTTGCCCGGGAGCTCGCCAACCAGCTTGAGGCCACTGGCGTCACCT
GCTATGCAGCCCACCCAGGGCCTGTGAACTCGGAGCTGTTCCTGCGCCATGTTCCTG
GATGGCTGCGCCCACTTTTGCGCCCATTGGCTTGGCTGGTGCTCCGGGCACCAAGAG
GGGGTGCCCAGACACCCCTGTATTGTGCTCTACAAGAGGGCATCGAGCCCCTCAGTG
GGAGATATTTTGCCAACTGCCATGTGGAAGAGGTGCCTCCAGCTGCCCGAGACGAC
CGGGCAGCCCATCGGCTATGGGAGGCCAGCAAGAGGCTGGCAGGGCTTGGGCCTGG
GGAGGATGCTGAACCCGATGAAGACCCCCAGTCTGAGGACTCAGAGGCCCCATCTT
CTCTAAGCACCCCCCACCCTGAGGAGCCCACAGTTTCTCAACCTTACCCCAGCCCTC
AGAGCTCACCAGATTTGTCTAAGATGACGCACCGAATTCAGGCTAAAGTTGAGCCTG
AGATCCAGCTCTCCTAACCCTCAGGCCAGGATGCTTGCCATGGCACTTCATGGTCCT
TGAAAACCTCGGATGTGTGCGAGGCCATGCCCTGGACACTGACGGGTTTGTGATCTT
GACCTCCGTGGTTACTTTCTGGGGCCCCAAGCTGTGCCCTGGACATCTCTTTTCCTGG
TTGAAGGAATAATGGGTGATTATTTCTTCCTGAGAGTGACAGTAACCCCAGATGGAG
AGATAGGGGTATGCTAGACACTGTGCTTCTCGGAAATTTGGATGTAGTATTTTCAGG
CCCCACCCTTATTGATTCTGATCAGCTCTGGAGCAGAGGCAGGGAGTTTGCAATGTG
ATGCACTGCCAACATTGAGAATTAGTGAACTGATCCCTTTGCAACCGTCTAGCTAGG
TAGTTAAATTACCCCCATGTTAATGAAGCGGAATTAGGCTCCCGAGCTAAGGGACTC
GCCTAGGGTCTCACAGTGAGTAGGAGGAGGGCCTGGGATCTGAACCCAAGGGTCTG
AGGCCAGGGCCGACTGCCGTAAGATGGGTGCTGAGAAGTGAGTCAGGGCAGGGCA
GCTGGTATCGAGGTGCCCCATGGGAGTAAGGGGACGCCTTCCGGGCGGATGCAGGG
CTGGGGTCATCTGTATCTGAAGCCCCTCGGAATAAAGCGCGTTGACCGCCGAAAAA
AAAAAAAAAAAAAAA
ACAA1 (SEQ ID NO: 22; NM_001607.3).
GGGTTCCCAGGCCGACTCTCCTTGTGGTTGGCTGAGGCTGGAGGTGGACGGGACTTT
TGGAGGGTCGCTCGCGTCTGTTCGCAGAGCTGTGGGCGGAGTTGAGGCCTTGGAGG
CTGAGATGTGGTTCTGCGCGTGTGCGGACGGCTGTCTGTTAACTCCGCGGTCAGTTC
CCGGACTGGTGGCTGGTCTGCAGGGTTGACCTGCGCAATGCAGAGGCTGCAGGTAG
TGCTGGGCCACCTGAGGGGTCCGGCCGATTCCGGCTGGATGCCGCAGGCCGCGCCTT
GCCTGAGCGGTGCCCCGCAGGCCTCGGCCGCGGACGTGGTGGTGGTGCACGGGCGG
CGCACGGCCATCTGCCGGGCGGGCCGCGGCGGCTTCAAGGACACCACCCCCGACGA
GCTTCTCTCGGCAGTCATGACCGCGGTTCTCAAGGACGTGAATCTGAGGCCGGAACA
GCTGGGGGACATCTGTGTCGGAAATGTGCTGCAGCCTGGGGCCGGGGCAATCATGG
CCCGAATCGCCCAGTTTCTGAGTGACATCCCGGAGACTGTGCCTTTGTCCACTGTCA
ATAGACAGTGTTCGTCGGGGCTACAGGCAGTGGCCAGCATAGCAGGTGGCATCAGA
AATGGGTCTTATGACATTGGCATGGCCTGTGGGGTGGAGTCCATGTCCCTGGCTGAC
AGAGGGAACCCTGGAAATATTACTTCGCGCTTGATGGAGAAGGAGAAGGCCAGAGA
TTGCCTGATTCCTATGGGGATAACCTCTGAGAATGTGGCTGAGCGGTTTGGCATTTC
ACGGGAGAAGCAGGATACCTTTGCCCTGGCTTCCCAGCAGAAGGCAGCAAGAGCCC
AGAGCAAGGGCTGTTTCCAAGCTGAGATTGTGCCTGTGACCACCACGGTCCATGATG
ACAAGGGCACCAAGAGGAGCATCACTGTGACCCAGGATGAGGGTATCCGCCCCAGC
ACCACCATGGAGGGCCTGGCCAAACTGAAGCCTGCCTTCAAGAAAGATGGTTCTAC
CACAGCTGGAAACTCTAGCCAGGTGAGTGATGGGGCAGCTGCCATCCTGCTGGCCC
GGAGGTCCAAGGCAGAAGAGTTGGGCCTTCCCATCCTTGGGGTCCTGAGGTCTTATG
CAGTGGTTGGGGTCCCACCTGACATCATGGGCATTGGACCTGCCTATGCCATCCCAG
TAGCTTTGCAAAAAGCAGGGCTGACAGTGAGTGACGTGGACATCTTCGAGATCAAT
GAGGCCTTTGCAAGCCAGGCTGCCTACTGTGTGGAGAAGCTACGACTCCCCCCTGAG
AAGGTGAACCCCCTGGGGGGTGCAGTGGCCTTAGGGCACCCACTGGGCTGCACTGG
GGCACGACAGGTCATCACGCTGCTCAATGAGCTGAAGCGCCGTGGGAAGAGGGCAT
ACGGAGTGGTGTCCATGTGCATCGGGACTGGAATGGGAGCCGCTGCCGTCTTTGAAT
ACCCTGGGAACTGAGTGAGGTCCCAGGCTGGAGGCGCTACGCAGACAGTCCTGCTG
CTCTAGCAGCAAGGCAGTAACACCACAAAAGCAAAACCACATGGGAAAACTCAGC
ACTGGTGGTGGTGGCAGTGGACAGATCAAGGCACTTCAACTCATTTGGAAAATGTG
AACACTGATGACATGGTATAGGAGTGGGTGGGGTGTTGAGCCACCCATCAGACCCT
CTTTAGCTGTGCAAGATAAAAGCAGCCTGGGTCACCCAGGCCACAAGGCCATGGTT
AATTCTTAAGGCAAGGCAAATCCATGGATGAGAAGTGCAATGGGCATAGTAAAAGT
GCATGAATTTATCTTAAAAAAAAAAAAAAAAAAAAAA
INPP5J (SEQ ID NO: 23; NM_001284285.1).
CAGGTTGAAATGGCTGATGACATCACTGGTTCCCGGGAGCGGTAGAGCTGGAGCCG
GAGCCAAGGGAGTCCAGGCTGCCGGGGGCTGCAGACATGGAGGGCCAGAGCAGCA
GGGGCAGCAGGAGGCCAGGGACCCGGGCTGGCCTGGGTTCCCTGCCCATGCCCCAG
GGTGTTGCCCAAACTGGGGCACCCTCCAAGGTGGACTCAAGTTTTCAGCTCCCAGCA
AAGAAGAACGCAGCCCTAGGACCCTCGGAACCAAGGTTGGCTCTGGCACCTGTAGG
GCCACGGGCAGCTATGTCAGCTTCCTCGGAAGGACCGAGGCTGGCTCTGGCATCTCC
CCGACCAATCCTGGCTCCACTGTGTACCCCTGAAGGGCAGAAAACAGCTACTGCCC
ACCGCAGCTCCAGCCTGGCCCCAACATCTGTGGGCCAGCTGGTGATGTCTGCCTCAG
CTGGACCAAAGCCTCCCCCAGCGACCACAGGCTCAGTTCTGGCTCCGACGTCCCTGG
GGCTGGTGATGCCTGCCTCAGCAGGGCCAAGATCTCCCCCAGTCACCCTGGGGCCCA
ATCTGGCCCCAACCTCCAGAGACCAGAAGCAGGAGCCACCTGCCTCCGTGGGACCC
AAGCCAACACTGGCAGCCTCTGGCCTGAGCCTGGCCCTGGCTTCTGAGGAGCAGCC
CCCAGAACTCCCCTCCACCCCTTCCCCGGTGCCCAGTCCAGTTCTGTCTCCAACTCAG
GAACAGGCCCTGGCTCCAGCATCCACGGCATCAGGCGCAGCCTCTGTGGGACAGAC
ATCAGCTAGAAAGAGGGATGCCCCAGCCCCTAGACCTCTCCCTGCTTCTGAGGGGC
ATCTCCAGCCTCCAGCTCAGACATCTGGTCCTACAGGCTCCCCACCCTGCATCCAAA
CCTCCCCAGACCCTCGGCTCTCCCCCTCCTTCCGAGCCCGGCCTGAGGCCCTCCACA
GCAGCCCTGAGGATCCTGTTTTGCCACGGCCACCCCAGACCTTGCCCTTGGATGTGG
GCCAGGGTCCTTCAGAGCCTGGCACTCACTCCCCTGGACTTCTGTCCCCCACCTTCC
GGCCTGGGGCCCCCTCAGGCCAGACTGTGCCCCCACCTCTGCCCAAGCCACCCCGAT
CACCCAGCCGTTCCCCAAGCCACTCCCCGAATCGCTCTCCCTGTGTTCCCCCAGCCC
CTGACATGGCCCTCCCAAGGCTTGGCACACAGAGTACAGGGCCTGGCAGGTGCCTG
AGCCCCAACCTTCAGGCCCAAGAAGCCCCAGCCCCAGTCACCACCTCCTCTTCTACA
TCCACCCTGTCATCCTCCCCTTGGTCAGCTCAGCCTACCTGGAAGAGCGACCCCGGC
TTCCGGATCACTGTGGTCACATGGAACGTGGGCACTGCCATGCCCCCAGACGATGTC
ACATCCCTCCTCCACCTGGGCGGTGGTGACGACAGCGACGGCGCAGACATGATCGC
CATAGGGTTGCAGGAAGTGAACTCCATGCTCAACAAGCGACTCAAGGACGCCCTCT
TCACGGACCAGTGGAGTGAGCTGTTCATGGATGCGCTAGGGCCCTTCAACTTCGTGC
TGGTGAGTTCGGTGAGGATGCAGGGTGTCATCCTGCTGCTGTTCGCCAAGTACTACC
ACCTGCCCTTCCTGCGAGACGTGCAGACCGACTGCACGCGCACTGGCCTGGGCGGCT
ACTGGGGTAACAAGGGTGGCGTGAGCGTGCGCCTGGCGGCCTTCGGGCACATGCTC
TGCTTCCTGAACTGCCACTTGCCTGCGCATATGGACAAGGCGGAGCAGCGCAAAGA
CAACTTCCAGACCATCCTCAGCCTCCAGCAGTTCCAAGGGCCGGGCGCACAGGGCA
TCCTGGATCATGACCTCGTGTTCTGGTTCGGGGACCTGAACTTCCGCATTGAGAGCT
ATGACCTGCACTTTGTCAAGTTTGCCATCGACAGTGACCAGCTCCATCAGCTCTGGG
AGAAGGACCAGCTCAACATGGCCAAGAACACCTGGCCCATTCTGAAGGGCTTTCAG
GAGGGGCCCCTCAACTTCGCTCCCACCTTCAAGTTTGATGTGGGTACCAACAAATAC
GATACCAGTGCCAAGAAACGGAAGCCAGCTTGGACAGACCGTATCCTATGGAAGGT
CAAGGCTCCAGGTGGGGGTCCCAGCCCCTCAGGACGGAAGAGCCACCGACTCCAGG
TGACGCAGCACAGCTACCGCAGCCACATGGAATACACAGTCAGCGACCACAAGCCT
GTGGCTGCCCAGTTCCTCCTGCAGTTTGCCTTCAGGGACGACATGCCACTGGTGCGG
CTGGAGGTGGCAGATGAGTGGGTGCGGCCCGAGCAGGCGGTGGTGAGGTACCGCAT
GGAAACAGTGTTCGCCCGCAGCTCCTGGGACTGGATCGGCTTATACCGGGTGGGTTT
CCGCCATTGCAAGGACTATGTGGCTTATGTCTGGGCCAAACATGAAGATGTGGATGG
GAATACCTACCAGGTAACATTCAGTGAGGAATCACTGCCCAAGGGCCATGGAGACT
TCATCCTGGGCTACTATAGTCACAACCACAGCATCCTCATCGGCATCACTGAACCCT
TCCAGATCTCGCTGCCTTCCTCGGAGTTGGCCAGCAGCAGCACAGACAGCTCAGGCA
CCAGCTCAGAGGGAGAGGATGACAGCACACTGGAGCTCCTTGCACCCAAGTCCCGC
AGCCCCAGTCCTGGCAAGTCCAAGCGACACCGCAGCCGCAGCCCGGGACTGGCCAG
GTTCCCTGGGCTTGCCCTACGGCCCTCATCCCGTGAACGCCGTGGTGCCAGCCGTAG
CCCCTCACCCCAGAGCCGCCGCCTGTCCCGAGTGGCTCCTGACAGGAGCAGTAATG
GCAGCAGCCGGGGCAGTAGTGAAGAGGGGCCCTCTGGGTTGCCTGGCCCCTGGGCC
TTCCCACCAGCTGTGCCTCGAAGCCTGGGCCTGTTGCCCGCCTTGCGCCTAGAGACT
GTAGACCCTGGTGGTGGTGGCTCCTGGGGACCTGATCGGGAGGCCCTGGCGCCCAA
CAGCCTGTCTCCTAGTCCCCAGGGCCATCGGGGGCTGGAGGAAGGGGGCCTGGGGC
CCTGAGGGTGGGGTAGGCAGATGGGCCAAGGTGACCACCATTCTGCCTCAATCTTTT
GCAAGCCCACCTGCCTCTCTCCTGCTGCTCCTCCAGCTGTATCTGCACCTGCCTCTCT
GTCCTGGCCAGGGGTGGACAACTGGGGTCCCCCAAAACTCAGTCCTGGCACCTCAA
CTGTGACAATCAGCAAAGCCCCACCCAGGCCCCCATCTGGGATGATGGGAGAGCTC
TGGCAGATGTCCCAATCCTGGAGGTCATCCATTAGGAATTAAATTCTCCAGCCTCAA
AAAAAAAAAAAAAAA
OAZ1 (SEQ ID NO: 24; NM_004152.2).
TTTTGCGAACGGCGAGCAGCGGCGGCGGCGCGGAGAGACGCAGCGGAGGTTTTCCT
GGTTTCGGACCCCAGCGGCCGGATGGTGAAATCCTCCCTGCAGCGGATCCTCAATAG
CCACTGCTTCGCCAGAGAGAAGGAAGGGGATAAACCCAGCGCCACCATCCACGCCA
GCCGCACCATGCCGCTCCTAAGCCTGCACAGCCGCGGCGGCAGCAGCAGTGAGAGT
TCCAGGGTCTCCCTCCACTGCTGTAGTAACCCGGGTCCGGGGCCTCGGTGGTGCTCC
TGATGCCCCTCACCCACCCCTGAAGATCCCAGGTGGGCGAGGGAATAGTCAGAGGG
ATCACAATCTTTCAGCTAACTTATTCTACTCCGATGATCGGCTGAATGTAACAGAGG
AACTAACGTCCAACGACAAGACGAGGATTCTCAACGTCCAGTCCAGGCTCACAGAC
GCCAAACGCATTAACTGGCGAACAGTGCTGAGTGGCGGCAGCCTCTACATCGAGAT
CCCGGGCGGCGCGCTGCCCGAGGGGAGCAAGGACAGCTTTGCAGTTCTCCTGGAGT
TCGCTGAGGAGCAGCTGCGAGCCGACCATGTCTTCATTTGCTTCCACAAGAACCGCG
AGGACAGAGCCGCCTTGCTCCGAACCTTCAGCTTTTTGGGCTTTGAGATTGTGAGAC
CGGGGCATCCCCTTGTCCCCAAGAGACCCGACGCTTGCTTCATGGCCTACACGTTCG
AGAGAGAGTCTTCGGGAGAGGAGGAGGAGTAGGGCCGCCTCGGGGCTGGGCATCC
GGCCCCTGGGGCCACCCCTTGTCAGCCGGGTGGGTAGGAACCGTAGACTCGCTCATC
TCGCCTGGGTTTGTCCGCATGTTGTAATCGTGCAAATAAACGCTCACTCCGAATTAG
CGGTGTATTTCTTGAAGTTTAATATTGTGTTTGTGATACTGAAGTATTTGCTTTAATT
CTAAATAAAAATTTATATTTTACTTTTTTATTGCTGGTTTAAGATGATTCAGATTATC
CTTGTACTTTGAGGAGAAGTTTCTTATTTGGAGTCTTTTGGAAACAGTCTTAGTCTTT
TAACTTGGAAAGATGAGGTATTAATCCCCTCCATTGCTCTCCAAAAGCCAATAAAGT
GATTACACCCGA
PNOC (SEQ ID NO: 25; NM_006228).
GCCAGGAAGGCTTGCAGGTTCTGCTGTTTGGTTGCTGAAGGGGGTCAGTGTGTGTAT
GTGTCATGGAGGTGGGCAGGGAAGGGGAGGGCTGTGCGTGGGGGAGAGGATATAT
ATGCTGGTGTGGCTGAGAAAGCGGAACCGAGCCTCGCATCCATCGGAGGGAGCCGG
GGACTGACAGCTCTCAGCACCTGCTTCCTGCTCCTGCACCATGAAAGTCCTGCTTTG
TGACCTGCTGCTGCTCAGTCTCTTCTCCAGTGTGTTCAGCAGTTGTCAGAGGGACTGT
CTCACATGCCAGGAGAAGCTCCACCCAGCCCTGGACAGCTTCGACCTGGAGGTGTG
CATCCTCGAGTGTGAAGAGAAGGTCTTCCCCAGCCCCCTCTGGACTCCATGCACCAA
GGTCATGGCCAGGAGCTCTTGGCAGCTCAGCCCTGCCGCCCCAGAGCATGTGGCGG
CTGCTCTCTACCAGCCGAGAGCTTCGGAGATGCAGCATCTGCGGCGAATGCCCCGA
GTCCGGAGCTTGTTCCAGGAGCAGGAAGAGCCCGAGCCTGGCATGGAGGAGGCTGG
TGAGATGGAGCAGAAGCAGCTGCAGAAGAGATTTGGGGGCTTCACCGGGGCCCGGA
AGTCGGCCAGGAAGTTGGCCAATCAGAAGCGGTTCAGTGAGTTTATGAGGCAATAC
TTGGTCCTGAGCATGCAGTCCAGCCAGCGCCGGCGCACCCTGCACCAGAATGGTAA
TGTGTAGCCGGAAGGGGCGCTCCTCCCAGCTGTACCGGCCACTGCAACCCATGAGC
GTCCAGGTGATCCCCCAAACAGCATGTGCTCAGCCCCAGACCTGCCGCCTGGGAATC
AGGATTCCTTCTTCCCCAAGGCACTGAGCGCCTGCAGATCCCGCAGGCTTCGTTTGC
CTCCAGAACCTTCCCGTCTGATTGTTCCTCCCCAGCCCCCTGGCATGTTTCACCACAA
CCCTGTTGCTACATCAGAGTGTATTTTTGTAATTCCTCTAGCTACCATTTCAATAGCC
CCATCTCTCCTGCTCACCCGCCTCTTGCCCCTTCTAGGGGCAGGTGAAAGGAATAGG
AAATTGAACCTGGGGTTTTGACTTGCCACTGCCATAACTTGTTTGTAAAAGAGCTGT
TCTTTTTGACTGATTGTTTTAAACAACGATTTCTCCATTAAACTTCTACTGAGCAAAT
GGTTAATAAAAAAAAAAAAAAAAAA
PDE4B (SEQ ID NO: 26; NM_002600).
AGAGCGCTGCGGCCGCGGCGGTGCAGCAGAGGCGCCTCGGGCAGGAGGAGGGCGG
CTTCTGCGAGGGCAGCCTGAGGTATTAAAAAGTGTCAGCAAACTGCATTGAATAAC
AGACATCCTAAGAGGGGATATTTTCCACCTCTATAATGAAGAAAAGCAGGAGTGTG
ATGACGGTGATGGCTGATGATAATGTTAAAGATTATTTTGAATGTAGCTTGAGTAAA
TCCTACAGTTCTTCCAGTAACACACTTGGGATCGACCTCTGGAGAGGGAGAAGGTGT
TGCTCAGGAAACTTACAGTTACCACCACTGTCTCAAAGACAGAGTGAAAGGGCAAG
GACTCCTGAGGGAGATGGTATTTCCAGGCCGACCACACTGCCTTTGACAACGCTTCC
AAGCATTGCTATTACAACTGTAAGCCAGGAGTGCTTTGATGTGGAAAATGGCCCTTC
CCCAGGTCGGAGTCCACTGGATCCCCAGGCCAGCTCTTCCGCTGGGCTGGTACTTCA
CGCCACCTTTCCTGGGCACAGCCAGCGCAGAGAGTCATTTCTCTACAGATCAGACAG
CGACTATGACTTGTCACCAAAGGCGATGTCGAGAAACTCTTCTCTTCCAAGCGAGCA
ACACGGCGATGACTTGATTGTAACTCCTTTTGCCCAGGTCCTTGCCAGCTTGCGAAG
TGTGAGAAACAACTTCACTATACTGACAAACCTTCATGGTACATCTAACAAGAGGTC
CCCAGCTGCTAGTCAGCCTCCTGTCTCCAGAGTCAACCCACAAGAAGAATCTTATCA
AAAATTAGCAATGGAAACGCTGGAGGAATTAGACTGGTGTTTAGACCAGCTAGAGA
CCATACAGACCTACCGGTCTGTCAGTGAGATGGCTTCTAACAAGTTCAAAAGAATGC
TGAACCGGGAGCTGACACACCTCTCAGAGATGAGCCGATCAGGGAACCAGGTGTCT
GAATACATTTCAAATACTTTCTTAGACAAGCAGAATGATGTGGAGATCCCATCTCCT
ACCCAGAAAGACAGGGAGAAAAAGAAAAAGCAGCAGCTCATGACCCAGATAAGTG
GAGTGAAGAAATTAATGCATAGTTCAAGCCTAAACAATACAAGCATCTCACGCTTTG
GAGTCAACACTGAAAATGAAGATCACCTGGCCAAGGAGCTGGAAGACCTGAACAAA
TGGGGTCTTAACATCTTTAATGTGGCTGGATATTCTCACAATAGACCCCTAACATGC
ATCATGTATGCTATATTCCAGGAAAGAGACCTCCTAAAGACATTCAGAATCTCATCT
GACACATTTATAACCTACATGATGACTTTAGAAGACCATTACCATTCTGACGTGGCA
TATCACAACAGCCTGCACGCTGCTGATGTAGCCCAGTCGACCCATGTTCTCCTTTCT
ACACCAGCATTAGACGCTGTCTTCACAGATTTGGAGATCCTGGCTGCCATTTTTGCA
GCTGCCATCCATGACGTTGATCATCCTGGAGTCTCCAATCAGTTTCTCATCAACACA
AATTCAGAACTTGCTTTGATGTATAATGATGAATCTGTGTTGGAAAATCATCACCTT
GCTGTGGGTTTCAAACTGCTGCAAGAAGAACACTGTGACATCTTCATGAATCTCACC
AAGAAGCAGCGTCAGACACTCAGGAAGATGGTTATTGACATGGTGTTAGCAACTGA
TATGTCTAAACATATGAGCCTGCTGGCAGACCTGAAGACAATGGTAGAAACGAAGA
AAGTTACAAGTTCAGGCGTTCTTCTCCTAGACAACTATACCGATCGCATTCAGGTCC
TTCGCAACATGGTACACTGTGCAGACCTGAGCAACCCCACCAAGTCCTTGGAATTGT
ATCGGCAATGGACAGACCGCATCATGGAGGAATTTTTCCAGCAGGGAGACAAAGAG
CGGGAGAGGGGAATGGAAATTAGCCCAATGTGTGATAAACACACAGCTTCTGTGGA
AAAATCCCAGGTTGGTTTCATCGACTACATTGTCCATCCATTGTGGGAGACATGGGC
AGATTTGGTACAGCCTGATGCTCAGGACATTCTCGATACCTTAGAAGATAACAGGAA
CTGGTATCAGAGCATGATACCTCAAAGTCCCTCACCACCACTGGACGAGCAGAACA
GGGACTGCCAGGGTCTGATGGAGAAGTTTCAGTTTGAACTGACTCTCGATGAGGAA
GATTCTGAAGGACCTGAGAAGGAGGGAGAGGGACACAGCTATTTCAGCAGCACAAA
GACGCTTTGTGTGATTGATCCAGAAAACAGAGATTCCCTGGGAGAGACTGACATAG
ACATTGCAACAGAAGACAAGTCCCCCGTGGATACATAATCCCCCTCTCCCTGTGGAG
ATGAACATTCTATCCTTGATGAGCATGCCAGCTATGTGGTAGGGCCAGCCCACCATG
GGGGCCAAGACCTGCACAGGACAAGGGCCACCTGGCCTTTCAGTTACTTGAGTTTGG
AGTCAGAAAGCAAGACCAGGAAGCAAATAGCAGCTCAGGAAATCCCACGGTTGACT
TGCCTTGATGGCAAGCTTGGTGGAGAGGGCTGAAGCTGTTGCTGGGGGCCGATTCTG
ATCAAGACACATGGCTTGAAAATGGAAGACACAAAACTGAGAGATCATTCTGCACT
AAGTTTCGGGAACTTATCCCCGACAGTGACTGAACTCACTGACTAATAACTTCATTT
ATGAATCTTCTCACTTGTCCCTTTGTCTGCCAACCTGTGTGCCTTTTTTGTAAAACATT
TTCATGTCTTTAAAATGCCTGTTGAATACCTGGAGTTTAGTATCAACTTCTACACAGA
TAAGCTTTCAAAGTTGACAAACTTTTTTGACTCTTTCTGGAAAAGGGAAAGAAAATA
GTCTTCCTTCTTTCTTGGGCAATATCCTTCACTTTACTACAGTTACTTTTGCAAACAG
ACAGAAAGGATACACTTCTAACCACATTTTACTTCCTTCCCCTGTTGTCCAGTCCAAC
TCCACAGTCACTCTTAAAACTTCTCTCTGTTTGCCTGCCTCCAACAGTACTTTTAACT
TTTTGCTGTAAACAGAATAAAATTGAACAAATTAGGGGGTAGAAAGGAGCAGTGGT
GTCGTTCACCGTGAGAGTCTGCATAGAACTCAGCAGTGTGCCCTGCTGTGTCTTGGA
CCCTGCCCCCCACAGGAGTTGTACAGTCCCTGGCCCTGTTCCCTACCTCCTCTCTTCA
CCCCGTTAGGCTGTTTTCAATGTAATGCTGCCGTCCTTCTCTTGCACTGCCTTCTGCG
CTAACACCTCCATTCCTGTTTATAACCGTGTATTTATTACTTAATGTATATAATGTAA
TGTTTTGTAAGTTATTAATTTATATATCTAACATTGCCTGCCAATGGTGGTGTTAAAT
TTGTGTAGAAAACTCTGCCTAAGAGTTACGACTTTTTCTTGTAATGTTTTGTATTGTG
TATTATATAACCCAAACGTCACTTAGTAGAGACATATGGCCCCCTTGGCAGAGAGGA
CAGGGGTGGGCTTTTGTTCAAAGGGTCTGCCCTTTCCCTGCCTGAGTTGCTACTTCTG
CACAACCCCTTTATGAACCAGTTTTGGAAACAATATTCTCACATTAGATACTAAATG
GTTTATACTGAGCTTTTACTTTTGTATAGCTTGATAGGGGCAGGGGGCAATGGGATG
TAGTTTTTACCCAGGTTCTATCCAAATCTATGTGGGCATGAGTTGGGTTATAACTGG
ATCCTACTATCATTGTGGCTTTGGTTCAAAAGGAAACACTACATTTGCTCACAGATG
ATTCTTCTGAATGCTCCCGAACTACTGACTTTGAAGAGGTAGCCTCCTGCCTGCCATT
AAGCAGGAATGTCATGTTCCAGTTCATTACAAAAGAAAACAATAAAACAATGTGAA
TTTTTATAATAAAATGTGAACTGATGTAGCAAATTACGCAAATGTGAAGCCTCTTCT
GATAACACTTGTTAGGCCTCTTACTGATGTCAGTTTCAGTTTGTAAAATATGTTTCAT
GCTTTCAGTTCAGCATTGTGACTCAGTAATTACAGAAAATGGCACAAATGTGCATGA
CCAATGTATGTCTATGAACACTGCATTGTTTCAGGTGGACATTTTATCATTTTCAAAT
GTTTCTCACAATGTATGTTATAGTATTATTATTATATATTGTGTTCAAATGCATTCTA
AAGAGACTTTTATATGAGGTGAATAAAGAAAAGCATGATTAGATTAAAAAAA
SCARB1 (SEQ ID NO: 27; NM_005505.4).
GCTCAGGCCCCGCCCCTGCCGCCGGAATCCTGAAGCCCAAGGCTGCCCGGGGGCGG
TCCGGCGGCGCCGGCGATGGGGCATAAAACCACTGGCCACCTGCCGGGCTGCTCCT
GCGTGCGCTGCCGTCCCGGATCCACCGTGCCTCTGCGGCCTGCGTGCCCGGAGTCCC
CGCCTGTGTCGTCTCTGTCGCCGTCCCCGTCTCCTGCCAGGCGCGGAGCCCTGCGAG
CCGCGGGTGGGCCCCAGGCGCGCAGACATGGGCTGCTCCGCCAAAGCGCGCTGGGC
TGCCGGGGCGCTGGGCGTCGCGGGGCTACTGTGCGCTGTGCTGGGCGCTGTCATGAT
CGTGATGGTGCCGTCGCTCATCAAGCAGCAGGTCCTTAAGAACGTGCGCATCGACCC
CAGTAGCCTGTCCTTCAACATGTGGAAGGAGATCCCTATCCCCTTCTATCTCTCCGTC
TACTTCTTTGACGTCATGAACCCCAGCGAGATCCTGAAGGGCGAGAAGCCGCAGGT
GCGGGAGCGCGGGCCCTACGTGTACAGGGAGTTCAGGCACAAAAGCAACATCACCT
TCAACAACAACGACACCGTGTCCTTCCTCGAGTACCGCACCTTCCAGTTCCAGCCCT
CCAAGTCCCACGGCTCGGAGAGCGACTACATCGTCATGCCCAACATCCTGGTCTTGG
GTGCGGCGGTGATGATGGAGAATAAGCCCATGACCCTGAAGCTCATCATGACCTTG
GCATTCACCACCCTCGGCGAACGTGCCTTCATGAACCGCACTGTGGGTGAGATCATG
TGGGGCTACAAGGACCCCCTTGTGAATCTCATCAACAAGTACTTTCCAGGCATGTTC
CCCTTCAAGGACAAGTTCGGATTATTTGCTGAGCTCAACAACTCCGACTCTGGGCTC
TTCACGGTGTTCACGGGGGTCCAGAACATCAGCAGGATCCACCTCGTGGACAAGTG
GAACGGGCTGAGCAAGGTTGACTTCTGGCATTCCGATCAGTGCAACATGATCAATG
GAACTTCTGGGCAAATGTGGCCGCCCTTCATGACTCCTGAGTCCTCGCTGGAGTTCT
ACAGCCCGGAGGCCTGCCGATCCATGAAGCTAATGTACAAGGAGTCAGGGGTGTTT
GAAGGCATCCCCACCTATCGCTTCGTGGCTCCCAAAACCCTGTTTGCCAACGGGTCC
ATCTACCCACCCAACGAAGGCTTCTGCCCGTGCCTGGAGTCTGGAATTCAGAACGTC
AGCACCTGCAGGTTCAGTGCCCCCTTGTTTCTCTCCCATCCTCACTTCCTCAACGCTG
ACCCGGTTCTGGCAGAAGCGGTGACTGGCCTGCACCCTAACCAGGAGGCACACTCC
TTGTTCCTGGACATCCACCCGGTCACGGGAATCCCCATGAACTGCTCTGTGAAACTG
CAGCTGAGCCTCTACATGAAATCTGTCGCAGGCATTGGACAAACTGGGAAGATTGA
GCCTGTGGTCCTGCCGCTGCTCTGGTTTGCAGAGAGCGGGGCCATGGAGGGGGAGA
CTCTTCACACATTCTACACTCAGCTGGTGTTGATGCCCAAGGTGATGCACTATGCCC
AGTACGTCCTCCTGGCGCTGGGCTGCGTCCTGCTGCTGGTCCCTGTCATCTGCCAAA
TCCGGAGCCAAGAGAAATGCTATTTATTTTGGAGTAGTAGTAAAAAGGGCTCAAAG
GATAAGGAGGCCATTCAGGCCTATTCTGAATCCCTGATGACATCAGCTCCCAAGGGC
TCTGTGCTGCAGGAAGCAAAACTGTAGGGTCCTGAGGACACCGTGAGCCAGCCAGG
CCTGGCCGCTGGGCCTGACCGGCCCCCCAGCCCCTACACCCCGCTTCTCCCGGACTC
TCCCAGCGGACAGCCCCCCAGCCCCACAGCCTGAGCCTCCCAGCTGCCATGTGCCTG
TTGCACACCTGCACACACGCCCTGGCACACATACACACATGCGTGCAGGCTTGTGCA
GACACTCAGGGATGGAGCTGCTGCTGAAGGGACTTGTAGGGAGAGGCTCGTCAACA
AGCACTGTTCTGGAACCTTCTCTCCACGTGGCCCACAGGCCTGACCACAGGGGCTGT
GGGTCCTGCGTCCCCTTCCTCGGGTGAGCCTGGCCTGTCCCGTTCAGCCGTTGGGCC
CAGGCTTCCTCCCCTCCAAGGTGAAACACTGCAGTCCCGGTGTGGTGGCTCCCCATG
CAGGACGGGCCAGGCTGGGAGTGCCGCCTTCCTGTGCCAAATTCAGTGGGGACTCA
GTGCCCAGGCCCTGGCCACGAGCTTTGGCCTTGGTCTACCTGCCAGGCCAGGCAAAG
CGCCTTTACACAGGCCTCGGAAAACAATGGAGTGAGCACAAGATGCCCTGTGCAGC
TGCCCGAGGGTCTCCGCCCACCCCGGCCGGACTTTGATCCCCCCGAAGTCTTCACAG
GCACTGCATCGGGTTGTCTGGCGCCCTTTTCCTCCAGCCTAAACTGACATCATCCTAT
GGACTGAGCCGGCCACTCTCTGGCCGAAGTGGCCGCAGGCTGTGCCCCCGAGCTGC
CCCCACCCCCTCACAGGGTCCCTCAGATTATAGGTGCCCAGGCTGAGGTGAAGAGG
CCTGGGGGCCCTGCCTTCCGGGCGCTCCTGGACCCTGGGGCAAACCTGTGACCCTTT
TCTACTGGAATAGAAATGAGTTTTATCATCTTTGAAAAATAATTCACTCTTGAAGTA
ATAAACGTTTAAAAAAATGGGAAAAAAAAAAAAAAAAAA
TMEM9B (SEQ ID NO: 28; NM_020644.2).
GTGCGCGAACGGCTCCGGCCCGCACGGGTCGCCAGAGGCGACTGTGTGACACTCGG
AGTTTGCTGGGGTCTCCGTGGGCGGGAGGACTTTCCAGCGCAATGGCGACTCCCTAA
GCCCCGCAGCTTCTGCGCCCGGGAAAGATATCCAAGAGATGCAAAGCTCTACTGGG
CCCAGGCTGCCACCCCAGAGGCCCCCTTCCGTCCCGGGGCCGGGGCTAGGCCAAGG
CGGGCACCAGGACTGCCCAGCCTCCCGGCCCTTCGCACTGGTAACCGGTTCCGGGGC
GGATGCTTTTTGCATCTGACCCGGCGCGCCCGGTGACGCCTTCGCGTCCAGACGGAA
GTGCGGGCGGAGGATCCCCAGCCGGGTCCCAAGCCTGTGCCTGAGCCTGAGCCTGA
GCCTGAGCCCGAGCCGGGAGCCGGTCGCGGGGGCTCCGGGCTGTGGGACCGCTGGG
CCCCCAGCGATGGCGACCCTGTGGGGAGGCCTTCTTCGGCTTGGCTCCTTGCTCAGC
CTGTCGTGCCTGGCGCTTTCCGTGCTGCTGCTGGCGCAGCTGTCAGACGCCGCCAAG
AATTTCGAGGATGTCAGATGTAAATGTATCTGCCCTCCCTATAAAGAAAATTCTGGG
CATATTTATAATAAGAACATATCTCAGAAAGATTGTGATTGCCTTCATGTTGTGGAG
CCCATGCCTGTGCGGGGGCCTGATGTAGAAGCATACTGTCTACGCTGTGAATGCAAA
TATGAAGAAAGAAGCTCTGTCACAATCAAGGTTACCATTATAATTTATCTCTCCATT
TTGGGCCTTCTACTTCTGTACATGGTATATCTTACTCTGGTTGAGCCCATACTGAAGA
GGCGCCTCTTTGGACATGCACAGTTGATACAGAGTGATGATGATATTGGGGATCACC
AGCCTTTTGCAAATGCACACGATGTGCTAGCCCGCTCCCGCAGTCGAGCCAACGTGC
TGAACAAGGTAGAATATGCACAGCAGCGCTGGAAGCTTCAAGTCCAAGAGCAGCGA
AAGTCTGTCTTTGACCGGCATGTTGTCCTCAGCTAATTGGGAATTGAATTCAAGGTG
ACTAGAAAGAAACAGGCAGACAACTGGAAAGAACTGACTGGGTTTTGCTGGGTTTC
ATTTTAATACCTTGTTGATTTCACCAACTGTTGCTGGAAGATTCAAAACTGGAAGCA
AAAACTTGCTTGATTTTTTTTTCTTGTTAACGTAATAATAGAGACATTTTTAAAAGCA
CACAGCTCAAAGTCAGCCAATAAGTCTTTTCCTATTTGTGACTTTTACTAATAAAAAT
AAATCTGCCTGTAAATTATCTTGAAGTCCTTTACCTGGAACAAGCACTCTCTTTTTCA
CCACATAGTTTTAACTTGACTTTCAAGATAATTTTCAGGGTTTTTGTTGTTGTTGTTTT
TTGTTTGTTTGTTTTGGTGGGAGAGGGGAGGGATGCCTGGGAAGTGGTTAACAACTT
TTTTCAAGTCACTTTACTAAACAAACTTTTGTAAATAGACCTTACCTTCTATTTTCGA
GTTTCATTTATATTTTGCAGTGTAGCCAGCCTCATCAAAGAGCTGACTTACTCATTTG
ACTTTTGCACTGACTGTATTATCTGGGTATCTGCTGTGTCTGCACTTCATGGTAAACG
GGATCTAAAATGCCTGGTGGCTTTTCACAAAAAGCAGATTTTCTTCATGTACTGTGA
TGTCTGATGCAATGCATCCTAGAACAAACTGGCCATTTGCTAGTTTACTCTAAAGAC
TAAACATAGTCTTGGTGTGTGTGGTCTTACTCATCTTCTAGTACCTTTAAGGACAAAT
CCTAAGGACTTGGACACTTGCAATAAAGAAATTTTATTTTAAACCCAAGCCTCCCTG
GATTGATAATATATACACATTTGTCAGCATTTCCGGTCGTGGTGAGAGGCAGCTGTT
TGAGCTCCAATGTGTGCAGCTTTGAACTAGGGCTGGGGTTGTGGGTGCCTCTTCTGA
AAGGTCTAACCATTATTGGATAACTGGCTTTTTTCTTCCTATGTCCTCTTTGGAATGT
AACAATAAAAATAATTTTTGAAACATCCATCAGTGTATCTATCTATGTCTCCTAGTTT
TTTCCTCCTCCCTCTTTTGCTGTATAATGAGATTGAAGATATAAAGACATTTTGTACC
CTGTAAAAAAAA
PPP6R3 (SEQ ID NO: 29; XM_005274081).
AACTCAAGGCCTGCTTGATACGTCCGCCATTTTGGGCGCTTCGCTGATGGTGTCGGT
GAGCGCGTTTCCCGCCTGAGCGCAACTAGCGGCGGGTCGTGGGCACCTCCAGGAGA
GCTTGTTTCATATCCATATCCCACTGTATTCCTGCTAATCTGCTAATGCAGTAAATTG
GAGGAAAACTGTTACCAGGATAACCTGTAATGGGCAAGGAGCCACAAAGAAGAAA
ACATTTCTTTTAATTTTTAAACTTGGTTTGAAAGACCAGCATGTTTTGGAAATTTGAT
CTTCACTCATCATCCCACATAGACACACTTCTAGAAAGAGAAGATGTAACACTGAAG
GAGTTAATGGATGAGGAAGATGTTTTACAGGAATGTAAAGCTCAGAACCGCAAACT
TATAGAGTTTCTGTTAAAAGCAGAATGTCTCGAAGATTTAGTCTCATTCATTATAGA
AGAACCACCTCAAGACATGGATGAAAAGATCAGATACAAGTATCCAAATATATCTT
GTGAGTTGCTCACTTCTGATGTCTCCCAGATGAATGATAGACTGGGAGAAGATGAAT
CCTTGCTAATGAAATTATATAGCTTCCTCCTAAACGATTCCCCTTTGAATCCACTACT
TGCCAGTTTCTTCAGCAAGGTGCTAAGTATTCTTATCAGCAGAAAACCAGAACAGAT
TGTGGATTTCTTAAAGAAGAAGCATGATTTTGTAGACCTTATTATAAAGCACATAGG
AACTTCTGCTATCATGGATTTGTTGCTCAGGCTCCTGACGTGTATCGAACCTCCACAG
CCCAGGCAAGATGTGCTGAATTGGTTAAATGAGGAGAAAATTATCCAGAGGCTTGT
GGAAATAGTTCATCCATCGCAAGAAGAAGATCGACATTCAAATGCATCACAATCAC
TTTGTGAAATTGTTCGCCTGAGCAGAGACCAGATGTTACAAATTCAGAACAGTACAG
AGCCCGACCCCCTGCTTGCCACTCTAGAAAAGCAAGAAATTATAGAGCAGCTTCTAT
CAAATATTTTCCACAAGGAGAAAAATGAGTCAGCCATAGTCAGTGCAATCCAGATA
TTGCTGACTTTACTTGAGACACGACGACCAACATTTGAAGGCCATATAGAGATCTGC
CCACCAGGCATGAGCCATTCAGCTTGTTCAGTAAACAAGAGTGTTCTAGAAGCCATC
AGAGGAAGACTTGGATCTTTTCATGAACTCCTGCTGGAGCCACCCAAGAAAAGTGT
GATGAAGACCACATGGGGTGTGCTGGATCCTCCTGTGGGGAATACCCGGTTGAATGT
CATTAGGTTGATATCCAGCCTGCTTCAAACCAATACCAGCAGTATAAATGGGGACCT
TATGGAGCTGAATAGCATTGGAGTCATATTGAACATGTTCTTCAAGTATACATGGAA
TAACTTTTTGCATACACAAGTGGAAATTTGTATTGCACTGATTCTTGCAAGTCCTTTT
GAAAACACAGAAAATGCCACAATTACCGATCAAGACTCCACTGGTGATAATTTGTT
ATTAAAACATCTTTTCCAAAAATGTCAATTAATAGAACGAATACTTGAAGCCTGGGA
AATGAATGAGAAGAAACAGGCTGAGGGAGGAAGACGGCATGGTTACATGGGACAC
CTAACGAGGATAGCTAACTGTATCGTGCACAGCACTGACAAGGGCCCCAACAGTGC
ATTAGTGCAGCAGCTTATCAAAGATCTTCCCGACGAAGTCAGGGAACGATGGGAGA
CGTTCTGCACAAGCTCCTTAGGAGAAACTAACAAGAGGAACACGGTAGATCTAGTT
ACAACCTGCCATATTCATTCATCCAGTGATGATGAAATTGACTTTAAAGAAACGGGT
TTCTCACAGGATTCTTCTTTGCAGCAAGCCTTTTCTGATTATCAGATGCAACAAATGA
CGTCCAATTTTATTGACCAGTTTGGCTTCAACGATGAGAAGTTTGCAGATCAAGATG
ACATTGGCAATGTTTCTTTTGATCGAGTATCAGACATCAACTTTACTCTCAATACAAA
TGAAAGTGGAAATATTGCCTTGTTTGAAGCATGTTGTAAGGAAAGAATACAACAGTT
TGATGATGGTGGCTCTGATGAGGAAGATATATGGGAGGAAAAGCACATCGCATTCA
CACCAGAATCCCAAAGACGATCCAGCTCGGGGAGTACAGACAGTGAGGAAAGTAC
AGACTCTGAAGAAGAAGATGGAGCAAAGCAAGACTTGTTTGAACCCAGCAGTGCCA
ACACGGAGGATAAAATGGAGGTGGACCTGAGTGAACCACCCAACTGGTCAGCTAAC
TTTGATGTCCCAATGGAAACAACCCACGGTGCTCCATTGGATTCTGTGGGATCTGAT
GTCTGGAGCACAGAGGAGCCGATGCCAACTAAAGAGACGGGCTGGGCTTCTTTTTC
AGAGTTCACGTCTTCCCTGAGCACAAAAGATTCTTTAAGGAGTAATTCTCCAGTGGA
AATGGAAACCAGCACTGAACCCATGGACCCTCTGACTCCCAGTGCGGCTGCCCTGG
CAGTGCAGCCAGAAGCGGCAGGCAGTGTGGCCATGGAAGCCAGCTCTGACGGAGA
GGAGGATGCAGAAAGTACAGACAAGGTAACTGAGACAGTGATGAATGGCGGCATG
AAGGAAACGCTCAGCCTCACTGTAGATGCCAAGACAGAGACTGCGGTCTTCAAAAG
TGAGGAAGGGAAACTGTCTACCTCTCAAGATGCTGCTTGTAAAGACGCAGAGGAGT
GTCCCGAGACTGCAGAGGCGAAGTGCGCGGCGCCCAGGCCTCCCAGCAGCAGTCCC
GAGCAGAGTGCCTCCGATGCCTGTCTGTTGCTCCTTAGGACTGGCCAACCAAGCGCA
CCAGGTGACACTTCAGTGAATGGCCCTGTATGACGGGTGACGTCTGCTGCTGCTGAC
TGAGGACTGCAGACCGCCACCACTCAGGGGCTCTGGAGGGGTCAGCTGGAGCCCAC
CAAGCTGTCACTGCTGCACTCACTCTGCAAGGGATCAGGACCAGCAACCTTTATATT
CTAGATTCTAAGACATTGTACAGAGAAATTCAGAAGTGTAAAAATATTGCACATTGA
CAAATACCAAGAATTTTTGCGTATGTTTATATTGTATTGTTCTAAATAATGGGTAGCC
TGTGAAATAAGATCTTGCCACCCATGTAATAATAGTAGTAATACTATAGTTAAAATG
GCTGTAAGAATAGTTTTATAAAAGTGAATACACAGATCTATTGTATTTGAAACATAA
CTTTGACAATTATTAGTGTGACCAAAGTATTAGGCGGTTTTCATACATTTTTCACCTT
GTACAAAATTATGAATTCATTTTTCCTCCAGGCCGACAAGGAGTTGTAGAATGAAAA
TGCCCTCTAAGTGTTATTTTGGTTGTTCTAACTTACAAAAGTGATTTTGAATAAGAAA
TATTTGGTGTTCTTTTTATAACCAGTTTTTGATTGGTAATTGTTTTCTGTATTGTTTAA
AACGGATCAAAAATGTAAGTCTATTGGTAGAGATTAAGTAAAGTATTTATTGCTACA
TCATAGTTGATAAATTGATGTTATCGTAAAGCCATATGTTCTGTTCAAGTCTTGTTTG
CTTGAAATGATTATTCCTACAAGTGAAACACTAGACTATTTGGAGTGTATATGGCTT
GTGTTTTGGGATTTTTTTTTTTTTTTTTTGGCTTTTGTTTTTGTTTGTTTTTTTGTTTCAT
TTGGTAGTTCATCTGCCTTTTAACCCATTCACCAAAATTTACCTTGTTAACAAGCATC
ACCAATGAACATTTCAGAGCAATCTGCATATTTAACAGACCTAAAATAAATCCTATT
AGGCAAGTCAGTTGAAAATGCTCGTGCTGCTAATGGAATTAGAGTGCGTTCATTTTA
CAGGCTAGTATTTTAAAAGTAGAAATCAAAATCTGGCACCGAAGCATGCTAATTGTT
TACTGTACCTTGTGAGGTTTTCACTCATAAATTTAAACCAGTGTATTTTTTTAGAACT
GGTTTGTGTATATATATAGTGATTATGGATACTAATTCAATGTAATTTATAATTTTCT
ATGTCAATACAAAAATACATCACAGCCTTCTCAAACAGCTCAAGCAATATATTGTAT
ATTGCCATATCGTCTGGTGAAAGGGTTAAATTACTTCACCTCTTGCACTTTTAGATGC
AAATCAGTTTTTCATTTCTGTAATAGAAAATTATTCACGTATTTTTACATCATTTGTTT
TTCCTGACCAGTATTTAAAACCAAAAGGATATTCTGAAAAATGGCCAACAATTTTTT
TAGAAGTAGCATCCCAAGCAGCGTGCCTAAACATTACATTGCATATGGAAATAAAA
GAATCAAACGTCTAATGCCTTATTATTTCTGATTTCCTTTTTCATTTTAAGTGGTGTG
GAGATTCCAGCACTCCCAGGACAGTGGAGTCAGCAGTAAGCCCTGGGACAGGTGGC
AAGGGTGGGTCCCTTGACCTTTGCACGCCTCCTCAGGAACCCCCTTTCCCGGGTGAG
CCCCTCTCTGAAGAGACTGTCCTTGGGCCTCCTCTGGAAGCAGCACCCCCAGAGGAC
AGGGCTCCTCCTGCTTGCCTCAGGGCTGCCTGACTTGAATGGCGTTGGACCTCGGGG
ATTACTGGTAGATAATATGCTCTGGTCTCGCCTGGTGGTGAGTTTTGCCAGCCATGG
CCAGGGTTTGGCTCCACTGGTGGCACACGTGGCCTCCGTGGTATGGACCTGGTGGCT
TCTCCATCCCACTGTGGCCTCTGTGGTATGGACCTGGTGGCTTCTCCATCCTACCCAA
GGTAACAGTGTCTTGCTTCATCCCACTGACTGCTGGGAGAGAGCCTCTGGGACTTTT
CTTTGGGGCATCATTTTGTTTTGTCTTTCGTAGCAGGGAAAGGATATGACAATGGGG
AGGACAGTTCTTTTGGAGGTTGGAGGGGCCAAGCCAAGGACAGGAGCAAGTGTGCC
CTCATTTTGTTTCTACTTTTAATTTCTGTGTGTTGGCCATACTGAATTATGAGACTAA
CAGATGTCTACAATACAATACCTGTATTCAAAATAACAAAAATAAAGCCTGATTCTT
TGTTTCTAGAAA

1. A radiation biodosimetry assay system, comprising:
a set of radiation exposure positive control mRNA samples or cDNAs thereof, wherein the positive control mRNA samples or cDNAs thereof are based on or derived from a human subject having absorbed about 0.5 Grays or more of ionizing radiation, and
a plurality of in vitro nucleic acid amplification reaction mixtures, each amplification reaction mixture comprising:
(a) a probe of at least 10 nucleotides complementary to at least 10 consecutive nucleotides of at least one of SEQ ID NOs: 1-29, or the complementary sequences thereof, labeled with a fluorescent dye and a quencher; and
(b) one or more primer pairs, each primer pair comprising primers of at least 10 nucleotides, complementary to at least 10 consecutive nucleotides of at least one of SEQ ID NOs: 1-29, or a complementary sequence thereof,
wherein the system comprises probes of (a) and primer pairs of (b) complementary to at least 10 consecutive nucleotides of each of the following:
(i) a region of SEQ ID NO: 1 from position 2098-2271;
(ii) SEQ ID NO: 8;
(iii) SEQ ID NO: 9;
(iv) SEQ ID NO: 10; and
(v) SEQ ID NO: 14,
or the complementary sequences thereof, and wherein the system is configured to enable calculation of an estimated absorbed radiation dose based on a multi-gene regression model and a mathematical algorithm.

2. The radiation biodosimetry assay system of claim 1, wherein the system further comprises primers of (a) and probes of (b) complementary to at least 10 consecutive nucleotides of at least one of SEQ ID NOs: 2-7, 11-13, 15-29, or a complementary sequence thereof.

3. The radiation biodosimetry assay system of claim 1, wherein the in vitro nucleic acid amplification reaction mixtures are provided in a multi-well plate.

4. The radiation biodosimetry assay system of claim 3, wherein at least two nucleic acid probes complementary to at least two different nucleic acid targets are in the same wells of the multi-well plate.

5. A radiation biomarker assay kit, comprising:
a nucleic acid probe set comprising a plurality of nucleic acid probes of at least 10nucleotides each that are complementary to at least 10 consecutive nucleotides to at least three of SEQ ID NOs: 1-29 or the complementary sequences thereof, the nucleic acid probe set further comprising a probe detectably labeled with a fluorescent dye and a quencher and configured for PCR amplification,
a set of primer pairs comprising a plurality of primer pairs each comprising primers of at least 10 nucleotides that are complementary to at least 10 consecutive nucleotides to at least three of SEQ ID NOs: 1-29 or the complementary sequences thereof,
wherein at least one of the nucleic acid probes of the nucleic acid probe set or one of the primer pairs of the set of primer pairs are complementary to each of the following:
(a) a region of SEQ ID NO: 1 from position 2098-2271.
(b) SEQ ID NO: 8;
(c) SEQ ID NO: 9;
(d) SEQ ID NO: 10; and
(e) SEQ ID NO 14;
or the complementary sequences thereof,
a set of radiation exposure positive control mRNA samples or cDNAs thereof, wherein the positive control mRNA samples or cDNAs thereof are based on or derived from a human subject having absorbed about 0.5 Grays or more of ionizing radiation, and
instructions for calculating an estimate of absorbed radiation dose from a fluorescence value obtained by contacting in vitro an mRNA sample from a human subject suspected of suffering from radiation exposure or cDNA derived therefrom to the nucleic acid probe set and a thermostable polymerase under PCR conditions, wherein the estimate is calculated based on a multi-gene regression model and a mathematical algorithm.

6. The radiation biomarker assay kit of claim 5, wherein the nucleic acid probe set further comprises probes complementary to at least 10 consecutive nucleotides of at least one of SEQ ID NOs: 2-7, 11-13, 15-29, or a complementary sequence thereof.

7. The radiation biomarker assay kit of claim 5, further comprising radiation exposure negative control mRNA samples or cDNAs thereof.

8. The radiation biomarker assay kit of claim 5, wherein the nucleic acid probes are provided in a multi-well plate.

9. The radiation biomarker assay kit of claim 8, wherein at least two nucleic acid probes complementary to at least two different nucleic acid targets are in the same wells of the multi-well plate.

10. A method for estimating an absorbed dose of ionizing radiation received by a subject, comprising:
(i) determining mRNA expression levels of mRNAs comprising at least one of SEQ ID NOs: 1-39 in a stabilized blood sample comprising mRNA from the subject to obtain a gene expression profile; and
(ii) transforming the gene expression profile into an estimate of absorbed radiation dose for the subject based on a mathematical algorithm.

11. The method of claim 10, wherein transforming in step (ii) comprises calculating an estimate of absorbed radiation dose from a fluorescence value obtained by contacting in vitro an mRNA sample from a human subject suspected of suffering from radiation exposure or cDNA derived therefrom to a nucleic acid probe set and a thermostable polymerase under PCR conditions.

12. The method of claim 11, wherein the nucleic acid probe set comprises a plurality of nucleic acid probes complementary to at least 10 consecutive nucleotides of at least three of SEQ ID NOs:
1-39 or the complementary sequences thereof, the nucleic acid probe set further comprising a probe detectably labeled with a fluorescent dye and a quencher and configured for PCR amplification.

13. The method of claim 11, wherein the mRNA sample from a human subject or cDNA derived therefrom is further contacted to a set of primer pairs comprising a plurality of primer pairs each comprising primers complementary to at 10 consecutive nucleotides of at least three of SEQ ID NOs: 1-39 or the complementary sequences thereof.

14. A method for radiation treatment triage of a subject in need thereof comprising:
(i) determining mRNA expression levels of mRNAs comprising at least one of SEQ ID NOs: 1-39 in a stabilized blood sample comprising mRNA from the subject to obtain a gene expression profile; and
(ii) providing a suitable treatment for radiation exposure to the subject based on the gene expression profile.

15. The method of claim 14, further comprising transforming the gene expression profile into an estimate of an estimate of an absorbed radiation dose for the subject.

16. The method of claim 15, wherein transforming comprises calculating the estimate of the absorbed radiation dose from a fluorescence value obtained by contacting in vitro an mRNA sample from a human subject suspected of suffering from radiation exposure or cDNA derived therefrom to a nucleic acid probe set and a thermostable polymerase under PCR conditions suitable for amplification of the mRNA or cDNA.

17. The method of claim 16, wherein the nucleic acid probe set comprises a plurality of nucleic acid probes complementary to at least 10 consecutive nucleotides of at least three of SEQ ID NOs: 1-39 or the complementary sequences thereof, the nucleic acid probe set further comprising a probe detectably labeled with a fluorescent dye and a quencher and configured for PCR amplification.

18. The method of claim 17, wherein the mRNA sample from the human subject or CDNA derived therefrom is further contacted to a set of primer pairs comprising a plurality of primers each complementary to at least 10 consecutive nucleotides of at least three of SEQ ID NOs: 1-39 or the complementary sequences thereof.


SEQUENCE LISTING (31 sequences)

SEQ ID NO: 1 - DNA, 4184 bp, Homo sapiens
  source 1..4184: mol_type = genomic DNA
atttaagggcccgcctctcctggctcacagctgcttgctgctccagccttgccctcccagagctgccggacgctcgcgggtctcggaacgcatcccgccgcgggggcttcggccgtggcatgggcgccgcgggcctgctcggggttttcttggctctcgtcgcaccgggggtcctcgggatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacgctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtgaagaaattttttgcccatcacctccccctattctcaatggaagacatataggcaactcactagcaaatgtctcatatggaagcatagtcacttacacttgtgacccggacccagaggaaggagtgaacttcatccttattggagagagcactctccgttgtacagttgatagtcagaagactgggacctggagtggccctgccccacgctgtgaactttctacttctgcggttcagtgtccacatccccagatcctaagaggccgaatggtatctgggcagaaagatcgatatacctataacgacactgtgatatttgcttgcatgtttggcttcaccttgaagggcagcaagcaaatccgatgcaatgcccaaggcacatgggagccatctgcaccagtctgtgaaaaggaatgccaggcccctcctaacatcctcaatgggcaaaaggaagatagacacatggtccgctttgaccctggaacatctataaaatatagctgtaaccctggctatgtgctggtgggagaagaatccatacagtgtacctctgagggggtgtggacaccccctgtaccccaatgcaaagtggcagcgtgtgaagctacaggaaggcaactcttgacaaaaccccagcaccaatttgttagaccagatgtcaactcttcttgtggtgaagggtacaagttaagtgggagtgtttatcaggagtgtcaaggcacaattccttggtttatggagattcgtctttgtaaagaaatcacctgcccaccaccccctgttatctacaatggggcacacaccgggagttccttagaagattttccatatggaaccacggtcacttacacatgtaaccctgggccagaaagaggagtggaattcagcctcattggagagagcaccatccgttgtacaagcaatgatcaagaaagaggcacctggagtggccctgctcccctgtgtaaactttccctccttgctgtccagtgctcacatgtccatattgcaaatggatacaagatatctggcaaggaagccccatatttctacaatgacactgtgacattcaagtgttatagtggatttactttgaagggcagtagtcagattcgttgcaaagctgataacacctgggatcctgaaataccagtttgtgaaaaaggctgccagtcacctcctgggctccaccatggtcgtcatacaggtggaaatacggtcttctttgtctctgggatgactgtagactacacttgtgaccctggctatttgcttgtgggaaacaaatccattcactgtatgccttcaggaaattggagtccttctgccccacggtgtgaagaaacatgccagcatgtgagacagagtcttcaagaacttccagctggttcacgtgtggagctagttaatacgtcctgccaagatgggtaccagttgactggacatgcttatcagatgtgtcaagatgctgaaaatggaatttggttcaaaaagattccactttgtaaagttattcactgtcaccctccaccagtgattgtcaatgggaagcacacaggcatgatggcagaaaactttctatatggaaatgaagtctcttatgaatgtgaccaaggattctatctcctgggagagaaaaaattgcagtgcagaagtgattctaaaggacatggatcttggagcgggccttccccacagtgcttacgatctcctcctgtgactcgctgccctaatccagaagtcaaacatgggtacaagctcaataaaacacattctgcatattcccacaatgacatagtgtatgttgactgcaatcctggcttcatcatgaatggtagtcgcgtgattaggtgtcatactgataacacatgggtgccaggtgtgccaacttgtatcaaaaaagccttcatagggtgtccacctccgcctaagacccctaacgggaaccatactggtggaaacatagctcgattttctcctggaatgtcaatcctgtacagctgtgaccaaggctacctgctggtgggagaggcactccttctttgcacacatgagggaacctggagccaacctgcccctcattgtaaagaggtaaactgtagctcaccagcagatatggatggaatccagaaagggctggaaccaaggaaaatgtatcagtatggagctgttgtaactctggagtgtgaagatgggtatatgctggaaggcagtccccagagccagtgccaatcggatcaccaatggaaccctcccctggcggtttgcagatcccgttcacttgctcctgtcctttgtggtattgctgcaggtttgatacttcttaccttcttgattgtcattaccttatacgtgatatcaaaacacagagcacgcaattattatacagatacaagccagaaagaagcttttcatttagaagcacgagaagtatattctgttgatccatacaacccagccagctgatcagaagacaaactggtgtgtgcctcattgcttggaattcagcggaatattgattagaaagaaactgctctaatatcagcaagtctctttatatggcctcaagatcaatgaaatgatgtcataagcgatcacttcctatatgcacttattctcaagaagaacatctttatggtaaagatgggagcccagtttcactgccatatactcttcaaggactttctgaagcctcacttatgagatgcctgaagccaggccatggctataaacaattacatggctctaaaaagttttgccctttttaaggaaggcactaaaaagagctgtcctggtatctagacccatcttctttttgaaatcagcatactcaatgttactatctgcttttggttataatgtgtttttaattatctaaagtatgaagcattttctggggttatgatggctttacctttattaggaagtatggttttattttgatagtagcttcctcctctggtggtgttaatcatttcatttttacccttacttggtttgagtttctctcacattactgtatatactttgcctttccataatcactcagtgattgcaatttgcacaagtttttttaaattatgggaatcaagatttaatcctagagatttggtgtacaattcaggctttggatgtttctttagcagttttgtgataagttctagttgcttgtaaaatttcacttaataatgtgtacattagtcattcaataaattgtaattgtaaagaaaacatacaaaaaaaaaaaaaaaa

SEQ ID NO: 2 - DNA, 1442 bp, Homo sapiens
  source 1..1442: mol_type = genomic DNA
agtcgggcagctctccgggccggcgtgggagcccgcgctccaaagcccggtggggggaggggcgctcacgcaaccgccactgtctggagcgggctcgcctctgcggcggcactcaccgcccgggctttactgaagcggagtctagcatgtgcggctgctccacagcggtgtgggtggcggcggctcctctgcagcagcctcggcagtaggggtcacggtggccaagcccaccgtggagctcatctgagagttgtaaggtacgggactgcctcggtctttgggacgccccgtctggtagcatcccagatccagcacgttccttccggccctgcaccccggcccggtgcctcacaccccgctaccccatgcatccagactctaaggcagcccctgcatctcagtcctgacatcgctgtccctggagcatcctccgctggagctggagcttgacaggatcggcttcgccgtcgcccagcgtctggcccaagacggggcccacgtggtagtcagccgccggaagcagcagaatgtggaccaggcagtggccacgctgcagggggaggggctgagcatgacgggcactgtgtgccatgtggggaagatgaaggactgggagcggctggtggccacagtgagctgcagggaaatgggcacagagccaggaggtggaaaagggagccagcctgagcctccttccctgctttcctggacagcattgggcttcagtccttacaatgtcagtaaaacagccttgctgggcctcaacaagaccttggccatagagctggccccaaggaacattagggtgaactgcctagcacctggacttatcaagactagcttcagcaggatgctctggatggacaaggaaaaagaggaaagcatgaaagaaaccctgcggataagaaggttaggcgagccagaggattctcttggcatcgtgtctttcctgtgctctgaagatgccagctacctcactggggaaacagtgatggtgggtggaggaaccccgtcccgcctctgaggacccggagacagcccacaggccagagttgggctctagctcctggtgctgttcctgcattcacccactggcctttcccacctctgctcaccttactgttcacctcatcaaatcagttctgccctgtgaaaagatccagccttccctgccgtcaaggtggtgtcttactcgggattcctgctgttgttgtggccttgggtaaaggcctcccctgagaacacaggacaggcctgctgacaaggctgagtctaccttggcaaagaccaagatattttttgcccaggccactggggaatttgaggggagatgagagagaaggaagctggagtggaaggagcagagttgcaaattaacaacttgcaaatgaggtgcaaataaaatgcagatgattgcgcggctttgaatcgaaaaaaaaaaa

SEQ ID NO: 3 - DNA, 2168 bp, Homo sapiens
  source 1..2168: mol_type = genomic DNA
ggagttagcctcgctcagggcgcggctaaggcgcccagatggcctgcgggcgccaccacgtccctggtcccagctcgggagcacatcagaggcttagaggcgagtgggaagggactcagacagtgcaggacgagaaacgcccgcggcaccaaagcccctcagagcgtcgcccccgcctctagttctagaaagtcagtttcccggcactggcaccccggaacctcaggggctgccgagctgggggggcgctcaagctgcgaggatccgggctgcccgcgagacgaggagcgggcgcccaggatggggtgcatgaagtccaagttcctccaggtcggaggcaatacattctcaaaaactgaaaccagcgccagcccacactgtcctgtgtacgtgccggatcccacatccaccatcaagccggggcctaatagccacaacagcaacacaccaggaatcagggaggcaggctctgaggacatcatcgtggttgccctgtatgattacgaggccattcaccacgaagacctcagcttccagaagggggaccagatggtggtcctagaggaatccggggagtggtggaaggctcgatccctggccacccggaaggagggctacatcccaagcaactatgtcgcccgcgttgactctctggagacagaggagtggtttttcaagggcatcagccggaaggacgcagagcgccaactgctggctcccggcaacatgctgggctccttcatgatccgggatagcgagaccactaaaggaagctactctttgtccgtgcgagactacgaccctcggcagggagataccgtgaaacattacaagatccggaccctggacaacgggggcttctacatatccccccgaagcaccttcagcactctgcaggagctggtggaccactacaagaaggggaacgacgggctctgccagaaactgtcggtgccctgcatgtcttccaagccccagaagccttgggagaaagatgcctgggagatccctcgggaatccctcaagctggagaagaaacttggagctgggcagtttggggaagtctggatggccacctacaacaagcacaccaaggtggcagtgaagacgatgaagccagggagcatgtcggtggaggccttcctggcagaggccaacgtgatgaaaactctgcagcatgacaagctggtcaaacttcatgcggtggtcaccaaggagcccatctacatcatcacggagttcatggccaaaggaagcttgctggactttctgaaaagtgatgagggcagcaagcagccattgccaaaactcattgacttctcagcccagattgcagaaggcatggccttcatcgagcagaggaactacatccaccgagacctccgagctgccaacatcttggtctctgcatccctggtgtgtaagattgctgactttggcctggcccgggtcattgaggacaacgagtacacggctcgggaaggggccaagttccccatcaagtggacagctcctgaagccatcaactttggctccttcaccatcaagtcagacgtctggtcctttggtatcctgctgatggagatcgtcacctacggccggatcccttacccagggatgtcaaaccctgaagtgatccgagctctggagcgtggataccggatgcctcgcccagagaactgcccagaggagctctacaacatcatgatgcgctgctggaaaaaccgtccggaggagcggccgaccttcgaatacatccagagtgtgctggatgacttctacacggccacagagagccagtaccaacagcagccatgatagggaggaccagggcagggccagggggtgcccaggtggtggctgcaaggtggctccagcaccatccgccagggcccacacccccttcctactcccagacacccaccctcgcttcagccacagtttcctcatctgtccagtgggtaggttggactggaaaatctctttttgactcttgcaatccacaatctgacattctcaggaagcccccaagttgatatttctatttcctggaatggttggattttagttacagctgtgatttggaagggaaactttcaaaatagtgaaatgaatatttaaataaaagatataaatgccaaagtctttaccaaaaaaaaaaaaaaaaa

SEQ ID NO: 4 - DNA, 4801 bp, Homo sapiens
  source 1..4801: mol_type = genomic DNA
aaagggggaaaagaaagtgcggcggaaagtaagaggctcactggggaagactgccgggatccaggtctccggggtccgctttggccagaggcgcggaaggaagcagtgcccggcgacactgcacccatcccggctgcttttgctgcgccctctcagcttcccaagaaaggcatcgtcatgtgatcatcacctaagaactagaacatcagcaggccctagaagcctcactcttgcccctccctttaatatctcaaaggatgacacttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtggaactggcttgtggttttggagccacagtcctgctagtggtgattctcattgttgtttaccatgtttactggctagagatggtcctattttaccgggctcattttggaacagatgaaaccattttagatggaaaagagtatgatatttatgtatcctatgcaaggaatgcggaagaagaagaatttgtattactgaccctccgtggagttttggagaatgaatttggatacaagctgtgcatctttgaccgagacagtctgcctgggggaattgtcacagatgagactttgagcttcattcagaaaagcagacgcctcctggttgttctaagccccaactacgtgctccagggaacccaagccctcctggagctcaaggctggcctagaaaatatggcctctcggggcaacatcaacgtcattttagtacagtacaaagctgtgaaggaaacgaaggtgaaagagctgaagagggctaagacggtgctcacggtcattaaatggaaaggggaaaaatccaagtatccacagggcaggttctggaagcagctgcaggtggccatgccagtgaagaaaagtcccaggcggtctagcagtgatgagcagggcctctcgtattcatctttgaaaaatgtatgaaaggaataatgaaaagggtaaaaagaacaaggggtgctccaggaagaaagagtccccccagtcttcattcgcagtttatggtttcataggcaaaaataatggtctaagcctcccaatagggataaatttagggtgactgtgtggctgactattctgcttcctcaggcaacactaaagtttagaaagatatcatcaacgttctgtcaccagtctctgatgccactatgttctttgcaggcaaagacttgttcaatgcgaatttccccttctacattgtctatccctgtttttatatgtctccattctttttaaaatcttaacatatggagcagcctttcctatgaatttaaatatgcctttaaaataagtcactgttgacagggtcatgagtttccgagtatagttttctttttatcttatttttactcgtccgttgaaaagataatcaaggcctacattttagctgaggataatgaacttttttcctcattcggctgtataatacataaccacagcaagactgacatccacttaggatgatacaaagcagtgtaactgaaaatgtttcttttaattgatttaaaggacttgtcttctataccacccttgtcctcatctcaggtaatttatgaaatctatgtaaacttgaaaaatatttcttaatttttgtttttgctccagtcaattcctgattatccacaggtcaacccacattttttcattccttctccctatctgcttatatcgcattgctcatttagagtttgcaggaggctccatactaggttcagtctgaaagaaatctcctaatggtgctatagagagggaggtaacagaaagactcttttagggcatttttctgactcatgaaaagagcacagaaaaggatgtttggcaatttgtcttttaagtcttaaccttgctaatgtgaatactgggaaagtgattttttctcactcgtttttgttgctccattgtaaagggcggaggtcagtcttagtggccttgagagttgcttttggcattaatattctaagagaattaactgtatttcctgtcacctattcactagtgcaggaaatatacttgctccaaataagtcagtatgagaagtcactgtcaatgaaagttgttttgtttgttttcagtaatattttgctgtttttaagacttggaaaactaagtgcagagtttacagagtggtaaatatctatgttacatgtagattatacatatatatacacacgtgtatatgagatatatatcttatatctccacaaacacaaattatatatatacatatccacacacatacattacatatatctgtgtatataaatccacatgcacatgaaatatatatatatatataatttgtgtgtgtgtatgtgtatgtatatgactttaaatagctatgggtacaatattaaaaaccactggaactcttgtccagtttttaaattatgtttttactggaatgtttttgtgtcagtgttttctgtacatattatttgttaattcacagctcacagagtgatagttgtcatagttcttgccttccctaagtttatataaataacttaagtattgctacagtttatctaggttgcagtggcatctgctgtgcacagagcttccatggtcactgctaagcagtagccagccatcgggcattaattgatttcctactatattcccagcagacacatttagaaactaagctatgttaacctcagtgctcaactatttgaactgttgagtgataaaggaaacaaatataactgtaaatgaatcttggtatcctgtgaaacagaataattcgtaatttaagaaagcccttatcccggtaacatgaatgttgatgaacaaatgtaaaattatatcctatatttaagtacccataataaatcatttccctctataagtgttattgattattttaaattgaaaaaagtttcacttggatgaaaaaagtagaaaagtaggtcattcttggatctacttttttttagccttattaatatttttccctattagaaaccacaattactccctctattaacccttcacttactagaccagaaaagaacttattccagataagctttgaatatcaattcttacataaactttaggcaaacagggaatagtctagtcaccaaaggaccattctcttgccaatgctgcattccttttgcacttttggattccatatttatcccaaatgctgttgggcacccctagaaataccttgatgttttttctatttatatgcctgcctttggtacttaattttacaaatgctgtaatataaagcatatcaagtttatgtgatacgtatcattgcaagagaatttgtttcaagatttttttttaatgttccagaagatggccaatagagaacattcaagggaaatggggaaacataatttagagaacaagaacaaaccatgtctcaaatttttttaaaaaaaattaatggttttaaatatatgctatagggacgttccatgcccaggttaacaaagaactgtgatatatagagtgtctaattacaaaatcatatacgatttatttaattctcttctgtattgtaacttagatgattcccaaggactctaataaaaaatcacttcattgtatttggaaacaaaaacatcattcattaattacttattttctttccataggttttaatattttgagagtgtcttttttatttcattcatgaacttttgtatttttcatttttcatttgatttgtaaatttacttatgttaaaaataaaccatttattttcagctttgaattttaaaaaaaaaaaaaaaaaa

SEQ ID NO: 5 - DNA, 1514 bp, Homo sapiens
  source 1..1514: mol_type = genomic DNA
gagccctgcctgcgcccgcccccgaagcggcgcgggacgcctggcgccgtccgcgatccgcagggctgcccgcttaggcttaggcccggcccgctggcaaagccgagccgcagcattttatttcgttcgtggtttccgcacaggctggagtttcgtgggttgggtcgtacttgggacctcggcgaagaggacccgtttatttttttttctttccaaaatggcagcctccagtcgcgcacaagtgttatctctgtaccgggcgatgctgagagagagcaagcgtttcagcgcctacaattacagaacatatgctgtcaggaggataagagatgccttcagagaaaataaaaatgtaaaggatcctgtagaaattcaaaccctagtgaataaagccaagagagaccttggagtaattcgtcgacaggtccacattggccaactgtattcaactgacaagctgatcattgagaatcgagacatgcccaggacctagcaagccggggaccagccaccagtggcggccagggaccaccttcagcatccactctctgtttgagatgggggctcccaaaaccagcttacaatagccttttgcgctgcctgtcctgtgggagctgataaaccaagtcacatttgcattctgttgcaggcttagtgaaaaaggactgctgtctttccttggttcaagtgttagaatggagagctggagttcgttcagaatagtgctgtgtgttaccacgtctcccctgcaccccattcctaccttgtagctcatgaccattgtgtatagcatttctacactttgtttcttggtccttggcaataaaaagaatgatctccctgagcctttgaccccagataaacccctcccaattaatgcattttcatttcctactgatacaaggcctggagagggctgttgggggccctcagggagggttcaactctgagacgagaactgccttggtgaaggcaagttcaagcaccacttgagactgggggcagcatggagtagggcagggctacggggatacacggtgcaccctgcaacttatacctgagcccagtacaacaaaggtgacgggtgtgtaggtacacacccagagatggagcactgcagatcagcaacctcagccccacctgggaatttgctggaaatgcaggctcaagcccctccccacacctggtgaatgagagagccccagcctgacccaagcccagggcgactcccataccctgaagcctggggcatgctgggcagcaccggtgcccaaatctggctggtggacagaagcacctggagagttggagagctttttaaaaagacatctctcagcacttccctctctgcagattctgactcagtaagtgaggggtgaggcacagtcatttttctctattctgaagctctcccactgttttcaatgtttaaccaactggggacccctgctctttaagtatattacaggtaataaagatattgtttgtatgcttttaaaaaaaaaaaaaaaaaa

SEQ ID NO: 6 - DNA, 2379 bp, Homo sapiens
  source 1..2379: mol_type = genomic DNA
gacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaaaaaaaaaaaaaaa

SEQ ID NO: 7 - DNA, 3215 bp, Homo sapiens
  source 1..3215: mol_type = genomic DNA
aacccggggctccgagccggagccgagtctgcgcctgggggaggaccatgcggcagtagcagccatgctgccctttctgctggccacactgggcaccacagccctcaacaacagcaaccccaaggactactgctacagcgcccgcatccgcagcactgtcctgcagggcctgccctttgggggcgtccccaccgtgctggctctcgacttcatgtgcttccttgcactgctgttcttattctctatcctccggaaggtggcctgggactatgggcggctggccttggtgacagatgcagacaggcttcggcggcaggagagggaccgagtggaacaggaatatgtggcttcagctatgcacggggacagccatgaccggtatgagcgtctcacctctgtctccagctccgttgactttgaccaaagggacaatggtttctgttcctggctgacagccatcttcaggataaaggatgatgagatccgggacaaatgtgggggcgatgccgtgcactacctgtcctttcagcggcacatcatcgggctgctggtggttgtgggcgtcctctccgtaggcatcgtgctgcctgtcaacttctcaggggacctgctggagaacaatgcctacagctttgggagaaccaccattgccaacttgaaatcagggaacaacctgctatggctgcacacctccttcgccttcctgtatctgctgctcaccgtctacagcatgcgtagacacacctccaagatgcgctacaaggaggatgatctggtgaagcggaccctcttcatcaatggaatctccaaatatgcagagtcagaaaagatcaagaagcattttgaggaagcctaccccaactgcacagttctcgaagcccgcccgtgttacaacgtggctcgcctaatgttcctcgatgcagagaggaagaaggccgagcggggaaagctgtacttcacaaacctccagagcaaggagaacgtgcctaccatgatcaaccccaagccctgtggccacctctgctgctgtgtggtgcgaggctgtgagcaggtggaggccattgagtactacacaaagctggagcagaagctgaaggaagactacaagcgggagaaggagaaggtgaatgagaagcctcttggcatggcctttgtcaccttccacaatgagactatcaccgccatcatcctgaaggacttcaacgtgtgtaaatgccagggctgcacctgccgtggggagccacgcccctcatcctgcagcgagtccctgcacatctccaactggaccgtgtcctatgcccctgaccctcagaacatctactgggagcacctctccatccgaggcttcatctggtggctgcgctgcctggtcatcaatgtcgtcctcttcatcctcctcttcttcctcaccactccagccatcatcatcaccaccatggacaagttcaacgtcaccaagcctgtggagtacctcaacaaccccatcatcacccagttcttccccaccctgctgctgtggtgcttctcggccctccttcccaccatcgtctactactcagccttctttgaagcccactggacacgctctggggagaacaggacaaccatgcacaagtgctacactttcctcatcttcatggtgctgctcctaccctcgctgggactgagcagcctggacctcttcttccgctggctctttgataagaaattcttggctgaggcagctattcggtttgagtgtgtgttcctgcccgacaacggcgccttcttcgtgaactacgtcattgcctcagcctttatcggcaacgccatggacctgctgcgcatcccaggcctgctcatgtacatgatccggctctgcctggcgcgctcggccgccgagaggcgcaacgtgaagcggcatcaggcctacgagttccagtttggcgcagcctacgcctggatgatgtgcgtcttcacggtggtcatgacctacagtatcacctgccccatcatcgtgcccttcgggctcatgtacatgctgctgaagcacctggtagacaggtacaatctctactacgcctacctgccggccaagctggacaagaagatccactcgggggctgtgaaccaggtggtggccgcgcccatcctctgcctcttctggctgctcttcttttccaccatgcgcacggggttcctagctcccacgtctatgttcacatttgtggtcctggtcatcaccatcgtcatctgtctctgccacgtctgctttggacacttcaaatacctcagtgcccacaactacaagattgagcacacggagacagatactgtggaccccagaagcaatggacggccccccactgctgctgctgtccccaaatctgcgaaatacatcgctcaggtgctgcaggactcagaggtggacggggatggggatggggctcctgggagctcaggggatgagcccccatcatcctcatcccaagatgaggagttgctgatgccacccgacgccctcacggacacagacttccagtcttgcgaggacagcctcatagagaatgagattcaccagtaaggggagggaggggccctggaggccacatcctgccccaccccacccccactcccacggacactaaaacgctaataatttattagatctaaagccccttcctccccagcccctgctttcattaaggtatttaaacttgggggtttcactgctctcccccatgatggagggagggagccccccaacctcagtgaggagagccccgagccggccccggggcaaagaggggtgcagagggagttcccccagatcagtaccccccacccctccccagctagtagcatgaccaggagagggttaatgagagccaagaggagtacctggtgcacctggtgccggtggctggagacctggggggcaggtggatctggggctgttcccccccctccgttttttccaccccacagttcctcctgggatctggccctccagggaagtggagcctccagcccctaggggatgcatgaggggggagggggtgctgagtgggaggaagagtcaggctcacagctggggtggcctgggggtgggggtgggcaaggctgacactggaaaatgggtttttgcactgttttttttttggtttttttgttcttttttgtttttttcctttaaaataaaaacaaagaaaagctctgaaaaaaaaaaaaaaaaa

SEQ ID NO: 8 - DNA, 2581 bp, Homo sapiens
  source 1..2581: mol_type = genomic DNA
ccggggccagggaccagtggtgggaggaggctgcggcgctagatgcggacacctggaccgccgcgccgaggctcccggcgctcgctgctcccgcggcccgcgccatgccctcctacacggtcaccgtggccactggcagccagtggttcgccggcactgacgactacatctacctcagcctcgtgggctcggcgggctgcagcgagaagcacctgctggacaagcccttctacaacgacttcgagcgtggcgcggtggattcatacgacgtgactgtggacgaggaactgggcgagatccagctggtcagaatcgagaagcgcaagtactggctgaatgacgactggtacctgaagtacatcacgctgaagacgccccacggggactacatcgagttcccctgctaccgctggatcaccggcgatgtcgaggttgtcctgagggatggacgcgcaaagttggcccgagatgaccaaattcacattctcaagcaacaccgacgtaaagaactggaaacacggcaaaaacaatatcgatggatggagtggaaccctggcttccccttgagcatcgatgccaaatgccacaaggatttaccccgtgatatccagtttgatagtgaaaaaggagtggactttgttctgaattactccaaagcgatggagaacctgttcatcaaccgcttcatgcacatgttccagtcttcttggaatgacttcgccgactttgagaaaatctttgtcaagatcagcaacactatttctgagcgggtcatgaatcactggcaggaagacctgatgtttggctaccagttcctgaatggctgcaaccctgtgttgatccggcgctgcacagagctgcccgagaagctcccggtgaccacggagatggtagagtgcagcctggagcggcagctcagcttggagcaggaggtccagcaagggaacattttcatcgtggactttgagctgctggatggcatcgatgccaacaaaacagacccctgcacactccagttcctggccgctcccatctgcttgctgtataagaacctggccaacaagattgtccccattgccatccagctcaaccaaatcccgggagatgagaaccctattttcctcccttcggatgcaaaatacgactggcttttggccaaaatctgggtgcgttccagtgacttccacgtccaccagaccatcacccaccttctgcgaacacatctggtgtctgaggtttttggcattgcaatgtaccgccagctgcctgctgtgcaccccattttcaagctgctggtggcacacgtgagattcaccattgcaatcaacaccaaggcccgtgagcagctcatctgcgagtgtggcctctttgacaaggccaacgccacagggggcggtgggcacgtgcagatggtgcagagggccatgaaggacctgacctatgcctccctgtgctttcccgaggccatcaaggcccggggcatggagagcaaagaagacatcccctactacttctaccgggacgacgggctcctggtgtgggaagccatcaggacgttcacggccgaggtggtagacatctactacgagggcgaccaggtggtggaggaggacccggagctgcaggacttcgtgaacgatgtctacgtgtacggcatgcggggccgcaagtcctcaggcttccccaagtcggtcaagagccgggagcagctgtcggagtacctgaccgtggtgatcttcaccgcctccgcccagcacgccgcggtcaacttcggccagtacgactggtgctcctggatccccaatgcgcccccaaccatgcgagccccgccaccgactgccaagggcgtggtgaccattgagcagatcgtggacacgctgcccgaccgcggccgctcctgctggcatctgggtgcagtgtgggcgctgagccagttccaggaaaacgagctgttcctgggcatgtacccagaagagcattttatcgagaagcctgtgaaggaagccatggcccgattccgcaagaacctcgaggccattgtcagcgtgattgctgagcgcaacaagaagaagcagctgccatattactacttgtccccagaccggattccgaacagtgtggccatctgagcacactgccagtctcactgtgggaaggccagctgccccagccagatggactccagcctgcctggcaggctgtctggccaggcctcttggcagtcacatctcttcctccgaggccagtacctttccatttattctttgatcttcagggaactgcatagattgatcaaagtgtaaacaccatagggacccattctacacagagcaggactgcacagcgtcctgtccacacccagctcagcatttccacaccaagcagcaacagcaaatcacgaccactgatagatgtctattcttgttggagacatgggatgattattttctgttctatttgtgcttagtccaattccttgcacatagtaggtacccaattcaattactattgaatgaattaagaattggttgccataaaaataaatcagttcatttaaaatgaaaaaaaaaaaaaaaaaaaa

SEQ ID NO: 9 - DNA, 2272 bp, Homo sapiens
  source 1..2272: mol_type = genomic DNA
agtctccctccagcgggcggcgactccgggttccccctcgcgccctctcgcagaggctcgcccccttccccgcccaccgtccctgcgagcgcgggcggcggcggtgggcgtgtgcgcgcgtgaaggacgccgcctctctctcgctcctgcgttcgcaggcggcggctggcggccggcttctcgctcgggcagcggcggcggcggcggcggcggcttccggagtcccgctgcgaagatgctcaaagtcacggtgccctcctgctccgcctcgtcctgctcttcggtcaccgccagtgcggccccggggaccgcgagcctcgtcccggattactggatcgacggctccaacagggatgcgctgagcgatttcttcgaggtggagtcggagctgggacggggtgctacatccattgtgtacagatgcaaacagaaggggacccagaagccttatgctctcaaagtgttaaagaaaacagtggacaaaaaaatcgtaagaactgagataggagttcttcttcgcctctcacatccaaacattataaaacttaaagagatatttgaaacccctacagaaatcagtctggtcctagaactcgtcacaggaggagaactgtttgataggattgtggaaaagggatattacagtgagcgagatgctgcagatgccgttaaacaaatcctggaggcagttgcttatctacatgaaaatgggattgtccatcgtgatctcaaaccagagaatcttctttatgcaactccagccccagatgcaccactcaaaatcgctgattttggactctctaaaattgtggaacatcaagtgctcatgaagacagtatgtggaaccccagggtactgcgcacctgaaattcttagaggttgtgcctatggacctgaggtggacatgtggtctgtaggaataatcacctacatcttactttgtggatttgaaccattctatgatgaaagaggcgatcagttcatgttcaggagaattctgaattgtgaatattactttatctccccctggtgggatgaagtatctctaaatgccaaggacttggtcagaaaattaattgttttggatccaaagaaacggctgactacatttcaagctctccagcatccgtgggtcacaggtaaagcagccaattttgtacacatggataccgctcaaaagaagctccaagaattcaatgcccggcgtaagcttaaggcagcggtgaaggctgtggtggcctcttcccgcctgggaagtgccagcagcagccatggcagcatccaggagagccacaaggctagccgagacccttctccaatccaagatggcaacgaggacatgaaagctattccagaaggagagaaaattcaaggcgatggggcccaagccgcagttaagggggcacaggctgagctgatgaaggtgcaagccttagagaaagttaaaggtgcagatataaatgctgaagaggcccccaaaatggtgcccaaggcagtggaggatgggataaaggtggctgacctggaactagaggagggcctagcagaggagaagctgaagactgtggaggaggcagcagctcccagagaagggcaaggaagctctgctgtgggttttgaagttccacagcaagatgtgatcctgccagagtactaaacagcttccttcagatctggaagccaaacaccggcattttatgtactttgtccttcagcaagaaaggtgtggaagcatgatatgtactatagtgattctgtttttgaggtgcaaaaaacatacatatataccagttggtaattctaacttcaatgcatgtgactgctttatgaaaataatagtgtcttctatggcatgtaatggatacctaataccgatgagttaaatcttgcaagttaacacaacgtaacacttaaaagcatacattttcagcaaccagtggcacatatttgaagtgaatagtagcaaattgtttttgctttgaaaatctagccatcctacatcctttggatttcttcacaaggcagtaattcctttgaactactgcttagctaatactaggtagtgctaaaagacatgttcccataacttttacaacattttactttttatcattgatgtgttcaaactgtttacaaggagatgcttatagatgatagttgtacatatgtgcaaaaaaaaatccacttgcaatggtaagaaattgaagtatccttaaaggccatgaagccatatgtccctaaaaaaaaaaaaaaaaaaaaaaaaaaaaaaaaa

SEQ ID NO: 10 - DNA, 2175 bp, Homo sapiens
  source 1..2175: mol_type = genomic DNA
gttgtatatcagggccgcgctgagctgcgccagctgaggtgtgagcagctgccgaagtcagttccttgtggagccggagctgggcgcggattcgccgaggcaccgaggcactcagaggaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaaaaaaaaaaaaaaaaaa

SEQ ID NO: 11 - DNA, 2403 bp, Homo sapiens
  source 1..2403: mol_type = genomic DNA
ggggccttgccttccgcactcgggcgcagccgggtggatctcgagcaggtgcggagccccgggcggcgggcgcgggtgcgagggatccctgacgcctctgtccctgtttctttgtcgctcccagcctgtctgtcgtcgttttggcgcccccgcctccccgcggtgcggggttgcacaccgatcctgggcttcgctcgatttgccgccgaggcgcctcccagacctagaggggcgctggcctggagcagcgggtcgtctgtgtcctctctcctctgcgccgcgcccggggatccgaagggtgcggggctctgaggaggtgacgcgcggggcctcccgcaccctggccttgcccgcattctccctctctcccaggtgtgagcagcctatcagtcaccatgtccgcagcctggatcccggctctcggcctcggtgtgtgtctgctgctgctgccggggcccgcgggcagcgagggagccgctcccattgctatcacatgttttaccagaggcttggacatcaggaaagagaaagcagatgtcctctgcccagggggctgccctcttgaggaattctctgtgtatgggaacatagtatatgcttctgtatcgagcatatgtggggctgctgtccacaggggagtaatcagcaactcagggggacctgtacgagtctatagcctacctggtcgagaaaactattcctcagtagatgccaatggcatccagtctcaaatgctttctagatggtctgcttctttcacagtaactaaaggcaaaagtagtacacaggaggccacaggacaagcagtgtccacagcacatccaccaacaggtaaacgactaaagaaaacacccgagaagaaaactggcaataaagattgtaaagcagacattgcatttctgattgatggaagctttaatattgggcagcgccgatttaatttacagaagaattttgttggaaaagtggctctaatgttgggaattggaacagaaggaccacatgtgggccttgttcaagccagtgaacatcccaaaatagaattttacttgaaaaactttacatcagccaaagatgttttgtttgccataaaggaagtaggtttcagagggggtaattccaatacaggaaaagccttgaagcatactgctcagaaattcttcacggtagatgctggagtaagaaaagggatccccaaagtggtggtggtatttattgatggttggccttctgatgacatcgaggaagcaggcattgtggccagagagtttggtgtcaatgtatttatagtttctgtggccaagcctatccctgaagaactggggatggttcaggatgtcacatttgttgacaaggctgtctgtcggaataatggcttcttctcttaccacatgcccaactggtttggcaccacaaaatacgtaaagcctctggtacagaagctgtgcactcatgaacaaatgatgtgcagcaagacctgttataactcagtgaacattgcctttctaattgatggctccagcagtgttggagatagcaatttccgcctcatgcttgaatttgtttccaacatagccaagacttttgaaatctcggacattggtgccaagatagctgctgtacagtttacttatgatcagcgcacggagttcagtttcactgactatagcaccaaagagaatgtcctagctgtcatcagaaacatccgctatatgagtggtggaacagctactggtgatgccatttccttcactgttagaaatgtgtttggccctataagggagagccccaacaagaacttcctagtaattgtcacagatgggcagtcctatgatgatgtccaaggccctgcagctgctgcacatgatgcaggaatcactatcttctctgttggtgtggcttgggcacctctggatgacctgaaagatatggcttctaaaccgaaggagtctcacgctttcttcacaagagagttcacaggattagaaccaattgtttctgatgtcatcagaggcatttgtagagatttcttagaatcccagcaataatggtaacattttgacaactgaaagaaaaagtacaaggggatccagtgtgtaaattgtattctcataatactgaaatgctttagcatactagaatcagatacaaaactattaagtatgtcaacagccatttaggcaaataagcactcctttaaagccgctgccttctggttacaatttacagtgtactttgttaaaaacactgctgaggcttcataatcatggctcttagaaactcaggaaagaggagataatgtggattaaaaccttaagagttctaaccatgcctactaaatgtacagatatgcaaattccatagctcaataaaagaatctgatacttagaccaaaaaaaaaaa

SEQ ID NO: 12 - DNA, 1315 bp, Homo sapiens
  source 1..1315: mol_type = genomic DNA
ctactctgtcaccgcccctgggaagagtggaacccatacttgctggtctgatccatgcacaaggcggggctgctaggcctctgtgcccgggcttggaattcggtgcggatggccagctccgggatgacccgccgggacccgctcgcaaataaggtggccctggtaacggcctccaccgacgggatcggcttcgccatcgcccggcgtttggcccaggacggggcccatgtggtcgtcagcagccggaagcagcagaatgtggaccaggcggtggccacgctgcagggggaggggctgagcgtgacgggcaccgtgtgccatgtggggaaggcggaggaccgggagcggctggtggccacggctgtgaagcttcatggaggtatcgatatcctagtctccaatgctgctgtcaaccctttctttggaagcataatggatgtcactgaggaggtgtgggacaagactctggacattaatgtgaaggccccagccctgatgacaaaggcagtggtgccagaaatggagaaacgaggaggcggctcagtggtgatcgtgtcttccatagcagccttcagtccatctcctggcttcagtccttacaatgtcagtaaaacagccttgctgggcctgaccaagaccctggccatagagctggccccaaggaacattagggtgaactgcctagcacctggacttatcaagactagcttcagcaggatgctctggatggacaaggaaaaagaggaaagcatgaaagaaaccctgcggataagaaggttaggcgagccagaggattgtgctggcatcgtgtctttcctgtgctctgaagatgccagctacatcactggggaaacagtggtggtgggtggaggaaccccgtcccgcctctgaggaccgggagacagcccacaggccagagttgggctctagctcctggtgctgttcccgcattcacccactggcctttcccacctctgctcaccttactgttcacctcatcaaatcagttctgccctgtgaaaagatccagccttccctgccgtcaaggtggcgtcttactcgggatttctgctgttgttgtggccttgggtaaaggcctcccctgagaacacaggacaggcctgctgacaaggctgagtctaccttggcaaagaccaagatattttttcccgggccactggggaatctgaggggtgatgggagagaaggaacctggagtggaaggagcagagttgcaaattaacaacttgcaaatgaggtgcaaataaaatgcagatgattgcgcggctttgaatccaaaaaaaaaaaaaaaaaa

SEQ ID NO: 13 - DNA, 3661 bp, Homo sapiens
  source 1..3661: mol_type = genomic DNA
cccaagactgtccccgctggaggcggtagagggatccagaagtaatgagatgctaatgagtcgcgaataaagcccgggcggcgccccgcgcccctcgcggaagcccacactccgcgcgactccaggcgcacgccccgggccgccccgcatcccagcatccccgcccgatctcggcgtttccgcccccgcccccgcccccgccctcccacccgctcagacctggttgccagcccaacaggaagcggcccctcccggcttcggagccgccgccactcatctctgcccagctgctgccctccccaggaggcctccatggcttcacctacctccaccaacccagcgcatgcccactttgagagcttcctgcaggcccagctgtgccaggacgtgctgagcagcttccaggagctgtgtggggccctggggctggaacccggtggggggctgccccagtaccacaagatcaaggaccagctcaactactggagcgccaagtcactgtggaccaagctggacaagcgagcaggccagcctgtctaccagcagggccgggcctgcaccagcaccaagtgcctggtggtgggtgctggaccttgcgggctgcgggtcgctgtggagctggcgctgctgggggcccgagtggtgctggtggaaaagcgcaccaagttctctcgccacaacgtgctccacctctggcccttcaccatccacgacctgcgggcactcggtgctaagaagttctacgggcgcttctgcaccggcaccctggaccacatcagcatcaggcagctccagctgcttctgctgaaggtagcattgctgctgggggtggaaattcactggggtgtcactttcactggcctccagccccctcctaggaaggggagtggctggcgtgcccagctccaacccaacccccctgcccagctggccaactatgaatttgacgtccttatctcggctgcaggaggtaaattcgtccctgaaggcttcaaagttcgagaaatgcgaggcaaactggccattggcatcacagccaactttgtgaatggacgcaccgtggaggagacacaggtgccggagatcagtggtgtagccaggatctacaaccagagcttcttccagagccttctcaaagccacaggcattgatctggagaacattgtgtactacaaggacgacacccactactttgtgatgacagccaagaagcagtgcctgctgcggctgggggtgctgcgccaggactggccagacaccaatcggctgctgggcagtgccaatgtggtgcccgaggctctgcagcgctttacccgggcagctgctgactttgccacccatggcaagctcgggaaactagagtttgcccaggatgcccatgggcagcctgatgtctctgcctttgacttcacgagcatgatgcgggcagagagttctgctcgtgtgcaagagaagcatggcgcccgcctgctgctgggactggtgggggactgcctggtggagcccttctggcccctgggcactggagtggcacggggcttcctggcagcctttgatgcagcctggatggtgaagcggtgggcagagggcgctgagtccctagaggtgttggctgagcgtgagagcctgtaccagcttctgtcacagacatccccagaaaacatgcatcgcaatgtggcccagtatgggctggacccagccacccgctaccccaacctgaacctccgggcagtgacccccaatcaggtacgagacctgtatgatgtgctagccaaggagcctgtgcagaggaacaacgacaagacagatacagggatgccagccaccgggtcggcaggcacccaggaggagctgctacgctggtgccaggagcagacagctgggtacccgggagtccacgtctccgatttgtcttcctcctgggctgatgggctagctctgtgtgccctggtgtaccggctgcagcctggcctgctggaaccctcagagctgcaggggctgggagctctggaagcaactgcttgggcactaaaggtggcagagaatgagctgggcatcacaccggtggtgtctgcacaggccgtggtagcagggagtgacccactgggcctcattgcctacctcagccacttccacagtgccttcaagagcatggcccacagcccaggccctgtcagccaggcctccccagggacctccagtgctgtattattccttagtaaacttcagaggaccctgcagcgatcccgggccaaggaaaatgcagaggatgctggtggcaagaagctgcgcttggagatggaggccgagaccccaagtactgaggtgccacctgacccagagcctggtgtacccctgacacccccatcccaacaccaggaggccggtgctggggacctgtgtgcactttgtggggaacacctctatgtcctggaacgcctctgtgtcaacggccatttcttccaccggagctgcttccgctgccatacctgtgaggccacactgtggccaggtggctacgagcagcacccaggagatggacatttctactgcctccagcacctgccccagacagaccacaaagcggaaggcagcgatagaggccctgagagtccggagctccccacaccaagtgagaatagcatgccaccaggcctctcaactcccacagcctcgcaggagggggccggtcctgttccagatcccagccagcccacccgtcggcagatccgcctctccagcccggagcgccagcggttgtcctcccttaaccttacccctgacccggaaatggagcctccacccaagcctccccgcagctgctccgccttggcccgccacgccctggagagcagctttgtgggctggggcctgccagtccagagccctcaagctcttgtggccatggagaaggaggaaaaagagagtcccttctccagtgaagaggaagaagaagatgtgcctttggactcagatgtggaacaggccctgcagacctttgccaagacctcaggcaccatgaataactacccaacatggcgtcggactctgctgcgccgtgcgaaggaggaggagatgaagaggttctgcaaggcccagaccatccaacggcgactaaatgagattgaggctgccttgagggagctagaggccgagggcgtgaagctggagctggccttgaggcgccagagcagttccccagaacagcaaaagaaactatgggtaggacagctgctacagctcgttgacaagaaaaacagcctggtggctgaggaggccgagctcatgatcacggtgcaggaattgaatctggaggagaaacagtggcagctggaccaggagctacgaggctacatgaaccgggaagaaaacctaaagacagctgctgatcggcaggctgaggaccaggtcctgaggaagctggtggatttggtcaaccagagagatgccctcatccgcttccaggaggagcgcaggctcagcgagctggccttggggacaggggcccagggctagacgagggtgggccgtctgctttcgttcccacaaagaaagcacctcaccccagcacagtgccacccctgttcatctgggctgcctggcagagagccttgctgtttacaattaaaatgtttctgccacaaaaaaaaaaaaaaaaaaa

SEQ ID NO: 14 - DNA, 651 bp, Homo sapiens
  source 1..651: mol_type = genomic DNA
atgtccatagccctgaagcaggtattcaacaaggacaagaccttccgacccaagaggaaatttgaacctggcacacagaggtttgagctgcacaaacgggctcaggcatccctcaactcgggtgtggacctgaaggcggctgtgcagttgcccagtggggaggaccagaatgactgggtggcagtacatgtggtggacttcttcaatcggatcaacctcatctatggcaccatctgtgagttctgcaccgagcggacctgtcctgtgatgtcagggggccccaaatatgagtatcggtggcaggatgatctcaagtataagaagccaacagcgctgccagctccccagtacatgaaccttcttatggattggattgaggttcagatcaacaacgaggaaatatttccaacatgcgtgggtgttcccttcccaaagaacttccttcagatctgcaagaagatcctgtgccgccttttccgggtctttgtccacgtctatatccaccacttcgaccgggtcattgtgatgggtgcagaggcccatgtcaacacctgctacaaacacttctattactttgtcacagagatgaacctcatagaccgcaaggagctagagcctttgaaagaaatgacgagcaggatgtgtcactaa

SEQ ID NO: 15 - DNA, 2471 bp, Homo sapiens
  source 1..2471: mol_type = genomic DNA
gcgttacaggccctttggcgcctgcgtattcgtgaagtgtgaaaaaagcgcgcctctgttgggacgggaaatcagcctttctattggtcagggttagaaaccccgcctttgaggcattttcaaccaatggaagcgcggcattcttcatttaaactgtctataaatttctgcctagtcaaagttaagagtggcgccagggatttgaaccgcgctgacgaagtttggtgatccatcttccgagtatcgccgggatttcgaatcgcgatgatcatcccctctctagaggagctggactccctcaagtacagtgacctgcagaacttagccaagagtctgggtctccgggccaacctgagggcaaccaagttgttaaaagccttgaaaggctacattaaacatgaggcaagaaaaggaaatgagaatcaggatgaaagtcaaacttctgcatcctcttgtgatgagactgagatacagatcagcaaccaggaagaagctgagagacagccacttggccatgtcaccaaaacaaggagaaggtgcaagactgtccgtgtggaccctgactcacagcagaatcattcagagataaaaataagtaatcccactgaattccagaatcatgaaaagcaggaaagccaggatctcagagctactgcaaaagttccttctccaccagacgagcaccaagaagctgagaatgctgtttcctcaggtaacagagattcaaaggtaccttcagaaggaaagaaatctctctacacagatgagtcatccaaacctggaaaaaataaaagaactgcaatcactactccaaactttaagaagcttcatgaagctcattttaaggaaatggagtccattgatcaatatattgagagaaaaaagaaacattttgaagaacacaattccatgaatgaactgaagcagcagcccatcaataagggaggggtcaggactccagtacctccaagaggaagactctctgtggcttctactcccatcagccaacgacgctcgcaaggccggtcttgtggccctgcaagtcagagtaccttgggtctgaaggggtcactcaagcgctctgctatctctgcagctaaaacgggtgtcaggttttcagctgctactaaagataatgagcataagcgttcactgaccaagactccagccagaaagtctgcacatgtgaccgtgtctgggggcaccccaaaaggcgaggctgtgcttgggacacacaaattaaagaccatcacggggaattctgctgctgttattaccccattcaagttgacaactgaggcaacgcagactccagtctccaataagaaaccagtgtttgatcttaaagcaagtttgtctcgtcccctcaactatgaaccacacaaaggaaagctaaaaccatgggggcaatctaaagaaaataattatctaaatcaacatgtcaacagaattaacttctacaagaaaacttacaaacaaccccatctccagacaaaggaagagcaacggaagaaacgcgagcaagaacgaaaggagaagaaagcaaaggttttgggaatgcgaaggggcctcattttggctgaagattaataattttttaacatcttgtaaatattcctgtattctcaacttttttccttttgtaaattttttttttttgctgtcatccccactttagtcacgagatctttttctgctaactgttcatagtctgtgtagtgtccatgggttcttcatgtgctatgatctctgaaaagacgttatcaccttaaagctcaaattctttgggatggtttttacttaagtccattaacaattcaggtttctaacgagacccatcctaaaattctgtttctagatttttaatgtcaagttcccaagttccccctgctggttctaatattaacagaactgcagtcttctgctagccaatagcatttacctgatggcagctagttatgcaagcttcaggagaatttgaacaataacaagaatagggtaagctgggatagaaaggccacctcttcactctctatagaatatagtaacctttatgaaacggggccatatagtttggttatgacatcaatattttacctaggtgaaattgtttaggcttatgtaccttcgttcaaatatcctcatgtaattgccatctgtcactcactatattcacaaaaataaaactctacaactcattctaacattgcttacttaaaagctacatagccctatcgaaatgcgaggattaatgctttaatgcttttagagacagggtctcactgtgttgcccaggctggtctcaaactccaccaaatgtacttcttattcattttatggaaaagactaggctttgcttagtatcatgtccatgtttccttcacctcagtggagcttctgagttttatactgctcaagatcgtcataaataaaattttttctcattgtcatagaaaaaaaaaaaaaaaaaa

SEQ ID NO: 16 - DNA, 2685 bp, Homo sapiens
  source 1..2685: mol_type = genomic DNA
gcggaggcggcctgccggggtggttcggcttcccgttgccgcctcgggcgctgtacccagagctcgaagaggagcagcgcggccgcgcggacccggcaaggctgggccggactcggggctcccgagggacgccatgcggggaggcaggggcgcccctttctggctgtggccgctgcccaagctggcgctgctgcctctgttgtgggtgcttttccagcggacgcgtccccagggcagcgccgggccactgcagtgctacggagttggacccttgggcgacttgaactgctcgtgggagcctcttggggacctgggagccccctccgagttacacctccagagccaaaagtaccgttccaacaaaacccagactgtggcagtggcagccggacggagctgggtggccattcctcgggaacagctcaccatgtctgacaaactccttgtctggggcactaaggcaggccagcctctctggccccccgtcttcgtgaacctagaaacccaaatgaagccaaacgccccccggctgggccctgacgtggacttttccgaggatgaccccctggaggccactgtccattgggccccacctacatggccatctcataaagttctgatctgccagttccactaccgaagatgtcaggaggcggcctggaccctgctggaaccggagctgaagaccatacccctgacccctgttgagatccaagatttggagctagccactggctacaaagtgtatggccgctgccggatggagaaagaagaggatttgtggggcgagtggagccccattttgtccttccagacaccgccttctgctccaaaagatgtgtgggtatcagggaacctctgtgggacgcctggaggagaggaacctttgcttctatggaaggccccagggccctgtgtgcaggtgagctacaaagtctggttctgggttggaggtcgtgagctgagtccagaaggaattacctgctgctgctccctaattcccagtggggcggagtgggccagggtgtccgctgtcaacgccacaagctgggagcctctcaccaacctctctttggtctgcttggattcagcctctgccccccgtagcgtggcagtcagcagcatcgctgggagcacggagctactggtgacctggcaaccggggcctggggaaccactggagcatgtagtggactgggctcgagatggggaccccctggagaaactcaactgggtccggcttccccctgggaacctcagtgctctgttaccagggaatttcactgtcggggtcccctatcgaatcactgtgaccgcagtctctgcttcaggcttggcctctgcatcctccgtctgggggttcagggaggaattagcacccctagtggggccaacgctttggcgactccaagatgcccctccagggacccccgccatagcgtggggagaggtcccaaggcaccagcttcgaggccacctcacccactacaccttgtgtgcacagagtggaaccagcccctccgtctgcatgaatgtgagtggcaacacacagagtgtcaccctgcctgaccttccttggggtccctgtgagctgtgggtgacagcatctaccatcgctggacagggccctcctggtcccatcctccggcttcatctaccagataacaccctgaggtggaaagttctgccgggcatcctattcttgtggggcttgttcctgttggggtgtggcctgagcctggccacctctggaaggtgctaccacctaaggcacaaagtgctgccccgctgggtctgggagaaagttcctgatcctgccaacagcagttcaggccagccccacatggagcaagtacctgaggcccagccccttggggacttgcccatcctggaagtggaggagatggagcccccgccggttatggagtcctcccagcccgcccaggccaccgccccgcttgactctgggtatgagaagcacttcctgcccacacctgaggagctgggccttctggggccccccaggccacaggttctggcctgaaccacacgtctggctgggggctgccagccaggctagagggatgctcatgcaggttgcaccccagtcctggattagccctcttgatggatgaagacactgaggactcagagaggctgagtcacttacctgaggacacccagccaggcagagctgggattgaaggacccctatagagaagggcttggcccccatggggaagacacggatggaaggtggagcaaaggaaaatacatgaaattgagagtggcagctgcctgccaaaatctgttccgctgtaacagaactgaatttggaccccagcacagtggctcacgcctgtaatcccagcactttggcaggccaaggtggaaggatcacttagagctaggagtttgagaccagcctgggcaatatagcaagacccctcactacaaaaataaaacatcaaaaacaaaaacaattagctgggcatgatggcacacacctgtagtccgagccacttgggaggctgaggtgggaggatcggttgagcccaggagttcgaagctgcagggacctctgattgcaccactgcactccaggctgggtaacagaatgagaccttatctcaaaaataaacaaactaataaaaagcaaaaaaaaaaaaaaaaaaaaaaaaaaaa

SEQ ID NO: 17 - DNA, 622 bp, Homo sapiens
  source 1..622: mol_type = genomic DNA
cataaaccctggcgcgctcgcgggccggcactcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtagccgttcctcctgcccgctgggcctcccaacgggccctcctcccctccttgcaccggcccttcctggtctttgaataaagtctgagtgggcagcaaaaaaaaaaaaaaaaaa

SEQ ID NO: 18 - DNA, 5199 bp, Homo sapiens
  source 1..5199: mol_type = genomic DNA
agggacgggaagtgggcggggccggccggcagcagcttgcgggacacggagccgcgaggagacagctgaggcccgcggagaccagggggtgaagcctggagaccctcttgccctggcctagctgcaggcccccgggatgctttgggcatgtcctctggagccccacagaagagcagcccaatggccagtggagctgaggagaccccaggcttcctggacacgctcctgcaagacttcccagccctgctgaacccagaggaccctctgccatggaaggccccagggacggtgctcagccaggaggaggtggagggcgagctggctgagctggccatgggctttctgggcagcaggaaggccccgccaccacttgctgctgctctggcccacgaagcagtttcacagctgctacagacagacctttccgaattcaggaagttgcccagggaggaagaagaagaggaggaggacgatgacgaggaggaaaaggcccctgtgaccttgctggatgcccaaagcctggcacagagtttctttaaccgcctttgggaagtcgccggccagtggcagaagcaggtgccattggctgcccgggcctcacagcggcagtggctggtctccatccacgccatccggaacactcgccgcaagatggaggaccggcacgtgtccctcccttccttcaaccagctcttcggcttgtctgaccctgtgaaccgcgcctactttgctgtgtttgatggtcacggaggcgtggatgctgcgaggtacgccgctgtccacgtgcacaccaacgctgcccgccagccagagctgcccacagaccctgagggagccctcagagaagccttccggcgcaccgaccagatgtttctcaggaaagccaagcgagagcggctgcagagcggcaccacaggtgtgtgtgcgctcattgcaggagcgaccctgcacgtcgcctggctcggggattcccaggtcattttggtacagcagggacaggtggtgaagctgatggagccacacagaccagaacggcaggatgagaaggcgcgcattgaagcattgggtggctttgtgtctcacatggactgctggagagtcaacgggaccctggccgtctccagagccatcggggatgtcttccagaagccctacgtgtctggggaggccgatgcagcttcccgggcgctgacgggctccgaggactacctgctgcttgcctgtgatggcttctttgacgtcgtaccccaccaggaagttgttggcctggtccagagccacctgaccaggcagcagggcagcgggctccgtgtcgccgaggagctggtggctgcggcccgggagcggggctcccacgacaacatcacggtcatggtggtcttcctcagggacccccaagagctgctggagggcgggaaccagggagaaggggacccccaggcagaagggaggaggcaggacttgccctccagccttccagaacctgagacccaggctccaccaagaagctaggtggtttccaggcccctgccctccccttcctcccatccttgtccttctctccctcagaagcctcaggacccaacaggtggcaggcagtggacagggtgcccgccccacagtgctttccccagcaccccagagccagtcgggacaccccccgcagcccgtcctggtggctgtggaactgcactgggtggcgggcagatggtggaaggcagcttaggagacctcaccaaagagaagatggaccggctcttgctcccagctcctattaggcccggggtgggaccagaggtcataggtgcccaacggcagccaaaccaaagacactggtgtgcatggggcagcatggttgtgcacgtgggaccctggggcggacccaggagccaaactcttgaagcaccccctgggtcaggcccagcagcggagtggccagccccagtttcccattgctcctctctgcggccagggccaggtgggttcatatttacagatatgcccagccagtcctggtcggccacaccagtgtcccaaagaggagagcgcagcagagccaggggtctgttctgtagcagccacccccctgcccccactccagggcagccatgatgtgcttgggcccaccagggccttccgggctgctctcttccctgagcccggaaccggcgacgcacatgtgtcttttgttggtgtgtttgtttttttccagggaggtctaattccgaagcagtattccaggttttctctttgttttatcagtgccaagatgacctgttgtgtcatataatttaagcagagcttagcatttattttattctttagaaaacttaagtatttacttttttaaagctatttttcaaggaacctttttttgcagtattattgaatttattttctaaatcaggattgaaacaggaacttttccaggtggtgttaataagccattcaagtgccttacacagctttgaagaaactaggactgcagtgggctcggataggcccattgaggtttttagaaaagcaggatttgttttgttagggaggcatgattttggtgagatctttctggaagagttttccgcctctttgtgatgctgaacacccccaaggttctcccctccccccgctgcccaggtgactggcaggagctgcgactgccacgtagtggtgcctgggcccgacagcggggctctgggcatcccgggtgaccttggcccatctgcctgcattcccacccccttgggcctggctggatcccaggcagagggaccttgctgctgtgtgattggaacattcccaaatatcttgtgaatttgtaatcaaattggtctcattgggaaagactcttaattaagaggctcaggcaagcacagaggcagcccgtgggtctctgtctcagtctggaggcagcagggatgctgctgggagtccatggcacaggccacagcccctcaccttgccgcggtggctggcagcacgcctgccttgctctgccccatgccctgaacaggcatgagagctccacgtcccctagtgcaccctgagagggggctcacaagtgaccgatcctgggtgcctcagggagctcactgagggcgtgcaaagttgaaagtggcaaggctgggggagggtgtcgggtagagggaagagggcagggggctaggggaggactcagaggccatctgcagggccaagccacaggaagggctgagctggaggtgggcagggctgctccaggcaggtcagagcagtgcagggggaggagaggagaaagggaggaagctgggctgtgtggtccccatgaaggcattcagagtccacctgcagacagcgagagccccaggaaggtttgcacagctgtgccccaagcaccttggcctcctctcagctcgccgaggaggcacgctagagccgccttcccggtgggagccctctgtcccacagggagcggggagccagctttgctggggccctacctgcatgcccagccttacccctcattctcacagcacagatgaggttgagaccatgcagtcaatgcattgcttaaggtctcttatttacaaaaaaaaaccttaaacatagtcgctgtcattcagacattcagagaatggttggccacaaacaatgaccaagtattgcttggcttaacttgaaggcctgctgtctccttctgggggtcagggacgcagctccaccctcaccactagcccaccctgcccgtgggcataaccttgacgaagagagagaatgattggcatctgcttttctcttttctttgctaataattctgttcctggctgccgagagtgaagtttcaccatgtggaggtttggctcctatcacctggtggtctgattcataccctagcctgaggctccactggaagatctcgcagcctcagtgtatgggaaaccctttccccaggcttgtcccagcactgccgctccccacccctgagccaggaccccagaggatggccatgccccgtgcctggcagaggtctggtgccagcactgggagctgctccgcccttgccttggggccgagggagccctcgtccacccctgcacagcagctgggcacagaggagcgctcttccatcttgaccaggactgcaccaagaagcaccaggtgtcttcagcctccaacctccggggcgaccttctcttccagccacagtcccatgagggcccctagccagggacactggtctgtaaattgtaatcctttctccagcccagctctccacttgttccttgtgtgagctgagcaggcagtgcacctctgagtgtcccttttgtaaggcccaggggttgcactgagtctgcagaggccgcgacctcctagaacgctgtgggtgcaggtgagccggcgtgtcctggggagatgctgccagcacacaggggccctcctgctgccagcaggttggggtggttaagtcttattagtgtctattcttaaaattaagtgggctggagaagaatggagctccacatgccagcaccgtatatggaatacaaaagctggggaagcagggcctgccttacaggtgtggctgactctgagcccaggcctgcaggggtggagggcagtccctcagaatcccagaggcagtcccagcctcagaacccaggataggaaatgggtgtgtttagtggggaaagggacggggtgcagacggcagggccagtatggggccccctccctctcctctcctctcctatggtgagcccagcgtgggcaccgggccgtctcagccgtgttcccagggctgggaggacagctctggcccttcttaggcctagcctcgtcccaagctaaatgtaagccagttgggctgtgttaaaggaagcagtgtttttggttcgattctgcctctgtagctcaaggggggcagcccccagagtcctgtgcattctgccaaggctccatagctttgccaaatgcacggagctctgccattccggtgcagtgcaggccttgcgaagggtttatctgcgttcgtctcggtgggcttctcctgcatgggagttgtgttcctgtgcaagggggagctttgctccaggacaggatgactgtcttccctattcttagggacaagtcccaagatgccagaaaggcagtctcccaaggacccaccatgcagaagtgtcaataaaccacaagttctgaactctgtaaaaaaaaaaaaaa

SEQ ID NO: 19 - DNA, 1767 bp, Homo sapiens
  source 1..1767: mol_type = genomic DNA
atggcggcggccgacggcgacgactcgctgtaccccatcgcggtgctcatagacgaactccgcaatgaggacgttcagcttcgcctcaacagcatcaagaagctgtccaccatcgccttggcccttggggttgaaaggacccgaagtgagcttctgcctttccttacagataccatctatgatgaagatgaggtcctcctggccctggcagaacagctgggaaccttcactaccctggtgggaggcccagagtacgtgcactgcctgctgccaccgctggagtcgctggccacagtggaggagacagtggtgcgggacaaggcagtggagtccttacgggccatctcacacgagcactcgccctctgacctggaggcgcactttgtgccgctagtgaagcggctggcgggcggcgactggttcacctcccgcacctcggcctgcggcctcttctccgtctgctacccccgagtgtccagtgctgtgaaggcggaacttcgacagtacttccggaacctgtgctcagatgacacccccatggtgcggcgggccgcagcctccaagctgggggagtttgccaaggtgctggagctggacaacgtcaagagtgagatcatccccatgttctccaacctggcctctgacgagcaggactcggtgcggctgctggcggtggaggcgtgcgtgaacatcgcccagcttctgccccaggaggatctggaggccctggtgatgcccactctgcgccaggccgctgaagacaagtcctggcgcgtccgctacatggtggctgacaagttcacagagctccagaaagcagtggggcctgagatcaccaagacagacctggtccctgccttccagaacctgatgaaagactgtgaggccgaggtgagggccgcagcctcccacaaggtcaaagagttctgtgaaaacctctcagctgactgtcgggagaatgtgatcatgtcccagatcttgccctgcatcaaggagctggtgtccgatgccaaccaacatgtcaagtctgccctggcctcagtcatcatgggtctctctcccatcttgggcaaagacaacaccatcgagcacctcttgcccctcttcctggctcagctgaaggatgagtgccctgaggtacggctgaacatcatctctaacctggactgtgtgaacgaggtgattggcatccggcagctgtcccagtccctgctccctgccattgtggagctggctgaggacgccaagtggcgggtgcggctggccatcattgagtacatgcccctcctggctggacagctgggagtggagttctttgatgagaaacttaactccttgtgcatggcctggcttgtggatcatgtatatgccatccgcgaggcagccaccagcaacctgaagaagctagtggaaaagtttgggaaggagtgggcccatgccacaatcatccccaaggtcttggccatgtccggagaccccaactacctgcaccgcatgactacgctcttctgcatcaatgtgctgtctgaggtctgtgggcaggacatcaccaccaagcacatgctacccacggttctgcgcatggctggggacccggttgccaatgtccgcttcaatgtggccaagtctctgcagaagatagggcccatcccggacaacagcaccttgcagagtgaagtcaagcccatcctagagaagctgacccaggaccaggatgtggacgtcaaatactttgcccaggaggctctgactgttctgtctctcgcc

SEQ ID NO: 20 - DNA, 10640 bp, Homo sapiens
  source 1..10640: mol_type = genomic DNA
atactcagtcacacaagccatagcaggaaacagcgagcttgcagcctcaccgacgagtctcaactaaaagggactcccggagctaggggtggggactcggcctcacacagtgagtgccggctattggacttttgtccagtgacagctgagacaacaaggaccacgggaggaggtgtaggagagaagcgccgcgaacagcgatcgcccagcaccaagtccgcttccaggctttcggtttctttgcctccatcttgggtgcgccttcccggcgtctaggggagcgaaggctgaggtggcagcggcaggagagtccggccgcgacaggacgaactcccccactggaaaggattctgaaagaaatgaagtcagccctcagaaatgaagttgactgcctgctggctttctgttgactggcccggagctgtactgcaagacccttgtgagcttccctagtctaagagtaggatgtctgctgaagtcatccatcaggttgaagaagcacttgatacagatgagaaggagatgctgctctttttgtgccgggatgttgctatagatgtggttccacctaatgtcagggaccttctggatattttacgggaaagaggtaagctgtctgtcggggacttggctgaactgctctacagagtgaggcgatttgacctgctcaaacgtatcttgaagatggacagaaaagctgtggagacccacctgctcaggaaccctcaccttgtttcggactatagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggctccataatgggagaagtaaagaacaaagacttaaggaacagcttggcgctcaacaagaaccagtgaagaaatccattcaggaatcagaagcttttttgcctcagagcatacctgaagagagatacaagatgaagagcaagcccctaggaatctgcctgataatcgattgcattggcaatgagacagagcttcttcgagacaccttcacttccctgggctatgaagtccagaaattcttgcatctcagtatgcatggtatatcccagattcttggccaatttgcctgtatgcccgagcaccgagactacgacagctttgtgtgtgtcctggtgagccgaggaggctcccagagtgtgtatggtgtggatcagactcactcagggctccccctgcatcacatcaggaggatgttcatgggagattcatgcccttatctagcagggaagccaaagatgttttttattcagaactatgtggtgtcagagggccagctggaggacagcagcctcttggaggtggatgggccagcgatgaagaatgtggaattcaaggctcagaagcgagggctgtgcacagttcaccgagaagctgacttcttctggagcctgtgtactgcggacatgtccctgctggagcagtctcacagctcaccatccctgtacctgcagtgcctctcccagaaactgagacaagaaagaaaacgcccactcctggatcttcacattgaactcaatggctacatgtatgattggaacagcagagtttctgccaaggagaaatattatgtctggctgcagcacactctgagaaagaaacttatcctctcctacacataagaaaccaaaaggctgggcgtagtggctcacacctgtaatcccagcactttgggaggccaaggagggcagatcacttcaggtcaggagttcgagaccagcctggccaacatggtaaacgctgtccctagtaaaaatacaaaaattagctgggtgtgggtgtgggtacctgtattcccagttacttgggaggctgaggtgggaggatcttttgaacccaggagttcagggtcatagcatgctgtgattgtgcctacgaatagccactgcataccaacctgggcaatatagcaagatcccatctctttaaaaaaaaaaaaaaaggacaggaactatcttactcaatgtattagtcatgtttctctagagggacagaactaataggatacatgtatataaaaaggggagtttattaaggagtattgactcacatgatcacagggttaggtcccacaataggtcatctgcaagcaaggaagccaattcaagtcccaaagctgaagaacttggagtccaatgtttgagggcaggaagcattcagcatgagagaaagatggaggccagaagactacaccagtctagtctttccatgttttgcctgcttttattctggcagtgctggcagctgattagatggtgcccacccagattgaggatggtctgcctttcccagtccactgactcaaatgttaaatctcctttggcagcaccctcacagatgtacccgggaacactttgcatccttctattcaatcaagttgatactcagtattaaccatcacagtccatttgggcaactataccaaattaccatagaccaggtgacttaaacagcagttatttctcacagttccggaggctgggaaatccaacatctaagtggtagcatatctggtgtctggtaaggcatgcttccagatcttaccagatgtcagtcttttgatgttctcacatggcagaaaaagaggatgcaaactctcaagtatatctttaagggcacaaattccattcatgagggctctaccctcatcacctaattacctcccaaaggccccaccttctgatactgtcactttggggatactgtctcccctttgaattctggggggaatacaaacattcagtttgtaacaatagccttatgatttagaggttacttgttcattcacctagacctcaaattgcattttacagctagtcaagtatatctttctctgatttgatagtgtgacctaaaaggggaccattgtttgaaatatcattagagttgcttattattattattattattattattattattattattattattattgagacagagtttcattctgctgcccaggctggagtgcagtggcatcatcttggctcattgcaacctctgccttctgggttcaagcgattctcctgcctcagcctcccgagtagctgggattacaggctcctgccaccacacccggctaatttttgtatttttagtggagacagggtttccaccatgttggccagcgtggtcttgaactcctgacctcaggtgattcaccagcctcggcctcccaaagtgctgggattacaggtgtgagccactgcacctggcctattattatttttaaattttttttttttaattgatcattcttgggtgtttctcacagagggtgatttggcagggtcacaggacaatagtggagggaaggtcagcagataaacaagtgaacaaaggtctctggttttcctaggcagaggaccctgcggccttccgcagtgtttgtgtccctgggtacttgagattagggagtggtgatgactcttaaggagcatgctgccttcaagcatctgtttaacaaagcacatcttgcactgcccttaatccatttaaccctgagtggacacagcacatgtttcagagagcacagggttgggggtaaggtcatagatcaacagcatcctaaggcagaagaatttttcttagtacagaacaaaatgaagtctcccatgtctacttctttctacacagacacagcaacaatctgatttctctatcttttccccacctttcccccttttctattccacaaaaccgccatcgtcatcatggcctgttctcaatgagctgttgggtacacctcccagacggggtggcggctgggcagaggggctcctcacttcccagatggggcggccaggcggacgcgccccccacctccctcccggacgggatagctggccgggcgggggctgaccccccacctccctccccgacggggcggctggccgggcgggggctgacccccacgcctccctcccggacggggcggctgccaggcggaggggctcctcacttctcagacggggtggctgctgggcggagacgctcctcacttcccagacagggtggctgtcgggcggaggggctcctcacttctcagacggggcagctgcgggcggaggggctcctcacttctcagacggggtggccgggcagagaagctcctcacatcccagacgggggggcggggcagaggcgctccccacatctcagacgatgggcggccgggcagagacgctcctcacttcatcccagacggggtggcggccgggcagaagctgtaatctcggcaccctggggggccaaggcaggcggctgggaggcggaggccgtagccagctgagatcacaccactgcactccagcctgggcaacattgagcactgagtggacgagactctgcccgcaatcccggcacctcgggaggccgaggctggcagatcactcgcagtcaggagctggagaccagcccggccaacacagtgaaaccctgtctccaccaaaaaaatacgaaaaccagtcaggcgtggcggcgcccgcaatggcaggcacgcggcaggccgaggcgggagaatcaggcagggaggctgcagtgagccgagatggcagcagtacagtccagcttcggctcggcatcagagggagaccgtggggagagggagaagagagggagggggagagggctatttttaaaattttttaaaattgctgaacaggggtacctctgggcagtgtgtcagaataccactttttaaatattttatgatttatttatttttctatttcttgaggttttaactgatgtgtatctgtatgtctatttgtgtatattttgtcatgatcatgtaacagagtctgaaaagtgtcgaagagacagttttcaggaacaacaagcaattattcctactttccaagttattttgatgccatggtggctcatacctataatctgagtactttgggaggctgaggtggactgatcacttgagcccaggagtttgagaccagcctgggcaacatagcaagactccatctctacaaaaaaagacaaaatttagctgagcgtggtggcgtgttcctgtagtcccagctacttgggaggctgaagtgagtggatcccctgagcccagagaggtcaaggttgtgatgagctgtgatcacaccactgcacttcagcatgggagacagagtgagaccctgtttcagaaaaaataaataaataaaaccaccagcaccacaaacaacaacaaaaagttattttgtacttgttttgagcacaggactcctgagggtatctttgcatttaatattacataggggtgccagtgggaagtaatgtgtatgcttggcctcatgagctaaaaccctgtgttaattatgacagaaggaaagtgtgtgagagagatcttaactacctagcagctctagctgccatcttgaaccatgaagatacgggccacacgtaggggtagctgggtagtgagcagcaagaagccttgttggatgagggcacgaaggagcagaatcactggaatcactgtgtcagccctaattacctacctctggacttttatgtgaggggaaaaaaaattgacagtttatatttatctcaacctagttaacccaagtgatgcattgttatgagattaaaatgtttggaggccgggtgcggtggctcacgcctataatcccagccctttgggaggccaaggcgggcggatcacgaggtcaggagatcaagaccatcctggctaacatgtaaaaccccgtctctactaaaaatacaaaaaattagccaggcgttgtggcggtcgcctgtagtccctgctatttgggaggccgaggcaagagaacggcatgaacctgggaggtggagcttgcagcgagctgagatcttgccactgcactccagcctgggcgacagtgcgagactctgtctcaaaaataaataaataaataaataataaataaaatgtttggaatgttggcttcatccctgggatgcaaggctggttcaacatacgcaaatcaagaaacataattcatcacataaacagaactaaagacaaaaaccacatgattatctcaatagatacagaaaaggccttcaataaaattcaacgttgcttcatgttaaaaactctcaataaactaggtattgatggaaaatatctcaaaataataaccatttatgacaaacccacagccattatcatactgaatgggcaaaagctggaagcattccccttgaaaactggcacaagacagggatgccgtctcaccactcctatttaacatagtattggaagttctggccaagaaaatcaggcaagagaaacaaataaggggtattcaaataggaaaagaggaagtaaaactgtgtttgcagatgacatgatactatatctagaaaaccccattatctccacccaaaagttccttaagctgataagcaacttcagcaaagtctcaggatacaaaatcaatgtgcagaaatcacaagcattctatacaccaacaatacacaagcagagagccaaatcatgaatgaactcccattcacagttgctagaaagagaataaaatacctaggaatacagctaataagatgtgaaggatctcttcaaggagaactacaaaccactgctcaaggaaataagagaggacacaaatgaaaaaacattccattctcgtggataggaagaatcaatatcatgaaaatggccatactacccaaagtaatttataggttcattgctattcccattaaactactattgacattcttcacagaattagaaaaaaactactttaaaattcaaatggaaccaaaaaagagcccgtataaccaagacaacaataagcaaaaagaacaaagctggaagcatcacactacccaacttcaaagtatactgcaaggctacagtagccaaaatggcatggtactggtacaaaaacagacacatagaccaatggaacagaatagagaccagagaaagaagaccacacatctacagccatctgatcatcgacaaacctgacaaaaacaagcaatggggaaaagattccctatttaataaatggtgctgggaaaactggctagccatatgcagaaaattgaaactgaccccttccttacaccttatacaaaaattaactcaagattaaagacttaatgtaaaacctaaaactataaaaaccctagaagaaaatctatttaataccattcaagacataggcacaagcaaaggtttcatgacaaaaacatcaaaagcaattgcaacaaaagcaaaaattacaaatgggatctaattaaactaaagagctcctgcacagcaaaagaaactatcattagagtgaacaggcaacctacagaatgggagaacatttttgcaatctatccatctgacaaaggtctaatatccagaacctacaaggaacttaaaacaaatttacaaggaaaaaaacaaccccatcaaaaagtggacaaaggacatgaacagacacttctcaaaagaagacatttatgtggccaacaaacatataaaaaaaagctcaaccttactgatcattagagaaatgcaaaggagaaccacaatgagataccatctcatgccggtcagaatggtgattattaaaaagtcaaaaaacaacagatgctggcgaggctgtggagaagtaggaacacttttacattgttggtgggaatgtaaattagttcaaccgttgtggaagtgtgtgtggctattcctcaaagatctagaactagaaatactatttgtcccagcaatcccattactgggtatatacccaaaggaatataaaccattttattataaagatacatgcacatttttgttcattgcagcactcttcacaatagcaaagacacaatagcaaatgcccatcaaagatagactggataaagaaaatgtggtacatatacaccatggaatactgtgcagtgcagccattacagcttttggtgatacagtgaatcagatttttcattaattcttttaattggttattactgaacgtgaaaaagtaatgtttgtattgaaatcttgagtctggccatgtttctattttaaattcataaagaattctaacaagaggaattccaagaatgtcataaatggatgtttctccatggatgaaggaactgttttattcacttgctgataattcagcctaatccagtttgacatcatatagataagtagttgaattatggatttaaaatacatatcattttctaactccaaaggtaatacttatttaaatggttttgaaaatatagaaaggcacaatttctttttaaatctgttattctccaccaccactcaatctgtctatcatctatctctccattcattcttccatttgtttatatctgttaatctttgtatgtgttcatgtatagcttttacatgattggaatcataatgcatattccattttgaagtctgcttttttttacacaaaaatatgttgtgaatattttcctatattatgaaatatcattagctgagcttttagaattgactgcatgttttggtaccatttagatatagtttaagatacttagaagttatgtggctttgccactatggatgaatcttatttactcaatattaattacttacaaataacctcacctaaacactactcagccataaaaaggaatgaattaatgacattcacagcaacctggagactattactctaaaggaagtaactgaggaatggaaaaccaaacattgtatgttctcactcataagtgggagataagctatgaggatgcaaaggcataagaaggatacaatggactttggggacttaggggaaagggtgggaggggggtgaaggataaaagaatacaaattgggttcagtgtatactgctcaggtgatgggtgcaccagaatctcacaagtaaccacttaattacttacgcatgtaaccagataccacctgttccccaaacacctatggaaataattttgtttttttttttaaaaaaggaatgagatcatgtcctttgcagggacatggatgaagctggaagccattatcctcagcaaactaacagaggagcaggaaaccaaacaccacatgttctcacttgtaagcggaagctgaacaatgagaacacacggacacagggatgagatcaacacacactggggcctgatgcaggggccgtagcggggagagcatcaggataactagctaatgcatgtggggcttaatacctaggtgataggttgataggtgcagcaaaccaccatgggacacgtttacctatgtaacaaacccgcacatcctgcacttgtatccagaacttaaaatattttaaaaatctttagagaatacaaaaaaaaaaaaaaagattcttcaatgcatacacaataaaattgcagttcagtcaaacattggaagtctttctctgactgtctagttggtatcttcattttcagcttcttcaagatcccactccaaacactgttagctcagccaaattgaacagctcatatctcctacctctggatctttggttctggtgattgtatatttctggaccatctggaaccccagcatatcaccctaccccacatctccacatccccaaaatataaccatacttcaagggcagttcaaataccatctccttctatcctccatgaagtcagttatctcttccattggaattatcgccccctctcctgaacagtactatttcgtgtgaatctcctccaagccttcttttcattttatatctcatgctgtaattcttggaaagtatgctgtagctcaagtgcagaattctcatcagttttatctttatatctctcctaaacactttacctgatgaagagcctggcatacacataaatatatattgaatgaatcagtgatggattgaaaagagaaatgatggatctcctaaattttaacttttataaaatattttgatacattcatgaccttactttagcaagcaatgaacgtgatgtaaactattgttgatatagtttttatattggaagtgtaagtagtttgtggcatgggattgtgacatatcctaggtttcctcatcttctttttattgaaatgtaattcacaagccataaaatttgcccctttaaagtaaatgatgcagtggattttagtatatttacagagttgtgcaatcatcaccactatctaattccagaacatttccatctacctagaaactccataccagtgagctgccactctaatcctcctcttcccccagcctctagaaacaataatccattttctgtctctatgatttgcctgttctagatattttataaaaataaacatgtggcctttcgtgtctgacttccttcacttaaaaaaaaaaaaaaaaaa

SEQ ID NO: 21 - DNA, 1939 bp, Homo sapiens
  source 1..1939: mol_type = genomic DNA
cgcctccgccttcggaggctgacgcgcccgggcgccgttccaggcctgtgcagggcggatcggcagccgcctggcggcgatccagggcggtgcggggcctgggcgggagccgggaggcgcggccggcatggaggcgctgctgctgggcgcggggttgctgctgggcgcttacgtgcttgtctactacaacctggtgaaggccccgccgtgcggcggcatgggcaacctgcggggccgcacggccgtggtcacgggcgccaacagcggcatcggaaagatgacggcgctggagctggcgcgccggggagcgcgcgtggtgctggcctgccgcagccaggagcgcggggaggcggctgccttcgacctccgccaggagagtgggaacaatgaggtcatcttcatggccttggacttggccagtctggcctcggtgcgggcctttgccactgcctttctgagctctgagccacggttggacatcctcatccacaatgccggtatcagttcctgtggccggacccgtgaggcgtttaacctgctgcttcgggtgaaccatatcggtccctttctgctgacacatctgctgctgccttgcctgaaggcatgtgcccctagccgcgtggtggtggtagcctcagctgcccactgtcggggacgtcttgacttcaaacgcctggaccgcccagtggtgggctggcggcaggagctgcgggcatatgctgacactaagctggctaatgtactgtttgcccgggagctcgccaaccagcttgaggccactggcgtcacctgctatgcagcccacccagggcctgtgaactcggagctgttcctgcgccatgttcctggatggctgcgcccacttttgcgcccattggcttggctggtgctccgggcaccaagagggggtgcccagacacccctgtattgtgctctacaagagggcatcgagcccctcagtgggagatattttgccaactgccatgtggaagaggtgcctccagctgcccgagacgaccgggcagcccatcggctatgggaggccagcaagaggctggcagggcttgggcctggggaggatgctgaacccgatgaagacccccagtctgaggactcagaggccccatcttctctaagcaccccccaccctgaggagcccacagtttctcaaccttaccccagccctcagagctcaccagatttgtctaagatgacgcaccgaattcaggctaaagttgagcctgagatccagctctcctaaccctcaggccaggatgcttgccatggcacttcatggtccttgaaaacctcggatgtgtgcgaggccatgccctggacactgacgggtttgtgatcttgacctccgtggttactttctggggccccaagctgtgccctggacatctcttttcctggttgaaggaataatgggtgattatttcttcctgagagtgacagtaaccccagatggagagataggggtatgctagacactgtgcttctcggaaatttggatgtagtattttcaggccccacccttattgattctgatcagctctggagcagaggcagggagtttgcaatgtgatgcactgccaacattgagaattagtgaactgatccctttgcaaccgtctagctaggtagttaaattacccccatgttaatgaagcggaattaggctcccgagctaagggactcgcctagggtctcacagtgagtaggaggagggcctgggatctgaacccaagggtctgaggccagggccgactgccgtaagatgggtgctgagaagtgagtcagggcagggcagctggtatcgaggtgccccatgggagtaaggggacgccttccgggcggatgcagggctggggtcatctgtatctgaagcccctcggaataaagcgcgttgaccgccgaaaaaaaaaaaaaaaaaaaa

SEQ ID NO: 22 - DNA, 1840 bp, Homo sapiens
  source 1..1840: mol_type = genomic DNA
gggttcccaggccgactctccttgtggttggctgaggctggaggtggacgggacttttggagggtcgctcgcgtctgttcgcagagctgtgggcggagttgaggccttggaggctgagatgtggttctgcgcgtgtgcggacggctgtctgttaactccgcggtcagttcccggactggtggctggtctgcagggttgacctgcgcaatgcagaggctgcaggtagtgctgggccacctgaggggtccggccgattccggctggatgccgcaggccgcgccttgcctgagcggtgccccgcaggcctcggccgcggacgtggtggtggtgcacgggcggcgcacggccatctgccgggcgggccgcggcggcttcaaggacaccacccccgacgagcttctctcggcagtcatgaccgcggttctcaaggacgtgaatctgaggccggaacagctgggggacatctgtgtcggaaatgtgctgcagcctggggccggggcaatcatggcccgaatcgcccagtttctgagtgacatcccggagactgtgcctttgtccactgtcaatagacagtgttcgtcggggctacaggcagtggccagcatagcaggtggcatcagaaatgggtcttatgacattggcatggcctgtggggtggagtccatgtccctggctgacagagggaaccctggaaatattacttcgcgcttgatggagaaggagaaggccagagattgcctgattcctatggggataacctctgagaatgtggctgagcggtttggcatttcacgggagaagcaggatacctttgccctggcttcccagcagaaggcagcaagagcccagagcaagggctgtttccaagctgagattgtgcctgtgaccaccacggtccatgatgacaagggcaccaagaggagcatcactgtgacccaggatgagggtatccgccccagcaccaccatggagggcctggccaaactgaagcctgccttcaagaaagatggttctaccacagctggaaactctagccaggtgagtgatggggcagctgccatcctgctggcccggaggtccaaggcagaagagttgggccttcccatccttggggtcctgaggtcttatgcagtggttggggtcccacctgacatcatgggcattggacctgcctatgccatcccagtagctttgcaaaaagcagggctgacagtgagtgacgtggacatcttcgagatcaatgaggcctttgcaagccaggctgcctactgtgtggagaagctacgactcccccctgagaaggtgaaccccctggggggtgcagtggccttagggcacccactgggctgcactggggcacgacaggtcatcacgctgctcaatgagctgaagcgccgtgggaagagggcatacggagtggtgtccatgtgcatcgggactggaatgggagccgctgccgtctttgaataccctgggaactgagtgaggtcccaggctggaggcgctacgcagacagtcctgctgctctagcagcaaggcagtaacaccacaaaagcaaaaccacatgggaaaactcagcactggtggtggtggcagtggacagatcaaggcacttcaactcatttggaaaatgtgaacactgatgacatggtataggagtgggtggggtgttgagccacccatcagaccctctttagctgtgcaagataaaagcagcctgggtcacccaggccacaaggccatggttaattcttaaggcaaggcaaatccatggatgagaagtgcaatgggcatagtaaaagtgcatgaatttatcttaaaaaaaaaaaaaaaaaaaaaa

SEQ ID NO: 23 - DNA, 3407 bp, Homo sapiens
  source 1..3407: mol_type = genomic DNA
caggttgaaatggctgatgacatcactggttcccgggagcggtagagctggagccggagccaagggagtccaggctgccgggggctgcagacatggagggccagagcagcaggggcagcaggaggccagggacccgggctggcctgggttccctgcccatgccccagggtgttgcccaaactggggcaccctccaaggtggactcaagttttcagctcccagcaaagaagaacgcagccctaggaccctcggaaccaaggttggctctggcacctgtagggccacgggcagctatgtcagcttcctcggaaggaccgaggctggctctggcatctccccgaccaatcctggctccactgtgtacccctgaagggcagaaaacagctactgcccaccgcagctccagcctggccccaacatctgtgggccagctggtgatgtctgcctcagctggaccaaagcctcccccagcgaccacaggctcagttctggctccgacgtccctggggctggtgatgcctgcctcagcagggccaagatctcccccagtcaccctggggcccaatctggccccaacctccagagaccagaagcaggagccacctgcctccgtgggacccaagccaacactggcagcctctggcctgagcctggccctggcttctgaggagcagcccccagaactcccctccaccccttccccggtgcccagtccagttctgtctccaactcaggaacaggccctggctccagcatccacggcatcaggcgcagcctctgtgggacagacatcagctagaaagagggatgccccagcccctagacctctccctgcttctgaggggcatctccagcctccagctcagacatctggtcctacaggctccccaccctgcatccaaacctccccagaccctcggctctccccctccttccgagcccggcctgaggccctccacagcagccctgaggatcctgttttgccacggccaccccagaccttgcccttggatgtgggccagggtccttcagagcctggcactcactcccctggacttctgtcccccaccttccggcctggggccccctcaggccagactgtgcccccacctctgcccaagccaccccgatcacccagccgttccccaagccactccccgaatcgctctccctgtgttcccccagcccctgacatggccctcccaaggcttggcacacagagtacagggcctggcaggtgcctgagccccaaccttcaggcccaagaagccccagccccagtcaccacctcctcttctacatccaccctgtcatcctccccttggtcagctcagcctacctggaagagcgaccccggcttccggatcactgtggtcacatggaacgtgggcactgccatgcccccagacgatgtcacatccctcctccacctgggcggtggtgacgacagcgacggcgcagacatgatcgccatagggttgcaggaagtgaactccatgctcaacaagcgactcaaggacgccctcttcacggaccagtggagtgagctgttcatggatgcgctagggcccttcaacttcgtgctggtgagttcggtgaggatgcagggtgtcatcctgctgctgttcgccaagtactaccacctgcccttcctgcgagacgtgcagaccgactgcacgcgcactggcctgggcggctactggggtaacaagggtggcgtgagcgtgcgcctggcggccttcgggcacatgctctgcttcctgaactgccacttgcctgcgcatatggacaaggcggagcagcgcaaagacaacttccagaccatcctcagcctccagcagttccaagggccgggcgcacagggcatcctggatcatgacctcgtgttctggttcggggacctgaacttccgcattgagagctatgacctgcactttgtcaagtttgccatcgacagtgaccagctccatcagctctgggagaaggaccagctcaacatggccaagaacacctggcccattctgaagggctttcaggaggggcccctcaacttcgctcccaccttcaagtttgatgtgggtaccaacaaatacgataccagtgccaagaaacggaagccagcttggacagaccgtatcctatggaaggtcaaggctccaggtgggggtcccagcccctcaggacggaagagccaccgactccaggtgacgcagcacagctaccgcagccacatggaatacacagtcagcgaccacaagcctgtggctgcccagttcctcctgcagtttgccttcagggacgacatgccactggtgcggctggaggtggcagatgagtgggtgcggcccgagcaggcggtggtgaggtaccgcatggaaacagtgttcgcccgcagctcctgggactggatcggcttataccgggtgggtttccgccattgcaaggactatgtggcttatgtctgggccaaacatgaagatgtggatgggaatacctaccaggtaacattcagtgaggaatcactgcccaagggccatggagacttcatcctgggctactatagtcacaaccacagcatcctcatcggcatcactgaacccttccagatctcgctgccttcctcggagttggccagcagcagcacagacagctcaggcaccagctcagagggagaggatgacagcacactggagctccttgcacccaagtcccgcagccccagtcctggcaagtccaagcgacaccgcagccgcagcccgggactggccaggttccctgggcttgccctacggccctcatcccgtgaacgccgtggtgccagccgtagcccctcaccccagagccgccgcctgtcccgagtggctcctgacaggagcagtaatggcagcagccggggcagtagtgaagaggggccctctgggttgcctggcccctgggccttcccaccagctgtgcctcgaagcctgggcctgttgcccgccttgcgcctagagactgtagaccctggtggtggtggctcctggggacctgatcgggaggccctggcgcccaacagcctgtctcctagtccccagggccatcgggggctggaggaagggggcctggggccctgagggtggggtaggcagatgggccaaggtgaccaccattctgcctcaatcttttgcaagcccacctgcctctctcctgctgctcctccagctgtatctgcacctgcctctctgtcctggccaggggtggacaactggggtcccccaaaactcagtcctggcacctcaactgtgacaatcagcaaagccccacccaggcccccatctgggatgatgggagagctctggcagatgtcccaatcctggaggtcatccattaggaattaaattctccagcctcaaaaaaaaaaaaaaaaa

SEQ ID NO: 24 - DNA, 1146 bp, Homo sapiens
  source 1..1146: mol_type = genomic DNA
ttttgcgaacggcgagcagcggcggcggcgcggagagacgcagcggaggttttcctggtttcggaccccagcggccggatggtgaaatcctccctgcagcggatcctcaatagccactgcttcgccagagagaaggaaggggataaacccagcgccaccatccacgccagccgcaccatgccgctcctaagcctgcacagccgcggcggcagcagcagtgagagttccagggtctccctccactgctgtagtaacccgggtccggggcctcggtggtgctcctgatgcccctcacccacccctgaagatcccaggtgggcgagggaatagtcagagggatcacaatctttcagctaacttattctactccgatgatcggctgaatgtaacagaggaactaacgtccaacgacaagacgaggattctcaacgtccagtccaggctcacagacgccaaacgcattaactggcgaacagtgctgagtggcggcagcctctacatcgagatcccgggcggcgcgctgcccgaggggagcaaggacagctttgcagttctcctggagttcgctgaggagcagctgcgagccgaccatgtcttcatttgcttccacaagaaccgcgaggacagagccgccttgctccgaaccttcagctttttgggctttgagattgtgagaccggggcatccccttgtccccaagagacccgacgcttgcttcatggcctacacgttcgagagagagtcttcgggagaggaggaggagtagggccgcctcggggctgggcatccggcccctggggccaccccttgtcagccgggtgggtaggaaccgtagactcgctcatctcgcctgggtttgtccgcatgttgtaatcgtgcaaataaacgctcactccgaattagcggtgtatttcttgaagtttaatattgtgtttgtgatactgaagtatttgctttaattctaaataaaaatttatattttacttttttattgctggtttaagatgattcagattatccttgtactttgaggagaagtttcttatttggagtcttttggaaacagtcttagtcttttaacttggaaagatgaggtattaatcccctccattgctctccaaaagccaataaagtgattacacccga

SEQ ID NO: 25 - DNA, 1215 bp, Homo sapiens
  source 1..1215: mol_type = genomic DNA
gccaggaaggcttgcaggttctgctgtttggttgctgaagggggtcagtgtgtgtatgtgtcatggaggtgggcagggaaggggagggctgtgcgtgggggagaggatatatatgctggtgtggctgagaaagcggaaccgagcctcgcatccatcggagggagccggggactgacagctctcagcacctgcttcctgctcctgcaccatgaaagtcctgctttgtgacctgctgctgctcagtctcttctccagtgtgttcagcagttgtcagagggactgtctcacatgccaggagaagctccacccagccctggacagcttcgacctggaggtgtgcatcctcgagtgtgaagagaaggtcttccccagccccctctggactccatgcaccaaggtcatggccaggagctcttggcagctcagccctgccgccccagagcatgtggcggctgctctctaccagccgagagcttcggagatgcagcatctgcggcgaatgccccgagtccggagcttgttccaggagcaggaagagcccgagcctggcatggaggaggctggtgagatggagcagaagcagctgcagaagagatttgggggcttcaccggggcccggaagtcggccaggaagttggccaatcagaagcggttcagtgagtttatgaggcaatacttggtcctgagcatgcagtccagccagcgccggcgcaccctgcaccagaatggtaatgtgtagccggaaggggcgctcctcccagctgtaccggccactgcaacccatgagcgtccaggtgatcccccaaacagcatgtgctcagccccagacctgccgcctgggaatcaggattccttcttccccaaggcactgagcgcctgcagatcccgcaggcttcgtttgcctccagaaccttcccgtctgattgttcctccccagccccctggcatgtttcaccacaaccctgttgctacatcagagtgtatttttgtaattcctctagctaccatttcaatagccccatctctcctgctcacccgcctcttgccccttctaggggcaggtgaaaggaataggaaattgaacctggggttttgacttgccactgccataacttgtttgtaaaagagctgttctttttgactgattgttttaaacaacgatttctccattaaacttctactgagcaaatggttaataaaaaaaaaaaaaaaaaa

SEQ ID NO: 26 - DNA, 4264 bp, Homo sapiens
  source 1..4264: mol_type = genomic DNA
agagcgctgcggccgcggcggtgcagcagaggcgcctcgggcaggaggagggcggcttctgcgagggcagcctgaggtattaaaaagtgtcagcaaactgcattgaataacagacatcctaagaggggatattttccacctctataatgaagaaaagcaggagtgtgatgacggtgatggctgatgataatgttaaagattattttgaatgtagcttgagtaaatcctacagttcttccagtaacacacttgggatcgacctctggagagggagaaggtgttgctcaggaaacttacagttaccaccactgtctcaaagacagagtgaaagggcaaggactcctgagggagatggtatttccaggccgaccacactgcctttgacaacgcttccaagcattgctattacaactgtaagccaggagtgctttgatgtggaaaatggcccttccccaggtcggagtccactggatccccaggccagctcttccgctgggctggtacttcacgccacctttcctgggcacagccagcgcagagagtcatttctctacagatcagacagcgactatgacttgtcaccaaaggcgatgtcgagaaactcttctcttccaagcgagcaacacggcgatgacttgattgtaactccttttgcccaggtccttgccagcttgcgaagtgtgagaaacaacttcactatactgacaaaccttcatggtacatctaacaagaggtccccagctgctagtcagcctcctgtctccagagtcaacccacaagaagaatcttatcaaaaattagcaatggaaacgctggaggaattagactggtgtttagaccagctagagaccatacagacctaccggtctgtcagtgagatggcttctaacaagttcaaaagaatgctgaaccgggagctgacacacctctcagagatgagccgatcagggaaccaggtgtctgaatacatttcaaatactttcttagacaagcagaatgatgtggagatcccatctcctacccagaaagacagggagaaaaagaaaaagcagcagctcatgacccagataagtggagtgaagaaattaatgcatagttcaagcctaaacaatacaagcatctcacgctttggagtcaacactgaaaatgaagatcacctggccaaggagctggaagacctgaacaaatggggtcttaacatctttaatgtggctggatattctcacaatagacccctaacatgcatcatgtatgctatattccaggaaagagacctcctaaagacattcagaatctcatctgacacatttataacctacatgatgactttagaagaccattaccattctgacgtggcatatcacaacagcctgcacgctgctgatgtagcccagtcgacccatgttctcctttctacaccagcattagacgctgtcttcacagatttggagatcctggctgccatttttgcagctgccatccatgacgttgatcatcctggagtctccaatcagtttctcatcaacacaaattcagaacttgctttgatgtataatgatgaatctgtgttggaaaatcatcaccttgctgtgggtttcaaactgctgcaagaagaacactgtgacatcttcatgaatctcaccaagaagcagcgtcagacactcaggaagatggttattgacatggtgttagcaactgatatgtctaaacatatgagcctgctggcagacctgaagacaatggtagaaacgaagaaagttacaagttcaggcgttcttctcctagacaactataccgatcgcattcaggtccttcgcaacatggtacactgtgcagacctgagcaaccccaccaagtccttggaattgtatcggcaatggacagaccgcatcatggaggaatttttccagcagggagacaaagagcgggagaggggaatggaaattagcccaatgtgtgataaacacacagcttctgtggaaaaatcccaggttggtttcatcgactacattgtccatccattgtgggagacatgggcagatttggtacagcctgatgctcaggacattctcgataccttagaagataacaggaactggtatcagagcatgatacctcaaagtccctcaccaccactggacgagcagaacagggactgccagggtctgatggagaagtttcagtttgaactgactctcgatgaggaagattctgaaggacctgagaaggagggagagggacacagctatttcagcagcacaaagacgctttgtgtgattgatccagaaaacagagattccctgggagagactgacatagacattgcaacagaagacaagtcccccgtggatacataatccccctctccctgtggagatgaacattctatccttgatgagcatgccagctatgtggtagggccagcccaccatgggggccaagacctgcacaggacaagggccacctggcctttcagttacttgagtttggagtcagaaagcaagaccaggaagcaaatagcagctcaggaaatcccacggttgacttgccttgatggcaagcttggtggagagggctgaagctgttgctgggggccgattctgatcaagacacatggcttgaaaatggaagacacaaaactgagagatcattctgcactaagtttcgggaacttatccccgacagtgactgaactcactgactaataacttcatttatgaatcttctcacttgtccctttgtctgccaacctgtgtgccttttttgtaaaacattttcatgtctttaaaatgcctgttgaatacctggagtttagtatcaacttctacacagataagctttcaaagttgacaaacttttttgactctttctggaaaagggaaagaaaatagtcttccttctttcttgggcaatatccttcactttactacagttacttttgcaaacagacagaaaggatacacttctaaccacattttacttccttcccctgttgtccagtccaactccacagtcactcttaaaacttctctctgtttgcctgcctccaacagtacttttaactttttgctgtaaacagaataaaattgaacaaattagggggtagaaaggagcagtggtgtcgttcaccgtgagagtctgcatagaactcagcagtgtgccctgctgtgtcttggaccctgccccccacaggagttgtacagtccctggccctgttccctacctcctctcttcaccccgttaggctgttttcaatgtaatgctgccgtccttctcttgcactgccttctgcgctaacacctccattcctgtttataaccgtgtatttattacttaatgtatataatgtaatgttttgtaagttattaatttatatatctaacattgcctgccaatggtggtgttaaatttgtgtagaaaactctgcctaagagttacgactttttcttgtaatgttttgtattgtgtattatataacccaaacgtcacttagtagagacatatggcccccttggcagagaggacaggggtgggcttttgttcaaagggtctgccctttccctgcctgagttgctacttctgcacaacccctttatgaaccagttttggaaacaatattctcacattagatactaaatggtttatactgagcttttacttttgtatagcttgataggggcagggggcaatgggatgtagtttttacccaggttctatccaaatctatgtgggcatgagttgggttataactggatcctactatcattgtggctttggttcaaaaggaaacactacatttgctcacagatgattcttctgaatgctcccgaactactgactttgaagaggtagcctcctgcctgccattaagcaggaatgtcatgttccagttcattacaaaagaaaacaataaaacaatgtgaatttttataataaaatgtgaactgatgtagcaaattacgcaaatgtgaagcctcttctgataacacttgttaggcctcttactgatgtcagtttcagtttgtaaaatatgtttcatgctttcagttcagcattgtgactcagtaattacagaaaatggcacaaatgtgcatgaccaatgtatgtctatgaacactgcattgtttcaggtggacattttatcattttcaaatgtttctcacaatgtatgttatagtattattattatatattgtgttcaaatgcattctaaagagacttttatatgaggtgaataaagaaaagcatgattagattaaaaaaa

SEQ ID NO: 27 - DNA, 2759 bp, Homo sapiens
  source 1..2759: mol_type = genomic DNA
gctcaggccccgcccctgccgccggaatcctgaagcccaaggctgcccgggggcggtccggcggcgccggcgatggggcataaaaccactggccacctgccgggctgctcctgcgtgcgctgccgtcccggatccaccgtgcctctgcggcctgcgtgcccggagtccccgcctgtgtcgtctctgtcgccgtccccgtctcctgccaggcgcggagccctgcgagccgcgggtgggccccaggcgcgcagacatgggctgctccgccaaagcgcgctgggctgccggggcgctgggcgtcgcggggctactgtgcgctgtgctgggcgctgtcatgatcgtgatggtgccgtcgctcatcaagcagcaggtccttaagaacgtgcgcatcgaccccagtagcctgtccttcaacatgtggaaggagatccctatccccttctatctctccgtctacttctttgacgtcatgaaccccagcgagatcctgaagggcgagaagccgcaggtgcgggagcgcgggccctacgtgtacagggagttcaggcacaaaagcaacatcaccttcaacaacaacgacaccgtgtccttcctcgagtaccgcaccttccagttccagccctccaagtcccacggctcggagagcgactacatcgtcatgcccaacatcctggtcttgggtgcggcggtgatgatggagaataagcccatgaccctgaagctcatcatgaccttggcattcaccaccctcggcgaacgtgccttcatgaaccgcactgtgggtgagatcatgtggggctacaaggacccccttgtgaatctcatcaacaagtactttccaggcatgttccccttcaaggacaagttcggattatttgctgagctcaacaactccgactctgggctcttcacggtgttcacgggggtccagaacatcagcaggatccacctcgtggacaagtggaacgggctgagcaaggttgacttctggcattccgatcagtgcaacatgatcaatggaacttctgggcaaatgtggccgcccttcatgactcctgagtcctcgctggagttctacagcccggaggcctgccgatccatgaagctaatgtacaaggagtcaggggtgtttgaaggcatccccacctatcgcttcgtggctcccaaaaccctgtttgccaacgggtccatctacccacccaacgaaggcttctgcccgtgcctggagtctggaattcagaacgtcagcacctgcaggttcagtgcccccttgtttctctcccatcctcacttcctcaacgctgacccggttctggcagaagcggtgactggcctgcaccctaaccaggaggcacactccttgttcctggacatccacccggtcacgggaatccccatgaactgctctgtgaaactgcagctgagcctctacatgaaatctgtcgcaggcattggacaaactgggaagattgagcctgtggtcctgccgctgctctggtttgcagagagcggggccatggagggggagactcttcacacattctacactcagctggtgttgatgcccaaggtgatgcactatgcccagtacgtcctcctggcgctgggctgcgtcctgctgctggtccctgtcatctgccaaatccggagccaagagaaatgctatttattttggagtagtagtaaaaagggctcaaaggataaggaggccattcaggcctattctgaatccctgatgacatcagctcccaagggctctgtgctgcaggaagcaaaactgtagggtcctgaggacaccgtgagccagccaggcctggccgctgggcctgaccggccccccagcccctacaccccgcttctcccggactctcccagcggacagccccccagccccacagcctgagcctcccagctgccatgtgcctgttgcacacctgcacacacgccctggcacacatacacacatgcgtgcaggcttgtgcagacactcagggatggagctgctgctgaagggacttgtagggagaggctcgtcaacaagcactgttctggaaccttctctccacgtggcccacaggcctgaccacaggggctgtgggtcctgcgtccccttcctcgggtgagcctggcctgtcccgttcagccgttgggcccaggcttcctcccctccaaggtgaaacactgcagtcccggtgtggtggctccccatgcaggacgggccaggctgggagtgccgccttcctgtgccaaattcagtggggactcagtgcccaggccctggccacgagctttggccttggtctacctgccaggccaggcaaagcgcctttacacaggcctcggaaaacaatggagtgagcacaagatgccctgtgcagctgcccgagggtctccgcccaccccggccggactttgatccccccgaagtcttcacaggcactgcatcgggttgtctggcgcccttttcctccagcctaaactgacatcatcctatggactgagccggccactctctggccgaagtggccgcaggctgtgcccccgagctgcccccaccccctcacagggtccctcagattataggtgcccaggctgaggtgaagaggcctgggggccctgccttccgggcgctcctggaccctggggcaaacctgtgacccttttctactggaatagaaatgagttttatcatctttgaaaaataattcactcttgaagtaataaacgtttaaaaaaatgggaaaaaaaaaaaaaaaaaa

SEQ ID NO: 28 - DNA, 2184 bp, Homo sapiens
  source 1..2184: mol_type = genomic DNA
gtgcgcgaacggctccggcccgcacgggtcgccagaggcgactgtgtgacactcggagtttgctggggtctccgtgggcgggaggactttccagcgcaatggcgactccctaagccccgcagcttctgcgcccgggaaagatatccaagagatgcaaagctctactgggcccaggctgccaccccagaggcccccttccgtcccggggccggggctaggccaaggcgggcaccaggactgcccagcctcccggcccttcgcactggtaaccggttccggggcggatgctttttgcatctgacccggcgcgcccggtgacgccttcgcgtccagacggaagtgcgggcggaggatccccagccgggtcccaagcctgtgcctgagcctgagcctgagcctgagcccgagccgggagccggtcgcgggggctccgggctgtgggaccgctgggcccccagcgatggcgaccctgtggggaggccttcttcggcttggctccttgctcagcctgtcgtgcctggcgctttccgtgctgctgctggcgcagctgtcagacgccgccaagaatttcgaggatgtcagatgtaaatgtatctgccctccctataaagaaaattctgggcatatttataataagaacatatctcagaaagattgtgattgccttcatgttgtggagcccatgcctgtgcgggggcctgatgtagaagcatactgtctacgctgtgaatgcaaatatgaagaaagaagctctgtcacaatcaaggttaccattataatttatctctccattttgggccttctacttctgtacatggtatatcttactctggttgagcccatactgaagaggcgcctctttggacatgcacagttgatacagagtgatgatgatattggggatcaccagccttttgcaaatgcacacgatgtgctagcccgctcccgcagtcgagccaacgtgctgaacaaggtagaatatgcacagcagcgctggaagcttcaagtccaagagcagcgaaagtctgtctttgaccggcatgttgtcctcagctaattgggaattgaattcaaggtgactagaaagaaacaggcagacaactggaaagaactgactgggttttgctgggtttcattttaataccttgttgatttcaccaactgttgctggaagattcaaaactggaagcaaaaacttgcttgatttttttttcttgttaacgtaataatagagacatttttaaaagcacacagctcaaagtcagccaataagtcttttcctatttgtgacttttactaataaaaataaatctgcctgtaaattatcttgaagtcctttacctggaacaagcactctctttttcaccacatagttttaacttgactttcaagataattttcagggtttttgttgttgttgttttttgtttgtttgttttggtgggagaggggagggatgcctgggaagtggttaacaacttttttcaagtcactttactaaacaaacttttgtaaatagaccttaccttctattttcgagtttcatttatattttgcagtgtagccagcctcatcaaagagctgacttactcatttgacttttgcactgactgtattatctgggtatctgctgtgtctgcacttcatggtaaacgggatctaaaatgcctggtggcttttcacaaaaagcagattttcttcatgtactgtgatgtctgatgcaatgcatcctagaacaaactggccatttgctagtttactctaaagactaaacatagtcttggtgtgtgtggtcttactcatcttctagtacctttaaggacaaatcctaaggacttggacacttgcaataaagaaattttattttaaacccaagcctccctggattgataatatatacacatttgtcagcatttccggtcgtggtgagaggcagctgtttgagctccaatgtgtgcagctttgaactagggctggggttgtgggtgcctcttctgaaaggtctaaccattattggataactggcttttttcttcctatgtcctctttggaatgtaacaataaaaataatttttgaaacatccatcagtgtatctatctatgtctcctagttttttcctcctccctcttttgctgtataatgagattgaagatataaagacattttgtaccctgtaaaaaaaa

SEQ ID NO: 29 - DNA, 5135 bp, Homo sapiens
  source 1..5135: mol_type = genomic DNA
aactcaaggcctgcttgatacgtccgccattttgggcgcttcgctgatggtgtcggtgagcgcgtttcccgcctgagcgcaactagcggcgggtcgtgggcacctccaggagagcttgtttcatatccatatcccactgtattcctgctaatctgctaatgcagtaaattggaggaaaactgttaccaggataacctgtaatgggcaaggagccacaaagaagaaaacatttcttttaatttttaaacttggtttgaaagaccagcatgttttggaaatttgatcttcactcatcatcccacatagacacacttctagaaagagaagatgtaacactgaaggagttaatggatgaggaagatgttttacaggaatgtaaagctcagaaccgcaaacttatagagtttctgttaaaagcagaatgtctcgaagatttagtctcattcattatagaagaaccacctcaagacatggatgaaaagatcagatacaagtatccaaatatatcttgtgagttgctcacttctgatgtctcccagatgaatgatagactgggagaagatgaatccttgctaatgaaattatatagcttcctcctaaacgattcccctttgaatccactacttgccagtttcttcagcaaggtgctaagtattcttatcagcagaaaaccagaacagattgtggatttcttaaagaagaagcatgattttgtagaccttattataaagcacataggaacttctgctatcatggatttgttgctcaggctcctgacgtgtatcgaacctccacagcccaggcaagatgtgctgaattggttaaatgaggagaaaattatccagaggcttgtggaaatagttcatccatcgcaagaagaagatcgacattcaaatgcatcacaatcactttgtgaaattgttcgcctgagcagagaccagatgttacaaattcagaacagtacagagcccgaccccctgcttgccactctagaaaagcaagaaattatagagcagcttctatcaaatattttccacaaggagaaaaatgagtcagccatagtcagtgcaatccagatattgctgactttacttgagacacgacgaccaacatttgaaggccatatagagatctgcccaccaggcatgagccattcagcttgttcagtaaacaagagtgttctagaagccatcagaggaagacttggatcttttcatgaactcctgctggagccacccaagaaaagtgtgatgaagaccacatggggtgtgctggatcctcctgtggggaatacccggttgaatgtcattaggttgatatccagcctgcttcaaaccaataccagcagtataaatggggaccttatggagctgaatagcattggagtcatattgaacatgttcttcaagtatacatggaataactttttgcatacacaagtggaaatttgtattgcactgattcttgcaagtccttttgaaaacacagaaaatgccacaattaccgatcaagactccactggtgataatttgttattaaaacatcttttccaaaaatgtcaattaatagaacgaatacttgaagcctgggaaatgaatgagaagaaacaggctgagggaggaagacggcatggttacatgggacacctaacgaggatagctaactgtatcgtgcacagcactgacaagggccccaacagtgcattagtgcagcagcttatcaaagatcttcccgacgaagtcagggaacgatgggagacgttctgcacaagctccttaggagaaactaacaagaggaacacggtagatctagttacaacctgccatattcattcatccagtgatgatgaaattgactttaaagaaacgggtttctcacaggattcttctttgcagcaagccttttctgattatcagatgcaacaaatgacgtccaattttattgaccagtttggcttcaacgatgagaagtttgcagatcaagatgacattggcaatgtttcttttgatcgagtatcagacatcaactttactctcaatacaaatgaaagtggaaatattgccttgtttgaagcatgttgtaaggaaagaatacaacagtttgatgatggtggctctgatgaggaagatatatgggaggaaaagcacatcgcattcacaccagaatcccaaagacgatccagctcggggagtacagacagtgaggaaagtacagactctgaagaagaagatggagcaaagcaagacttgtttgaacccagcagtgccaacacggaggataaaatggaggtggacctgagtgaaccacccaactggtcagctaactttgatgtcccaatggaaacaacccacggtgctccattggattctgtgggatctgatgtctggagcacagaggagccgatgccaactaaagagacgggctgggcttctttttcagagttcacgtcttccctgagcacaaaagattctttaaggagtaattctccagtggaaatggaaaccagcactgaacccatggaccctctgactcccagtgcggctgccctggcagtgcagccagaagcggcaggcagtgtggccatggaagccagctctgacggagaggaggatgcagaaagtacagacaaggtaactgagacagtgatgaatggcggcatgaaggaaacgctcagcctcactgtagatgccaagacagagactgcggtcttcaaaagtgaggaagggaaactgtctacctctcaagatgctgcttgtaaagacgcagaggagtgtcccgagactgcagaggcgaagtgcgcggcgcccaggcctcccagcagcagtcccgagcagagtgcctccgatgcctgtctgttgctccttaggactggccaaccaagcgcaccaggtgacacttcagtgaatggccctgtatgacgggtgacgtctgctgctgctgactgaggactgcagaccgccaccactcaggggctctggaggggtcagctggagcccaccaagctgtcactgctgcactcactctgcaagggatcaggaccagcaacctttatattctagattctaagacattgtacagagaaattcagaagtgtaaaaatattgcacattgacaaataccaagaatttttgcgtatgtttatattgtattgttctaaataatgggtagcctgtgaaataagatcttgccacccatgtaataatagtagtaatactatagttaaaatggctgtaagaatagttttataaaagtgaatacacagatctattgtatttgaaacataactttgacaattattagtgtgaccaaagtattaggcggttttcatacatttttcaccttgtacaaaattatgaattcatttttcctccaggccgacaaggagttgtagaatgaaaatgccctctaagtgttattttggttgttctaacttacaaaagtgattttgaataagaaatatttggtgttctttttataaccagtttttgattggtaattgttttctgtattgtttaaaacggatcaaaaatgtaagtctattggtagagattaagtaaagtatttattgctacatcatagttgataaattgatgttatcgtaaagccatatgttctgttcaagtcttgtttgcttgaaatgattattcctacaagtgaaacactagactatttggagtgtatatggcttgtgttttgggattttttttttttttttttggcttttgtttttgtttgtttttttgtttcatttggtagttcatctgccttttaacccattcaccaaaatttaccttgttaacaagcatcaccaatgaacatttcagagcaatctgcatatttaacagacctaaaataaatcctattaggcaagtcagttgaaaatgctcgtgctgctaatggaattagagtgcgttcattttacaggctagtattttaaaagtagaaatcaaaatctggcaccgaagcatgctaattgtttactgtaccttgtgaggttttcactcataaatttaaaccagtgtatttttttagaactggtttgtgtatatatatagtgattatggatactaattcaatgtaatttataattttctatgtcaatacaaaaatacatcacagccttctcaaacagctcaagcaatatattgtatattgccatatcgtctggtgaaagggttaaattacttcacctcttgcacttttagatgcaaatcagtttttcatttctgtaatagaaaattattcacgtatttttacatcatttgtttttcctgaccagtatttaaaaccaaaaggatattctgaaaaatggccaacaatttttttagaagtagcatcccaagcagcgtgcctaaacattacattgcatatggaaataaaagaatcaaacgtctaatgccttattatttctgatttcctttttcattttaagtggtgtggagattccagcactcccaggacagtggagtcagcagtaagccctgggacaggtggcaagggtgggtcccttgacctttgcacgcctcctcaggaaccccctttcccgggtgagcccctctctgaagagactgtccttgggcctcctctggaagcagcacccccagaggacagggctcctcctgcttgcctcagggctgcctgacttgaatggcgttggacctcggggattactggtagataatatgctctggtctcgcctggtggtgagttttgccagccatggccagggtttggctccactggtggcacacgtggcctccgtggtatggacctggtggcttctccatcccactgtggcctctgtggtatggacctggtggcttctccatcctacccaaggtaacagtgtcttgcttcatcccactgactgctgggagagagcctctgggacttttctttggggcatcattttgttttgtctttcgtagcagggaaaggatatgacaatggggaggacagttcttttggaggttggaggggccaagccaaggacaggagcaagtgtgccctcattttgtttctacttttaatttctgtgtgttggccatactgaattatgagactaacagatgtctacaatacaatacctgtattcaaaataacaaaaataaagcctgattctttgtttctagaaa

SEQ ID NO: 30 - DNA, 76 bp, Homo sapiens
  source 1..76: mol_type = genomic DNA
tgagggaggaagacggcatggttacatgggacacctaacgaggatagctaactgtatcgtgcacagcactgacaag

SEQ ID NO: 31 - DNA, 66 bp, Homo sapiens
  source 1..66: mol_type = genomic DNA
gacagatttctaccactccaaacgccggctgatcttctccaagaggaagccctaatccgcccacag